(19) United States
(12) Patent Application Publication
(10) Pub. No.: US 2019/0365664 A1
(43) Pub. Date: Dec. 5, 2019

(54) CARYOPHYLLENE FORMULATIONS, APPARATUSES, AND METHODS

(71) Applicant: Biotech Institute, LLC, Westlake Village, CA (US)

(72) Inventors: Gary Scott HILLER, Westlake Village, CA (US); Mark Anthony LEWIS, Westlake Village, CA (US)

(21) Appl. No.: 16/461,870

(22) PCT Filed: Nov. 20, 2017
(86) PCT No.: PCT/US2017/062623

§ 371 Date: May 17, 2019

Related U.S. Application Data
(60) Provisional application No. 62/424,327, filed on Nov. 18, 2016.

(51) Int. Cl. A61K 31/015; A61K 9/00; A61M 11/04

(52) U.S. Cl. CPC A61K 31/015; A61M 11/041; A61K 9/0078

(57) ABSTRACT

Apparatuses, methods, and compositions for administering vaporized caryophyllene compositions to a human subject in need thereof. The caryophyllene compositions can be administered via a handheld electronic device that vaporizes pure, essentially pure, or substantially pure caryophyllene (beta caryophyllene and/or alpha caryophyllene) in small doses for inhalation by a user. Some compositions do not include or contain additional constituents, carriers, or additives, and demonstrate beneficial physiological effects that are not seen when alpha caryophyllene or beta caryophyllene is administered orally or by injection.

### CROSS-REFERENCE TO RELATED APPLICATIONS

The present application claims the benefit of priority to U.S. Provisional Application No. 62/424,327, filed on Nov. 18, 2016, the contents of which is hereby incorporated by reference in their entirety.

### FIELD OF THE INVENTION

The present invention relates to caryophyllene compositions, apparatuses comprising the caryophyllene compositions and methods of using caryophyllene compositions.

### BACKGROUND

Beta-caryophyllene is an ingestible food additive that is generally recognized as safe for human consumption by the Food and Drug Administration. Recent studies of beta-caryophyllene have started to uncover potential medical applications for high doses of oral or parenteral formulations of the drug. Currently available forms of beta-caryophyllene require relatively high doses and also result in delayed, and less predictable pharmacokinetics for the drug. Current uses for beta-caryophyllene are thus likely to result in high costs, and low patient adherence to treatment schedules.

The present inventions address many of the shortcomings of the present technology, and provide patients with new formulations, devices, and methods of treatment for unlocking caryophyllene's potential.

### SUMMARY OF THE INVENTION

The present disclosure provides, among other things, vaporizable caryophyllene compositions for use in a human subject in need thereof. Some embodiments provide low-dose vaporizable caryophyllene compositions for use in a human subject in need thereof.

In some embodiments, the vaporizable caryophyllene compositions comprise beta caryophyllene. In further embodiments, the vaporizable beta caryophyllene compositions comprise pure, essentially pure, or substantially pure beta caryophyllene. In additional embodiments, the vaporizable beta caryophyllene compositions comprise beta caryophyllene and alpha caryophyllene. Certain further embodiments provide low-dose beta caryophyllene compositions for use in a human subject in need thereof.

In some embodiments, the vaporizable caryophyllene compositions comprise at least 60%, at least 70%, at least 80%, at least 90%, at least 95%, or at least 99% beta caryophyllene. In some embodiments, the vaporizable caryophyllene composition consists of or consists essentially of beta caryophyllene.

In some embodiments, the vaporizable caryophyllene compositions comprise alpha caryophyllene. In further embodiments, the vaporizable alpha caryophyllene compositions comprise pure, essentially pure, or substantially pure alpha caryophyllene. In additional embodiments, the vaporizable alpha caryophyllene compositions comprise beta caryophyllene and alpha caryophyllene. Certain further embodiments provide low-dose alpha caryophyllene compositions for use in a human subject in need thereof.

In some embodiments, the caryophyllene composition comprises at least 60%, at least 70%, at least 80%, at least 90%, at least 95%, or at least 99% alpha caryophyllene. In some embodiments, the vaporizable caryophyllene composition consists of or consists essentially of alpha caryophyllene.

In some embodiments, the vaporizable caryophyllene compositions comprise a mixture of alpha caryophyllene and beta caryophyllene. In further embodiments, the vaporizable mixtures comprise pure, essentially pure, or substantially pure mixtures of alpha caryophyllene and beta caryophyllene (in various ratios provided in the present disclosure). In some embodiments, the vaporizable caryophyllene composition consists of or consists essentially of a mixture of alpha caryophyllene and beta caryophyllene.

In some embodiments, the vaporizable caryophyllene compositions do not include or contain additional constituents, carriers, or additives. In certain embodiments, the vaporizable caryophyllene compositions that do not include or contain additional constituents, carriers, or additives show beneficial and/or desirable physiological effects that are not seen when larger doses of alpha caryophyllene and/or beta caryophyllene are administered orally or by injection.

The present disclosure provides methods for administering the caryophyllene compositions of the present disclosure to a human subject in need thereof using a device that vaporizes the composition. In certain embodiments, the device is a handheld electronic device.

The present disclosure provides an apparatus for administering atomized or vaporized caryophyllene to a human subject in need thereof. In some embodiments, the atomized or vaporized caryophyllene is low-dose caryophyllene.

In some embodiments, the apparatus comprises:

(a) a caryophyllene reservoir configured to hold a liquid caryophyllene composition;

(b) a caryophyllene atomizer in fluid communication with the caryophyllene reservoir;

(c) an actuator coupled to the caryophyllene atomizer and configured such that, when actuated, the actuator initiates atomization of liquid caryophyllene composition at the caryophyllene atomizer;

(d) a caryophyllene vapor chamber in fluid communication with the caryophyllene atomizer and configured to receive atomized caryophyllene composition; and

(e) a mouthpiece in fluid communication with the caryophyllene vapor chamber and configured to provide atomized composition of caryophyllene from the caryophyllene vapor chamber to a user.

The present disclosure provides methods of treatment comprising using the apparatus to administer a low dose atomized or vaporized caryophyllene compositions to a patient in need thereof.

In some embodiments, the methods of treatment provide a dosage of atomized or vaporized caryophyllene composition of less than 10 mg, less than 9 mg, less than 8 mg, less than 7 mg, less than 6 mg, less than 5 mg, less than 4 mg, less than 3 mg, less than 2.5 mg, less than 2 mg, less than 1.5 mg, less than 1 mg, or less than 0.5 mg of vaporized caryophyllene composition.

In some embodiments, the methods of treatment provide a dosage of atomized or vaporized caryophyllene composition of less than 10 mg, less than 9 mg, less than 8 mg, less than 7 mg, less than 6 mg, less than 5 mg, less than 4 mg, less than 3 mg, less than 2.5 mg, less than 2 mg, less than 1.5 mg, less than 1 mg, or less than 0.5 mg of vaporized beta caryophyllene.

In some embodiments, the methods of treatment provide a dosage of atomized or vaporized caryophyllene composition of less than 10 mg, less than 9 mg, less than 8 mg, less than 7 mg, less than 6 mg, less than 5 mg, less than 4 mg, less than 3 mg, less than 2.5 mg, less than 2 mg, less than 1.5 mg, less than 1 mg, or less than 0.5 mg of vaporized alpha caryophyllene.

The present compositions, apparatuses and methods, and advantages thereof, are further illustrated by the following non-limiting detailed description and Examples.

### BRIEF DESCRIPTION OF THE DRAWINGS

FIG. 1 illustrates the Product Feedback Survey scale from Example 3.

FIG. 2 is a graphical representation of the baseline subtracted volunteer results from the Product Feedback Survey from Example 3.

### DEFINITIONS

All definitions, as defined and used herein, should be understood to control over dictionary definitions, definitions in documents incorporated by reference, and/or ordinary meanings of the defined terms. It should also be appreciated that terminology explicitly employed herein that also may appear in any disclosure incorporated by reference should be accorded a meaning most consistent with the particular concepts disclosed herein.

The herein described subject matter sometimes illustrates different components contained within, or connected with, different other components, such as with the BCP device. It is to be understood that such architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedia components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components of a BCP device.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of.” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, in particular embodiments, the terms “about” or “approximately” when preceding a numerical value indicates the value plus or minus a range of 10%.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure. Where a list of values is provided, it is understood that ranges between any two values in the list are also contemplated as additional embodiments encompassed within the scope of the disclosure, and it is understood that each intervening value to the tenth of the unit of the lower limit unless the context clearly dictates otherwise, between the upper and lower limit of said range and any other listed or intervening value in said range is encompassed within the disclosure; that the upper and lower limits of said sub-ranges can independently be included in the sub-ranges is also encompassed within the disclosure, subject to any specifically excluded limit.

### DETAILED DESCRIPTION

The present inventions, are based in part on the inventor's discovery that low doses of caryophyllene administered via inhalation produce beneficial medical and recreational effects not exhibited by traditional oral or parenteral administrations of caryophyllene. The inventors discovered for example, that vaporizable compositions of caryophyllene (BCP, ACP, and/or combinations thereof) could produce psychoactive effects with potential medical and recreational applications.

Following below are more descriptions of various concepts related to, and embodiments of, methods, compositions, and apparatuses for caryophyllene. It should be appreciated that various concepts disclosed can be implemented in any of numerous ways, as the disclosed concepts are not limited to any particular manner of implementation. Examples of specific implementations and applications are provided primarily for illustrative purposes.

### Caryophyllene Compounds

Beta-caryophyllene (BCP; CAS 87-44-5; 4,11,11-Trimethyl-8-methylenebicyclo[7.2.0]undec-4-ene; β-Caryophyllene; trans-(1R,9S)-8-Methylene-4,11,11-trimethylbicyclo[7.2.0]undec-4-ene) is a CB2-receptor agonist (Gertsch et al. 2008, Anavi-Goffer et al., 2012, herein expressly incorporated by reference). Orally administered BCP is absorbed by the digestive tract and becomes systemically available. When administered orally, BCP has anti-inflammatory and gastric cytoprotective properties, and may even have anti-malarial activity. For example, WIPO Patent Application Publication WO/2013/184036, incorporated herein by reference for all purposes, discloses that large oral doses of BCP can be used as a liver treatment. Other studies have indicated that BCP does not have psychoactive effects (Marijuana Has Anti-Inflammatory That Won't Get You High, Richard A. Lovett, National Geographic News, Jun. 24, 2008).

BCP is a natural bicyclic sesquiterpene that is a constituent of many essential oils, including cloves, Cannabis sativa, rosemary, and hops. In general, naturally occurring BCP is found as a mixture with isocaryophyllene (the cis olefin isomer) and α-humulene (a ring-opened isomer). BCP is often the predominant sesquiterpenoid in cannabis. It is less volatile than the monoterpenoids, and, thus, is found in higher concentrations in material that has been processed by heat to aid in decarboxylation. It is a selective full agonist at the CB2 receptor, which makes it the only phytocannabinoid found outside the cannabis genus. BCP has the following structural formula:

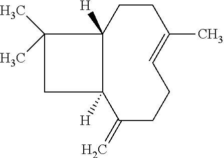

Alpha-caryophyllene (ACP; 2,6,6,9-Tetramethyl-1,4-8-cycloundecatriene; also known as humulene, α-humulene or α-caryophyllene), is a naturally occurring monocyclic sesquiterpene, having an 11-membered ring and consisting of 3 isoprene units containing three non-conjugated olefins, two of which are tri-substituted and one of which is di-substituted. ACP is an isomer of BCP. ACP has the following structural formula:

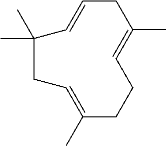

As detailed in the present disclosure, the Inventors have found beneficial and unexpected effects from administration of inhaled caryophyllene compositions.

### Compositions

In some embodiments, the present disclosure teaches vaporizable compositions. In some embodiments, the vaporizable compositions of the present disclosure comprise BCP. In other embodiments, the vaporizable compositions of the present disclosure comprise ACP. In yet other embodiments, the vaporizable compositions of the present disclosure comprise both BCP and ACP.

In some embodiments, the vaporizable compositions are designed exist outside of cannabis regulatory frameworks. Thus, in some embodiments, the vaporizable compositions of the present disclosures are free, or substantially free of THC and/or CBD. In other embodiments, the vaporizable compositions are free of any cannabinoid. Thus in some embodiments, the vaporizable compositions of the present disclosure do not comprise tetrahydrocannabivarin (THCv), cannabidivarin (CBDv), cannabichromene (CBC), cannabigerol (CBG), cannabigevarin (CBGv).

In some embodiments, the vaporizable composition is free from, or substantially free from one or more other terpenes other than BCP and ACP.

In some embodiments, the vaporizable compositions of the present disclosure are formulated for vaporizable delivery (i.e. via inhalation of vaporized particles produced via a heating element or atomizer). Thus, in some embodiments, the vaporizable compositions comprise one or more diluents and/or carriers (e.g., e-liquid).

E-liquid, e-juice, or simply “juice”, refers to a liquid solution that produces a mist or vapor when heated by an atomizer. In some embodiments, the main ingredients of e-liquids are usually a mix of propylene glycol (PG), vegetable glycerin (VG), and/or polyethylene glycol 400 (PEG400), sometimes with differing levels of alcohol mixed with concentrated or extracted flavorings; and a variable concentration of caryophyllene. Thus, in some embodiments, the vaporizable compositions of the present disclosure comprise one or more of propylene glycol, glycerine, vegetable glycerine, and/or water.

In some embodiments, the carrier comprises one or more medium chain length triglycerides (MCTs). MCTs are triglycerides whose fatty acids have an aliphatic tail of 6-12 carbon atoms. In certain embodiments, the MCT is one or more of caproic acid, caprylic acid, capric acid, lauric acid and mixtures thereof. Suitable sources of MCTs are known to those skilled in the art and include, for example, coconut oil and palm kernel oil.

In some embodiments, the carrier comprises one or more polyesterdiols. The polyesterdiol may be a linear two to ten units polymer (also referred to as (ester)₂₋₁₀ glycol), that is derived from natural or non-natural sources such as vegetables, fruits, bacteria, yeast, algae, or manufactured by chemical processes.

For example, in some embodiments, the polyesterdiol is 1) a polypropylene glycol such as: dipropylene glycol; tripropylene glycol, including tetra-, penta-, hexa-, hepta-, octa-, nona- and decapropylene glycol and other derivatives thereof; 2) a polybutylene glycol such as: dibutylene glycol, tributylene glycol, including tetra-, penta-, hexa-, hepta-, octa-, nona- and decabutylene glycol, and other derivatives thereof; 3) also including 2-10 unit polymers of rare organic ester types such as pentylene, octylene, terpentylene, nonylene, linalylene, isoamylene, isobutylene, geranylene, bornylene, benzylene and allylene, caprylylene, such as, for example, polyisobutylene glycol such as diisobutylene glycol; and 4) triethylene glycol, including tetra-, penta-, hexa-, hepta-, octa-, nona- and decaethylene glycols and other derivatives thereof such as acid or sugar conjugates, and esters or ether or alcohol derivatives.

In some embodiments, the carrier is a linear polyesterdiol selected from the group consisting of: (ethylene)₃₋₁₀ glycol; (propylene)₂₋₁₀ glycol; (butylene)₂₋₁₀ glycol; (pentylene)₂₋₁₀ glycol; (octylene)₂₋₁₀ glycol; (terpentylene)₂₋₁₀ glycol; (nonylene)₂₋₁₀ glycol; (linalylene)₂₋₁₀ glycol; (isoamylene)₂₋₁₀ glycol; (isobutylene)₂₋₁₀ glycol; (geranylene)₂₋₁₀ glycol; (bornylene)₂₋₁₀ glycol; (benzylene)₂₋₁₀ glycol; (allylene)₂₋₁₀ glycol; and (caprylylene)₂₋₁₀ glycol; acid or sugar conjugates thereof, and ester or ether or alcohol derivatives thereof.

In some embodiments, the carrier is selected from the group consisting of: triethylene glycol; tetraethyleneglycol, pentaethylenglycol, hexaethyleneglycol, heptaethyleneglycol, octaethyleneglycol, nonaethylenglycol; decaethylene glycol; dipropylene glycol; tripropylene glycol; tetrapropylene glycol, pentapropylene glycol, hexapropylene glycol, heptapropylene glycol, octapropylene glycol, nonapropylene glycol; decapropylene glycol; dibutylene glycol, tributylene glycol; tetrabutylene glycol, pentabutylene glycol, hexabutylene glycol, heptabutylene glycol, octabutylene glycol, nonabutylene glycol, decabutylene glycol and diisobutylene glycol.

In some embodiments, the carrier is selected from the group consisting of borneol, camphor, 1,8-Cineole, citral, geraniol, indomethacin, limonene, linalool, linalyl acetate, β-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone

If desired, one or more flavorants may be added to the composition. Non-limiting examples include wintergreen, propolis, eucalyptus, tobacco, Turkish tobacco, menthol, peppermint, spearmint, green tea, cream, natural sweetener, banana, strawberry, blueberry, mango, raspberry, peach, pineapple, apple, orange, papaya, watermelon, grape, cherry, coffee, chocolate, vanilla, cola, piña colada, coconut, almond, cinnamon, white chocolate, rum, butterscotch, and caramel or the like.

In some embodiments, additional components are optionally added to the caryophyllene composition to improve the taste and/or physical properties of the composition (such as stability, viscosity, appearance of smoke as it is exhaled, etc.). Such additional components include, but are not limited to, sweeteners, natural flavorants, artificial flavorants, colorants, antioxidants, preservatives, chelating agents, viscomodulators, tonicifiers, odorants, opacifiers, suspending agents, binders, thickeners, carriers and mixtures thereof, including, but not limited to, xanthum gum, carboxymethylcellulose, carboxyethylcellulose, hydroxypropylcellulose, methylcellulose, microcrystalline cellulose, starches, dextrins, maltodextrins, other polyols (including sugar alcohols, such as sorbitol, lactitol or mannitol), carbohydrates (e.g., lactose), propylene glycol alginate, gellan gum, guar, pectin, tragacanth gum, gum acacia, locust bean gum, gum arabic, mannitol, sucralose, silicon dioxide, stearic acid, hydroxypropyl methylcellulose, mono-, di- and triglycerides (acyl glycerols), ether and sugar acetates or other acid esters such as dimethyl acetate, ethyl acetate, isopropyl acetate, ethylhexyl acetate, butyl acetate, triethyl citrate, dimethyl butyrate and the like.

In some embodiments, the vaporizable compositions of the present disclosure comprise less than 1% wt. wt, less than 2% wt., less than 3% wt., less than 4% wt., less than 5% wt., less than 6% wt., less than 7% wt., less than 8% wt., less than 9% wt., less than 10% wt., less than 11% wt, less than 12% wt., less than 13% wt., less than 14% wt., less than 15% wt., less than 16% wt., less than 17% wt, less than 18% wt, less than 19% wt., less than 20% wt., less than 21% wt., less than 22% wt, less than 23% wt., less than 24% wt., less than 25% wt., less than 26% wt., less than 27% wt., less than 28% wt., less than 29% wt., less than 30% wt., less than 31% wt., less than 32% wt., less than 33% wt., less than 34% wt., less than 35% wt., less than 36% wt., less than 37% wt., less than 38% wt., less than 39% wt., less than 40% wt., less than 41% wt., less than 42% wt., less than 43% wt., less than 44% wt., less than 45% wt., less than 46% wt., less than 47% wt., less than 48% wt., less than 49% wt., less than 50% wt., less than 51% wt, less than 52% wt., less than 53% wt., less than 54% wt., less than 55% wt., less than 56% wt., less than 57% wt., less than 58% wt., less than 59% wt., less than 60% wt., less than 61% wt., less than 62% wt., less than 63% wt., less than 64% wt., less than 65% wt., less than 66% wt., less than 67% wt., less than 68% wt., less than 69% wt., less than 70% wt., less than 71% wt., less than 72% wt., less than 73% wt., less than 74% wt., less than 75% wt., less than 76% wt., less than 77% wt., less than 78% wt., less than 79% wt., less than 80% wt., less than 81% wt., less than 82% wt., less than 83% wt., less than 84% wt., less than 85% wt., less than 86% wt., less than 87% wt., less than 88% wt., less than 89% wt., less than 90%/c wt., less than 91% wt., less than 92% wt., less than 93% wt., less than 94% wt., less than 95% wt., less than 96% wt., less than 97% wt., less than 98% wt., less than 99% wt., or less than 100% wt. beta caryophyllene and/or alpha caryophyllene.

In some embodiments, the present disclosure teaches that lower concentrations of beta caryophyllene are better tolerated by patients. In some embodiments, the present application teaches that (counterintuitively), lower concentration vaporizable compositions of the present disclosure are superior to higher concentrations. Thus in some embodiments, the vaporizable compositions of the present disclosure comprise between 1%-75% wt. beta caryophyllene. In some embodiments, the vaporizable compositions of the present disclosure comprise between 1%-50% wt. beta caryophyllene. In some embodiments, the vaporizable compositions of the present disclosure comprise between 1%-25% wt. beta caryophyllene. In some embodiments, the vaporizable compositions of the present disclosure comprise between 1%-10% wt. beta caryophyllene.

In some embodiments, the present disclosure teaches that lower concentrations of alpha caryophyllene are better tolerated by patients. In some embodiments, the present application teaches that (counterintuitively), lower concentration vaporizable compositions of the present disclosure are superior to higher concentrations. Thus in some embodiments, the vaporizable compositions of the present disclosure comprise between 1%-75% wt. alpha caryophyllene. In some embodiments, the vaporizable compositions of the present disclosure comprise between 1%-50% wt. alpha caryophyllene. In some embodiments, the vaporizable compositions of the present disclosure comprise between 1%-25% wt. alpha caryophyllene. In some embodiments, the vaporizable compositions of the present disclosure comprise between 1%-10% wt. alpha caryophyllene.

### Caryophyllene Delivery Devices

The present disclosure provides methods for administering the vaporizable compositions of the present disclosure to a human subject in need thereof using a device that vaporizes the composition. In certain embodiments, the device is a handheld electronic device.

The present disclosure provides an apparatus for administering atomized or vaporized caryophyllene to a human subject in need thereof. In some embodiments, the atomized or vaporized caryophyllene is low-dose caryophyllene.

In some embodiments, the caryophyllene delivery device comprises: (a) a caryophyllene reservoir configured to hold a liquid caryophyllene composition; (b) a caryophyllene atomizer in fluid communication with the caryophyllene reservoir; (c) an actuator coupled to the caryophyllene atomizer and configured such that, when actuated, the actuator initiates atomization of liquid caryophyllene composition at the caryophyllene atomizer; (d) a caryophyllene vapor chamber in fluid communication with the caryophyllene atomizer and configured to receive atomized caryophyllene composition; and (e) a mouthpiece in fluid communication with the caryophyllene vapor chamber and configured to provide atomized composition of caryophyllene from the caryophyllene vapor chamber to a user.

In some embodiments, components of a BCP device include a mouthpiece, a BCP reservoir (which can be a tank or cartridge), a heating element or other atomizer (e.g., piezoelectric atomizer, inkjet-type atomizer, etc.), and an actuator (such as a switch or pressure sensor) to activate the heating element/atomizer. Depending on the embodiment, the BCP reservoir can be configured to store between about 0.5 mg and 500 mg of a BCP composition/BCP liquid, for example, between about 1 mg and about 250 mg, about 2 mg and 150 mg, about 3 mg and about 100 mg, about 4 mg and about 75 mg, about 5 mg and about 50 mg, or between about 10 mg and about 30 mg.

Some implementations of the BCP device can include a microprocessor and a battery or other power source. Some implementations include and/or define a BCP vapor chamber or BCP atomization chamber. In some embodiments, an atomizer can include a small heater (e.g., heat coil or other heat element), that vaporizes a liquid BCP composition stored in the BCP reservoir. Some embodiments include a one or more wicks or wicking material configured to draw liquid BCP composition onto or into the heater or atomizer. In some embodiments, when a user activates an actuator (i.e., activates a pressure sensor by inhaling, presses a switch), the atomizer is activated (e.g., the heater is powered) and the liquid BCP composition is atomized or vaporized.

Depending on the embodiment and/or implementation, the heater and/or liquid BCP composition can reach a temperature from about 75° C. to about 350° C., from about 90° C. to about 300° C., from about 100° C. to about 290° C., from about 150° C. to about 280° C., from about 200° C. to about 275° C., from about 225° C. to about 270° C., from about 240° C. to about 265° C., or from about 250° C. to about 260° C. Some embodiments can include devices and/or configurations such as disclosed in one or more of the following, including combinations thereof: U.S. Pat. App. Pub. No. 2017/0023235; U.S. Pat. App. Pub. No. 2015/0114411; U.S. Pat. App. Pub. No. 2016/0000149; U.S. Pat. App. Pub. No. 2016/0270451; U.S. Pat. App. Pub. No. 2016/0053988; U.S. Pat. App. Pub. No. 2015/0327595; U.S. Pat. App. Pub. No. 2014/0299125; U.S. Pat. App. Pub. No. 2014/0182608; U.S. Pat. App. Pub. No. 2014/0286630; WIPO Patent Application Publication WO/2016/156497; WIPO Patent Application Publication WO/2016/030369; U.S. Pat. Nos. 9,320,300; 9,095,175; 9,259,035; 9,326,549; 7,997,280; 9,459,021; 9,427,711; and/or U.S. Pat. No. 9,352,288; each of which is expressly incorporated by reference herein for all purposes.

In some embodiments, the atomizer of a BCP device of the disclosure is configured to vaporize an average dose per actuation such that an average dose can be inhaled by a single inhalation of a user.

### Methods of Treatment

In some embodiments, the present disclosure teaches methods of treating a patient in need thereof with one or more of the vaporizable compositions of the present disclosure.

According to some embodiments, the compositions and methods of treatments disclosed herein are useful for treating one or more of dystonia, akathisia (as an anti-convulsant); glaucoma; ischemic disease (Alzheimer's, Parkinson's, Down Syndrome, HIV, dementia, etc.); for patients treated with oxidant-inducing agents for chemotherapy, radiation; motion sickness (anti-emetic); pain; pain and inflammation (e.g., arthritis, etc.); seizure disorders; neurological pain (e.g., MS-related); weight-loss; depression, attention (e.g., attention deficit disorder, attention deficit hyperactivity disorder), and other psychiatric disorders; sleep disorders (e.g., insomnia, sleep apnea, Wittmaack Ekbom's Syndrome, nightmares); sleepiness; smoking habits, nicotine habits, and/or other addictions/addictive behaviors. In some embodiments, a user of inhaled low-dose BCP can feel increased energy and can take the inhaled low-dose BCP in place of a cigarette or nicotine.

In some embodiments, the compositions, devices, and methods of treatments disclosed herein are useful in the treatment of at least one positive or negative symptom of one or more of dystonia, akathisia (anti-convulsant); glaucoma; ischemic disease (Alzheimer's, Parkinson's, Down Syndrome, HIV, dementia, etc.); for patients treated with oxidant-inducing agents for chemotherapy, radiation; motion sickness (anti-emetic); pain (chronic and acute); pain and inflammation (e.g., arthritis, etc.); seizure disorders; neurological pain (e.g., MS-related); weight-loss; depression, attention (e.g., attention deficit disorder, attention deficit hyperactivity disorder), and other psychiatric disorders; sleep disorders (e.g., insomnia, sleep apnea, Wittmaack Ekbom's Syndrome, nightmares); sleepiness; smoking habits, nicotine habits, and/or other addictions/addictive behaviors. The duration of treatment according to the methods of treatment of the disclosure can be any suitable duration as determined by a supervising health-care professional.

In some embodiments, the present invention is based on the inventor's discovery that the vaporizable compositions of the present disclosure produce unique and unexpected medical and recreational benefits not previously recognized for the ingestible or intravenous formulations of caryophyllene compounds. Thus in some embodiments, administration of the vaporizable compositions of the present disclosure results in increased energy, increased relaxation and drowsiness, and increased appetite.

In some embodiments, the present disclosure teaches methods of treating patients exhibiting loss of appetite, said method comprising administering one or more vaporizable compositions of the present disclosure to a patient in need thereof. In some embodiments, the vaporizable compositions can be used to treat anorexia, or other appetite loss symptoms stemming from AIDS, chemotherapy, or other medical conditions.

In some embodiments, the present disclosure teaches methods of treating sleep deprivation, said method comprising administering one or more vaporizable compositions of the present disclosure to a patient in need thereof. In some embodiments, the vaporizable compositions of the present disclosure can be used to treat insomnia, or other lack of sleep symptoms stemming from one or more medical conditions.

The present disclosure also teaches that vaporizable compositions of the invention also exhibit psychoactive effects on users. That is, in some embodiments, the administration of one or more of the vaporizable compositions of the present disclosure causes the patient to feel a “high,” despite the lack of any known psychoactive compound (e.g., despite complete lack of THC). Thus, the vaporizable compositions of the present disclosure produce a pleasurable psychoactive response that can result in medical and recreational benefits for the user.

Thus, in some embodiments, a patient receiving treatment for any one of the conditions described in this application would further experience a relaxing high with each dosage. This unexpected and pleasurable experience can improve patient adherence to treatment schedules, and can increase overall patient mood and outcomes.

Orally administered caryophyllene has also been associated with several health benefits, including anti-inflammatory properties. Caryophyllene is a food additive that is generally considered as safe for human consumption by the Food and Drug administration. The heretofore unknown psychoactive effects of the vaporizable compositions, together with their broad safety profile as recognized by the FDA, make the compositions a potentially valuable dietary supplement with recreational applications, beyond those of any previously disclosed oral doses.

As a result of the present invention, vaporizable compositions (e.g. BCP vaporizable compositions) can be used as a physician-recommended form of dietary supplement or holistic natural remedy without the side effects, or legal restrictions of traditional recreational marijuana plants and extracts.

As used herein, the term low dose is used to distinguish the presently disclosed doses from the oral administration doses of the prior art, which generally required administrations of greater than 500 mg of caryophyllene in order to exhibit any medical benefits. In some embodiments, the administration of the vaporizable compositions of the present disclosure provide medical benefits at a dose of about 0.75 mg, about 0.15 mg, about 0.075 mg, about 0.030 mg, or about 0.015 mg of BCP.

Beta Caryophyllene

According to some embodiments of the disclosure, there is provided vaporizable BCP compositions for use in a human subject in need thereof.

According to an aspect of some embodiments of the disclosure, there is also provided a device configured to provide vaporized BCP as a medicament, and in particular, a device configured to provide vaporized BCP as a treatment for a variety of conditions in a subject in need thereof.

In some embodiments, such a BCP composition is configured for administration to a human subject via inhalation. In some embodiments, such a BCP composition is configured for administration via aerosolization. In some embodiments, such a BCP composition is configured for administration via heat-based vaporization.

According to an aspect of some embodiments of the disclosure, there is also provided a method for treating a variety of conditions in a human subject in need thereof, the method comprising administering an aerosolized BCP composition to the subject via inhalation. In certain embodiments, the aerosolized BCP composition is a low-dose composition.

The efficacy of the methods and compositions according to the teachings of the disclosure are discussed in the experimental section.

According to some embodiments, the compositions and methods of treatments disclosed herein are useful for treating one or more of dystonia, akathisia (as an anti-convulsant); glaucoma; ischemic disease (Alzheimer's, Parkinson's, Down Syndrome, HIV, dementia, etc.); for patients treated with oxidant-inducing agents for chemotherapy, radiation; motion sickness (anti-emetic); pain; pain and inflammation (e.g., arthritis, etc.); seizure disorders; neurological pain (e.g., MS-related); weight-loss; depression, attention (e.g., attention deficit disorder, attention deficit hyperactivity disorder), and other psychiatric disorders; sleep disorders (e.g., insomnia, sleep apnea, Wittmaack Ekbom's Syndrome, nightmares); sleepiness; smoking habits, nicotine habits, and/or other addictions/addictive behaviors. For example, a user of inhaled low-dose BCP can feel increased energy and can take the inhaled low-dose BCP in place of a cigarette or nicotine. In some embodiments, the compositions, devices, and methods of treatments disclosed herein are useful in the treatment of at least one positive or negative symptom of one or more of dystonia, akathisia (anti-convulsant); glaucoma; ischemic disease (Alzheimer's, Parkinson's, Down Syndrome, HIV, dementia, etc.); for patients treated with oxidant-inducing agents for chemotherapy, radiation; motion sickness (anti-emetic); pain (chronic and acute); pain and inflammation (e.g., arthritis, etc.); seizure disorders; neurological pain (e.g., MS-related); weight-loss; depression, attention (e.g., attention deficit disorder, attention deficit hyperactivity disorder), and other psychiatric disorders; sleep disorders (e.g., insomnia, sleep apnea, Wittmaack Ekbom's Syndrome, nightmares); sleepiness; smoking habits, nicotine habits, and/or other addictions/addictive behaviors. The duration of treatment according to the methods of treatment of the disclosure can be any suitable duration as determined by a supervising health-care professional.

In some embodiments, a vaporizable BCP composition according to the disclosure is provided as or made as an inhalable dosage form.

In some embodiments of a method of the disclosure, the average dose of BCP administered to a human subject via inhalation of a vapor thereof is from about 0.005 mg/kg to about 0.350 mg/kg, such as, for example, from about 0.006 mg/kg to about 0.340 mg/kg, from about 0.007 mg/kg to about 0.335 mg/kg, from about 0.008 mg/kg to about 0.330 mg/kg, from about 0.009 mg/kg to about 0.325 mg/kg, from about 0.01 mg/kg to about 0.320 mg/kg, from about 0.011 mg/kg to about 0.315 mg/kg, from about 0.012 mg/kg to about 0.310 mg/kg, from about 0.013 mg/kg to about 0.305 mg/kg, from about 0.014 mg/kg to about 0.300 mg/kg, from about 0.015 mg/kg to about 0.295 mg/kg, from about 0.016 mg/kg to about 0.290 mg/kg, from about 0.017 mg/kg to about 0.285 mg/kg, from about 0.018 mg/kg to about 0.280 mg/kg, from about 0.019 mg/kg to about 0.275 mg/kg, from about 0.020 mg/kg to about 0.270 mg/kg, from about 0.021 mg/kg to about 0.265 mg/kg, from about 0.022 mg/kg to about 0.260 mg/kg, from about 0.023 mg/kg to about 0.255 mg/kg, from about 0.024 mg/kg to about 0.250 mg/kg, from about 0.025 mg/kg to about 0.245 mg/kg, from about 0.026 mg/kg to about 0.240 mg/kg, from about 0.027 mg/kg to about 0.235 mg/kg, from about 0.028 mg/kg to about 0.230 mg/kg, from about 0.029 mg/kg to about 0.225 mg/kg, from about 0.030 mg/kg to about 0.220 mg/kg, from about 0.031 mg/kg to about 0.215 mg/kg, from about 0.032 mg/kg to about 0.210 mg/kg, from about 0.033 mg/kg to about 0.205 mg/kg, from about 0.034 mg/kg to about 0.200 mg/kg, from about 0.035 mg/kg to about 0.195 mg/kg, from about 0.036 mg/kg to about 0.190 mg/kg, from about 0.037 mg/kg to about 0.185 mg/kg, from about 0.038 mg/kg to about 0.180 mg/kg, from about 0.039 mg/kg to about 0.175 mg/kg, from about 0.040 mg/kg to about 0.170 mg/kg, from about 0.045 mg/kg to about 0.165 mg/kg, from about 0.050 mg/kg to about 0.160 mg/kg, from about 0.055 mg/kg to about 0.155 mg/kg, from about 0.060 mg/kg to about 0.150 mg/kg, from about 0.065 mg/kg to about 0.145 mg/kg, from about 0.070 mg/kg to about 0.140 mg/kg, from about 0.075 mg/kg to about 0.135 mg/kg, from about 0.080 mg/kg to about 0.130 mg/kg, from about 0.085 mg/kg to about 0.125 mg/kg, from about 0.090 mg/kg to about 0.120 mg/kg, from about 0.095 mg/kg to about 0.115 mg/kg, or from about 0.100 mg/kg to about 0.110 mg/kg,

In some embodiments, the average dose of BCP is about 0.001 mg, about 0.005 mg, about 0.01 mg, about 0.015 mg, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.10 mg, about 0.105 mg, about 0.11 mg, about 0.115 mg, about 0.12 mg, about 0.125 mg, about 0.13 mg, about 0.135 mg, about 0.14 mg, about 0.145 mg, about 0.15 mg, about 0.155 mg, about 0.16 mg, about 0.165 mg, about 0.17 mg, about 0.175 mg, about 0.18 mg, about 0.185 mg, about 0.19 mg, about 0.195 mg, about 0.20 mg, about 0.205 mg, about 0.21 mg, about 0.215 mg, about 0.22 mg, about 0.225 mg, about 0.23 mg, about 0.235 mg, about 0.24 mg, about 0.245 mg, about 0.25 mg, about 0.255 mg, about 0.26 mg, about 0.265 mg, about 0.27 mg, about 0.275 mg, about 0.28 mg, about 0.285 mg, about 0.29 mg, about 0.295 mg, about 0.30 mg, about 0.305 mg, about 0.31 mg, about 0.315 mg, about 0.32 mg, about 0.325 mg, about 0.33 mg, about 0.335 mg, about 0.34 mg, about 0.345 mg, about 0.35 mg, about 0.355 mg, about 0.36 mg, about 0.365 mg, about 0.37 mg, about 0.375 mg, about 0.38 mg, about 0.385 mg, about 0.39 mg, about 0.395 mg, about 0.40 mg, about 0.405 mg, about 0.41 mg, about 0.415 mg, about 0.42 mg, about 0.425 mg, about 0.43 mg, about 0.435 mg, about 0.44 mg, about 0.445 mg, about 0.45 mg, about 0.455 mg, about 0.46 mg, about 0.465 mg, about 0.47 mg, about 0.475 mg, about 0.48 mg, about 0.485 mg, about 0.49 mg, about 0.495 mg, about 0.50 mg, about 0.505 mg, about 0.51 mg, about 0.515 mg, about 0.52 mg, about 0.525 mg, about 0.53 mg, about 0.535 mg, about 0.54 mg, about 0.545 mg, about 0.55 mg, about 0.555 mg, about 0.56 mg, about 0.565 mg, about 0.57 mg, about 0.575 mg, about 0.58 mg, about 0.585 mg, about 0.59 mg, about 0.595 mg, about 0.60 mg, about 0.605 mg, about 0.61 mg, about 0.615 mg, about 0.62 mg, about 0.625 mg, about 0.63 mg, about 0.635 mg, about 0.64 mg, about 0.645 mg, about 0.65 mg, about 0.655 mg, about 0.66 mg, about 0.665 mg, about 0.67 mg, about 0.675 mg, about 0.68 mg, about 0.685 mg, about 0.69 mg, about 0.695 mg, about 0.70 mg, about 0.705 mg, about 0.71 mg, about 0.715 mg, about 0.72 mg, about 0.725 mg, about 0.73 mg, about 0.735 mg, about 0.74 mg, about 0.745 mg, about 0.75 mg, about 0.755 mg, about 0.76 mg, about 0.765 mg, about 0.77 mg, about 0.775 mg, about 0.78 mg, about 0.785 mg, about 0.79 mg, about 0.795 mg, about 0.80 mg, about 0.805 mg, about 0.81 mg, about 0.815 mg, about 0.82 mg, about 0.825 mg, about 0.83 mg, about 0.835 mg, about 0.84 mg, about 0.845 mg, about 0.85 mg, about 0.855 mg, about 0.86 mg, about 0.865 mg, about 0.87 mg, about 0.875 mg, about 0.88 mg, about 0.885 mg, about 0.89 mg, about 0.895 mg, about 0.90 mg, about 0.905 mg, about 0.91 mg, about 0.915 mg, about 0.92 mg, about 0.925 mg, about 0.93 mg, about 0.935 mg, about 0.94 mg, about 0.945 mg, about 0.95 mg, about 0.955 mg, about 0.96 mg, about 0.965 mg, about 0.97 mg, about 0.975 mg, about 0.98 mg, about 0.985 mg, about 0.99 mg, or about 0.995 mg.

In some embodiments, the average dose of BCP is about 1.00 mg, about, about 1.005 mg, about 1.01 mg, about 1.015 mg, about 1.02 mg, about 1.025 mg, about 1.03 mg, about 1.035 mg, about 1.04 mg, about 1.045 mg, about 1.05 mg, about 1.055 mg, about 1.06 mg, about 1.065 mg, about 1.07 mg, about 1.075 mg, about 1.08 mg, about 1.085 mg, about 1.09 mg, about 1.095 mg, about 1.10 mg, about 1.105 mg, about 1.11 mg, about 1.115 mg, about 1.12 mg, about 1.125 mg, about 1.13 mg, about 1.135 mg, about 1.14 mg, about 1.145 mg, about 1.15 mg, about 1.155 mg, about 1.16 mg, about 1.165 mg, about 1.17 mg, about 1.175 mg, about 1.18 mg, about 1.185 mg, about 1.19 mg, about 1.195 mg, about 1.20 mg, about 1.205 mg, about 1.21 mg, about 1.215 mg, about 1.22 mg, about 1.225 mg, about 1.23 mg, about 1.235 mg, about 1.24 mg, about 1.245 mg, about 1.25 mg, about 1.255 mg, about 1.26 mg, about 1.265 mg, about 1.27 mg, about 1.275 mg, about 1.28 mg, about 1.285 mg, about 1.29 mg, about 1.295 mg, about 1.30 mg, about 1.305 mg, about 1.31 mg, about 1.315 mg, about 1.32 mg, about 1.325 mg, about 1.33 mg, about 1.335 mg, about 1.34 mg, about 1.345 mg, about 1.35 mg, about 1.355 mg, about 1.36 mg, about 1.365 mg, about 1.37 mg, about 1.375 mg, about 1.38 mg, about 1.385 mg, about 1.39 mg, about 1.395 mg, about 1.40 mg, about 1.405 mg, about 1.41 mg, about 1.415 mg, about 1.42 mg, about 1.425 mg, about 1.43 mg, about 1.435 mg, about 1.44 mg, about 1.445 mg, about 1.45 mg, about 1.455 mg, about 1.46 mg, about 1.465 mg, about 1.47 mg, about 1.475 mg, about 1.48 mg, about 1.485 mg, about 1.49 mg, about 1.495 mg, about 1.50 mg, about 1.505 mg, about 1.51 mg, about 1.515 mg, about 1.52 mg, about 1.525 mg, about 1.53 mg, about 1.535 mg, about 1.54 mg, about 1.545 mg, about 1.55 mg, about 1.555 mg, about 1.56 mg, about 1.565 mg, about 1.57 mg, about 1.575 mg, about 1.58 mg, about 1.585 mg, about 1.59 mg, about 1.595 mg, about 1.60 mg, about 1.605 mg, about 1.61 mg, about 1.615 mg, about 1.62 mg, about 1.625 mg, about 1.63 mg, about 1.635 mg, about 1.64 mg, about 1.645 mg, about 1.65 mg, about 1.655 mg, about 1.66 mg, about 1.665 mg, about 1.67 mg, about 1.675 mg, about 1.68 mg, about 1.685 mg, about 1.69 mg, about 1.695 mg, about 1.70 mg, about 1.705 mg, about 1.71 mg, about 1.715 mg, about 1.72 mg, about 1.725 mg, about 1.73 mg, about 1.735 mg, about 1.74 mg, about 1.745 mg, about 1.75 mg, about 1.755 mg, about 1.76 mg, about 1.765 mg, about 1.77 mg, about 1.775 mg, about 1.78 mg, about 1.785 mg, about 1.79 mg, about 1.795 mg, about 1.80 mg, about 1.805 mg, about 1.81 mg, about 1.815 mg, about 1.82 mg, about 1.825 mg, about 1.83 mg, about 1.835 mg, about 1.84 mg, about 1.845 mg, about 1.85 mg, about 1.855 mg, about 1.86 mg, about 1.865 mg, about 1.87 mg, about 1.875 mg, about 1.88 mg, about 1.885 mg, about 1.89 mg, about 1.895 mg, about 1.90 mg, about 1.905 mg, about 1.91 mg, about 1.915 mg, about 1.92 mg, about 1.925 mg, about 1.93 mg, about 1.935 mg, about 1.94 mg, about 1.945 mg, about 1.95 mg, about 1.955 mg, about 1.96 mg, about 1.965 mg, about 1.97 mg, about 1.975 mg, about 1.98 mg, about 1.985 mg, about 1.99 mg, or about 1.995 mg.

In some embodiments, the average dose of BCP is about 2.00 mg, about, about 2.005 mg, about 2.01 mg, about 2.015 mg, about 2.02 mg, about 2.025 mg, about 2.03 mg, about 2.035 mg, about 2.04 mg, about 2.045 mg, about 2.05 mg, about 2.055 mg, about 2.06 mg, about 2.065 mg, about 2.07 mg, about 2.075 mg, about 2.08 mg, about 2.085 mg, about 2.09 mg, about 2.095 mg, about 2.10 mg, about 2.105 mg, about 2.11 mg, about 2.115 mg, about 2.12 mg, about 2.125 mg, about 2.13 mg, about 2.135 mg, about 2.14 mg, about 2.145 mg, about 2.15 mg, about 2.155 mg, about 2.16 mg, about 2.165 mg, about 2.17 mg, about 2.175 mg, about 2.18 mg, about 2.185 mg, about 2.19 mg, about 2.195 mg, about 2.20 mg, about 2.205 mg, about 2.21 mg, about 2.215 mg, about 2.22 mg, about 2.225 mg, about 2.23 mg, about 2.235 mg, about 2.24 mg, about 2.245 mg, about 2.25 mg, about 2.255 mg, about 2.26 mg, about 2.265 mg, about 2.27 mg, about 2.275 mg, about 2.28 mg, about 2.285 mg, about 2.29 mg, about 2.295 mg, about 2.30 mg, about 2.305 mg, about 2.31 mg, about 2.315 mg, about 2.32 mg, about 2.325 mg, about 2.33 mg, about 2.335 mg, about 2.34 mg, about 2.345 mg, about 2.35 mg, about 2.355 mg, about 2.36 mg, about 2.365 mg, about 2.37 mg, about 2.375 mg, about 2.38 mg, about 2.385 mg, about 2.39 mg, about 2.395 mg, about 2.40 mg, about 2.405 mg, about 2.41 mg, about 2.415 mg, about 2.42 mg, about 2.425 mg, about 2.43 mg, about 2.435 mg, about 2.44 mg, about 2.445 mg, about 2.45 mg, about 2.455 mg, about 2.46 mg, about 2.465 mg, about 2.47 mg, about 2.475 mg, about 2.48 mg, about 2.485 mg, about 2.49 mg, about 2.495 mg, about 2.50 mg, about 2.505 mg, about 2.51 mg, about 2.515 mg, about 2.52 mg, about 2.525 mg, about 2.53 mg, about 2.535 mg, about 2.54 mg, about 2.545 mg, about 2.55 mg, about 2.555 mg, about 2.56 mg, about 2.565 mg, about 2.57 mg, about 2.575 mg, about 2.58 mg, about 2.585 mg, about 2.59 mg, about 2.595 mg, about 2.60 mg, about 2.605 mg, about 2.61 mg, about 2.615 mg, about 2.62 mg, about 2.625 mg, about 2.63 mg, about 2.635 mg, about 2.64 mg, about 2.645 mg, about 2.65 mg, about 2.655 mg, about 2.66 mg, about 2.665 mg, about 2.67 mg, about 2.675 mg, about 2.68 mg, about 2.685 mg, about 2.69 mg, about 2.695 mg, about 2.70 mg, about 2.705 mg, about 2.71 mg, about 2.715 mg, about 2.72 mg, about 2.725 mg, about 2.73 mg, about 2.735 mg, about 2.74 mg, about 2.745 mg, about 2.75 mg, about 2.755 mg, about 2.76 mg, about 2.765 mg, about 2.77 mg, about 2.775 mg, about 2.78 mg, about 2.785 mg, about 2.79 mg, about 2.795 mg, about 2.80 mg, about 2.805 mg, about 2.81 mg, about 2.815 mg, about 2.82 mg, about 2.825 mg, about 2.83 mg, about 2.835 mg, about 2.84 mg, about 2.845 mg, about 2.85 mg, about 2.855 mg, about 2.86 mg, about 2.865 mg, about 2.87 mg, about 2.875 mg, about 2.88 mg, about 2.885 mg, about 2.89 mg, about 2.895 mg, about 2.90 mg, about 2.905 mg, about 2.91 mg, about 2.915 mg, about 2.92 mg, about 2.925 mg, about 2.93 mg, about 2.935 mg, about 2.94 mg, about 2.945 mg, about 2.95 mg, about 2.955 mg, about 2.96 mg, about 2.965 mg, about 2.97 mg, about 2.975 mg, about 2.98 mg, about 2.985 mg, about 2.99 mg, or about 2.995 mg.

In some embodiments, the average dose of BCP is about 3.00 mg, about, about 3.005 mg, about 3.01 mg, about 3.015 mg, about 3.02 mg, about 3.025 mg, about 3.03 mg, about 3.035 mg, about 3.04 mg, about 3.045 mg, about 3.05 mg, about 3.055 mg, about 3.06 mg, about 3.065 mg, about 3.07 mg, about 3.075 mg, about 3.08 mg, about 3.085 mg, about 3.09 mg, about 3.095 mg, about 3.10 mg, about 3.105 mg, about 3.11 mg, about 3.115 mg, about 3.12 mg, about 3.125 mg, about 3.13 mg, about 3.135 mg, about 3.14 mg, about 3.145 mg, about 3.15 mg, about 3.155 mg, about 3.16 mg, about 3.165 mg, about 3.17 mg, about 3.175 mg, about 3.18 mg, about 3.185 mg, about 3.19 mg, about 3.195 mg, about 3.20 mg, about 3.205 mg, about 3.21 mg, about 3.215 mg, about 3.22 mg, about 3.225 mg, about 3.23 mg, about 3.235 mg, about 3.24 mg, about 3.245 mg, about 3.25 mg, about 3.255 mg, about 3.26 mg, about 3.265 mg, about 3.27 mg, about 3.275 mg, about 3.28 mg, about 3.285 mg, about 3.29 mg, about 3.295 mg, about 3.30 mg, about 3.305 mg, about 3.31 mg, about 3.315 mg, about 3.32 mg, about 3.325 mg, about 3.33 mg, about 3.335 mg, about 3.34 mg, about 3.345 mg, about 3.35 mg, about 3.355 mg, about 3.36 mg, about 3.365 mg, about 3.37 mg, about 3.375 mg, about 3.38 mg, about 3.385 mg, about 3.39 mg, about 3.395 mg, about 3.40 mg, about 3.405 mg, about 3.41 mg, about 3.415 mg, about 3.42 mg, about 3.425 mg, about 3.43 mg, about 3.435 mg, about 3.44 mg, about 3.445 mg, about 3.45 mg, about 3.455 mg, about 3.46 mg, about 3.465 mg, about 3.47 mg, about 3.475 mg, about 3.48 mg, about 3.485 mg, about 3.49 mg, about 3.495 mg, about 3.50 mg, about 3.505 mg, about 3.51 mg, about 3.515 mg, about 3.52 mg, about 3.525 mg, about 3.53 mg, about 3.535 mg, about 3.54 mg, about 3.545 mg, about 3.55 mg, about 3.555 mg, about 3.56 mg, about 3.565 mg, about 3.57 mg, about 3.575 mg, about 3.58 mg, about 3.585 mg, about 3.59 mg, about 3.595 mg, about 3.60 mg, about 3.605 mg, about 3.61 mg, about 3.615 mg, about 3.62 mg, about 3.625 mg, about 3.63 mg, about 3.635 mg, about 3.64 mg, about 3.645 mg, about 3.65 mg, about 3.655 mg, about 3.66 mg, about 3.665 mg, about 3.67 mg, about 3.675 mg, about 3.68 mg, about 3.685 mg, about 3.69 mg, about 3.695 mg, about 3.70 mg, about 3.705 mg, about 3.71 mg, about 3.715 mg, about 3.72 mg, about 3.725 mg, about 3.73 mg, about 3.735 mg, about 3.74 mg, about 3.745 mg, about 3.75 mg, about 3.755 mg, about 3.76 mg, about 3.765 mg, about 3.77 mg, about 3.775 mg, about 3.78 mg, about 3.785 mg, about 3.79 mg, about 3.795 mg, about 3.80 mg, about 3.805 mg, about 3.81 mg, about 3.815 mg, about 3.82 mg, about 3.825 mg, about 3.83 mg, about 3.835 mg, about 3.84 mg, about 3.845 mg, about 3.85 mg, about 3.855 mg, about 3.86 mg, about 3.865 mg, about 3.87 mg, about 3.875 mg, about 3.88 mg, about 3.885 mg, about 3.89 mg, about 3.895 mg, about 3.90 mg, about 3.905 mg, about 3.91 mg, about 3.915 mg, about 3.92 mg, about 3.925 mg, about 3.93 mg, about 3.935 mg, about 3.94 mg, about 3.945 mg, about 3.95 mg, about 3.955 mg, about 3.96 mg, about 3.965 mg, about 3.97 mg, about 3.975 mg, about 3.98 mg, about 3.985 mg, about 3.99 mg, or about 3.995 mg.

In some embodiments, the average dose of BCP is about 4.00 mg, about, about 4.005 mg, about 4.01 mg, about 4.015 mg, about 4.02 mg, about 4.025 mg, about 4.03 mg, about 4.035 mg, about 4.04 mg, about 4.045 mg, about 4.05 mg, about 4.055 mg, about 4.06 mg, about 4.065 mg, about 4.07 mg, about 4.075 mg, about 4.08 mg, about 4.085 mg, about 4.09 mg, about 4.095 mg, about 4.10 mg, about 4.105 mg, about 4.11 mg, about 4.115 mg, about 4.12 mg, about 4.125 mg, about 4.13 mg, about 4.135 mg, about 4.14 mg, about 4.145 mg, about 4.15 mg, about 4.155 mg, about 4.16 mg, about 4.165 mg, about 4.17 mg, about 4.175 mg, about 4.18 mg, about 4.185 mg, about 4.19 mg, about 4.195 mg, about 4.20 mg, about 4.205 mg, about 4.21 mg, about 4.215 mg, about 4.22 mg, about 4.225 mg, about 4.23 mg, about 4.235 mg, about 4.24 mg, about 4.245 mg, about 4.25 mg, about 4.255 mg, about 4.26 mg, about 4.265 mg, about 4.27 mg, about 4.275 mg, about 4.28 mg, about 4.285 mg, about 4.29 mg, about 4.295 mg, about 4.30 mg, about 4.305 mg, about 4.31 mg, about 4.315 mg, about 4.32 mg, about 4.325 mg, about 4.33 mg, about 4.335 mg, about 4.34 mg, about 4.345 mg, about 4.35 mg, about 4.355 mg, about 4.36 mg, about 4.365 mg, about 4.37 mg, about 4.375 mg, about 4.38 mg, about 4.385 mg, about 4.39 mg, about 4.395 mg, about 4.40 mg, about 4.405 mg, about 4.41 mg, about 4.415 mg, about 4.42 mg, about 4.425 mg, about 4.43 mg, about 4.435 mg, about 4.44 mg, about 4.445 mg, about 4.45 mg, about 4.455 mg, about 4.46 mg, about 4.465 mg, about 4.47 mg, about 4.475 mg, about 4.48 mg, about 4.485 mg, about 4.49 mg, about 4.495 mg, about 4.50 mg, about 4.505 mg, about 4.51 mg, about 4.515 mg, about 4.52 mg, about 4.525 mg, about 4.53 mg, about 4.535 mg, about 4.54 mg, about 4.545 mg, about 4.55 mg, about 4.555 mg, about 4.56 mg, about 4.565 mg, about 4.57 mg, about 4.575 mg, about 4.58 mg, about 4.585 mg, about 4.59 mg, about 4.595 mg, about 4.60 mg, about 4.605 mg, about 4.61 mg, about 4.615 mg, about 4.62 mg, about 4.625 mg, about 4.63 mg, about 4.635 mg, about 4.64 mg, about 4.645 mg, about 4.65 mg, about 4.655 mg, about 4.66 mg, about 4.665 mg, about 4.67 mg, about 4.675 mg, about 4.68 mg, about 4.685 mg, about 4.69 mg, about 4.695 mg, about 4.70 mg, about 4.705 mg, about 4.71 mg, about 4.715 mg, about 4.72 mg, about 4.725 mg, about 4.73 mg, about 4.735 mg, about 4.74 mg, about 4.745 mg, about 4.75 mg, about 4.755 mg, about 4.76 mg, about 4.765 mg, about 4.77 mg, about 4.775 mg, about 4.78 mg, about 4.785 mg, about 4.79 mg, about 4.795 mg, about 4.80 mg, about 4.805 mg, about 4.81 mg, about 4.815 mg, about 4.82 mg, about 4.825 mg, about 4.83 mg, about 4.835 mg, about 4.84 mg, about 4.845 mg, about 4.85 mg, about 4.855 mg, about 4.86 mg, about 4.865 mg, about 4.87 mg, about 4.875 mg, about 4.88 mg, about 4.885 mg, about 4.89 mg, about 4.895 mg, about 4.90 mg, about 4.905 mg, about 4.91 mg, about 4.915 mg, about 4.92 mg, about 4.925 mg, about 4.93 mg, about 4.935 mg, about 4.94 mg, about 4.945 mg, about 4.95 mg, about 4.955 mg, about 4.96 mg, about 4.965 mg, about 4.97 mg, about 4.975 mg, about 4.98 mg, about 4.985 mg, about 4.99 mg, or about 4.995 mg.

In some embodiments, the average dose of BCP is about 5.00 mg, about, about 5.005 mg, about 5.01 mg, about 5.015 mg, about 5.02 mg, about 5.025 mg, about 5.03 mg, about 5.035 mg, about 5.04 mg, about 5.045 mg, about 5.05 mg, about 5.055 mg, about 5.06 mg, about 5.065 mg, about 5.07 mg, about 5.075 mg, about 5.08 mg, about 5.085 mg, about 5.09 mg, about 5.095 mg, about 5.10 mg, about 5.105 mg, about 5.11 mg, about 5.115 mg, about 5.12 mg, about 5.125 mg, about 5.13 mg, about 5.135 mg, about 5.14 mg, about 5.145 mg, about 5.15 mg, about 5.155 mg, about 5.16 mg, about 5.165 mg, about 5.17 mg, about 5.175 mg, about 5.18 mg, about 5.185 mg, about 5.19 mg, about 5.195 mg, about 5.20 mg, about 5.205 mg, about 5.21 mg, about 5.215 mg, about 5.22 mg, about 5.225 mg, about 5.23 mg, about 5.235 mg, about 5.24 mg, about 5.245 mg, about 5.25 mg, about 5.255 mg, about 5.26 mg, about 5.265 mg, about 5.27 mg, about 5.275 mg, about 5.28 mg, about 5.285 mg, about 5.29 mg, about 5.295 mg, about 5.30 mg, about 5.305 mg, about 5.31 mg, about 5.315 mg, about 5.32 mg, about 5.325 mg, about 5.33 mg, about 5.335 mg, about 5.34 mg, about 5.345 mg, about 5.35 mg, about 5.355 mg, about 5.36 mg, about 5.365 mg, about 5.37 mg, about 5.375 mg, about 5.38 mg, about 5.385 mg, about 5.39 mg, about 5.395 mg, about 5.40 mg, about 5.405 mg, about 5.41 mg, about 5.415 mg, about 5.42 mg, about 5.425 mg, about 5.43 mg, about 5.435 mg, about 5.44 mg, about 5.445 mg, about 5.45 mg, about 5.455 mg, about 5.46 mg, about 5.465 mg, about 5.47 mg, about 5.475 mg, about 5.48 mg, about 5.485 mg, about 5.49 mg, about 5.495 mg, about 5.50 mg, about 5.505 mg, about 5.51 mg, about 5.515 mg, about 5.52 mg, about 5.525 mg, about 5.53 mg, about 5.535 mg, about 5.54 mg, about 5.545 mg, about 5.55 mg, about 5.555 mg, about 5.56 mg, about 5.565 mg, about 5.57 mg, about 5.575 mg, about 5.58 mg, about 5.585 mg, about 5.59 mg, about 5.595 mg, about 5.60 mg, about 5.605 mg, about 5.61 mg, about 5.615 mg, about 5.62 mg, about 5.625 mg, about 5.63 mg, about 5.635 mg, about 5.64 mg, about 5.645 mg, about 5.65 mg, about 5.655 mg, about 5.66 mg, about 5.665 mg, about 5.67 mg, about 5.675 mg, about 5.68 mg, about 5.685 mg, about 5.69 mg, about 5.695 mg, about 5.70 mg, about 5.705 mg, about 5.71 mg, about 5.715 mg, about 5.72 mg, about 5.725 mg, about 5.73 mg, about 5.735 mg, about 5.74 mg, about 5.745 mg, about 5.75 mg, about 5.755 mg, about 5.76 mg, about 5.765 mg, about 5.77 mg, about 5.775 mg, about 5.78 mg, about 5.785 mg, about 5.79 mg, about 5.795 mg, about 5.80 mg, about 5.805 mg, about 5.81 mg, about 5.815 mg, about 5.82 mg, about 5.825 mg, about 5.83 mg, about 5.835 mg, about 5.84 mg, about 5.845 mg, about 5.85 mg, about 5.855 mg, about 5.86 mg, about 5.865 mg, about 5.87 mg, about 5.875 mg, about 5.88 mg, about 5.885 mg, about 5.89 mg, about 5.895 mg, about 5.90 mg, about 5.905 mg, about 5.91 mg, about 5.915 mg, about 5.92 mg, about 5.925 mg, about 5.93 mg, about 5.935 mg, about 5.94 mg, about 5.945 mg, about 5.95 mg, about 5.955 mg, about 5.96 mg, about 5.965 mg, about 5.97 mg, about 5.975 mg, about 5.98 mg, about 5.985 mg, about 5.99 mg, or about 5.995 mg.

In some embodiments, the average dose of BCP is about 6.00 mg, about, about 6.005 mg, about 6.01 mg, about 6.015 mg, about 6.02 mg, about 6.025 mg, about 6.03 mg, about 6.035 mg, about 6.04 mg, about 6.045 mg, about 6.05 mg, about 6.055 mg, about 6.06 mg, about 6.065 mg, about 6.07 mg, about 6.075 mg, about 6.08 mg, about 6.085 mg, about 6.09 mg, about 6.095 mg, about 6.10 mg, about 6.105 mg, about 6.11 mg, about 6.115 mg, about 6.12 mg, about 6.125 mg, about 6.13 mg, about 6.135 mg, about 6.14 mg, about 6.145 mg, about 6.15 mg, about 6.155 mg, about 6.16 mg, about 6.165 mg, about 6.17 mg, about 6.175 mg, about 6.18 mg, about 6.185 mg, about 6.19 mg, about 6.195 mg, about 6.20 mg, about 6.205 mg, about 6.21 mg, about 6.215 mg, about 6.22 mg, about 6.225 mg, about 6.23 mg, about 6.235 mg, about 6.24 mg, about 6.245 mg, about 6.25 mg, about 6.255 mg, about 6.26 mg, about 6.265 mg, about 6.27 mg, about 6.275 mg, about 6.28 mg, about 6.285 mg, about 6.29 mg, about 6.295 mg, about 6.30 mg, about 6.305 mg, about 6.31 mg, about 6.315 mg, about 6.32 mg, about 6.325 mg, about 6.33 mg, about 6.335 mg, about 6.34 mg, about 6.345 mg, about 6.35 mg, about 6.355 mg, about 6.36 mg, about 6.365 mg, about 6.37 mg, about 6.375 mg, about 6.38 mg, about 6.385 mg, about 6.39 mg, about 6.395 mg, about 6.40 mg, about 6.405 mg, about 6.41 mg, about 6.415 mg, about 6.42 mg, about 6.425 mg, about 6.43 mg, about 6.435 mg, about 6.44 mg, about 6.445 mg, about 6.45 mg, about 6.455 mg, about 6.46 mg, about 6.465 mg, about 6.47 mg, about 6.475 mg, about 6.48 mg, about 6.485 mg, about 6.49 mg, about 6.495 mg, about 6.50 mg, about 6.505 mg, about 6.51 mg, about 6.515 mg, about 6.52 mg, about 6.525 mg, about 6.53 mg, about 6.535 mg, about 6.54 mg, about 6.545 mg, about 6.55 mg, about 6.555 mg, about 6.56 mg, about 6.565 mg, about 6.57 mg, about 6.575 mg, about 6.58 mg, about 6.585 mg, about 6.59 mg, about 6.595 mg, about 6.60 mg, about 6.605 mg, about 6.61 mg, about 6.615 mg, about 6.62 mg, about 6.625 mg, about 6.63 mg, about 6.635 mg, about 6.64 mg, about 6.645 mg, about 6.65 mg, about 6.655 mg, about 6.66 mg, about 6.665 mg, about 6.67 mg, about 6.675 mg, about 6.68 mg, about 6.685 mg, about 6.69 mg, about 6.695 mg, about 6.70 mg, about 6.705 mg, about 6.71 mg, about 6.715 mg, about 6.72 mg, about 6.725 mg, about 6.73 mg, about 6.735 mg, about 6.74 mg, about 6.745 mg, about 6.75 mg, about 6.755 mg, about 6.76 mg, about 6.765 mg, about 6.77 mg, about 6.775 mg, about 6.78 mg, about 6.785 mg, about 6.79 mg, about 6.795 mg, about 6.80 mg, about 6.805 mg, about 6.81 mg, about 6.815 mg, about 6.82 mg, about 6.825 mg, about 6.83 mg, about 6.835 mg, about 6.84 mg, about 6.845 mg, about 6.85 mg, about 6.855 mg, about 6.86 mg, about 6.865 mg, about 6.87 mg, about 6.875 mg, about 6.88 mg, about 6.885 mg, about 6.89 mg, about 6.895 mg, about 6.90 mg, about 6.905 mg, about 6.91 mg, about 6.915 mg, about 6.92 mg, about 6.925 mg, about 6.93 mg, about 6.935 mg, about 6.94 mg, about 6.945 mg, about 6.95 mg, about 6.955 mg, about 6.96 mg, about 6.965 mg, about 6.97 mg, about 6.975 mg, about 6.98 mg, about 6.985 mg, about 6.99 mg, or about 6.995 mg.

In some embodiments, the average dose of BCP is about 7.00 mg, about, about 7.005 mg, about 7.01 mg, about 7.015 mg, about 7.02 mg, about 7.025 mg, about 7.03 mg, about 7.035 mg, about 7.04 mg, about 7.045 mg, about 7.05 mg, about 7.055 mg, about 7.06 mg, about 7.065 mg, about 7.07 mg, about 7.075 mg, about 7.08 mg, about 7.085 mg, about 7.09 mg, about 7.095 mg, about 7.10 mg, about 7.105 mg, about 7.11 mg, about 7.115 mg, about 7.12 mg, about 7.125 mg, about 7.13 mg, about 7.135 mg, about 7.14 mg, about 7.145 mg, about 7.15 mg, about 7.155 mg, about 7.16 mg, about 7.165 mg, about 7.17 mg, about 7.175 mg, about 7.18 mg, about 7.185 mg, about 7.19 mg, about 7.195 mg, about 7.20 mg, about 7.205 mg, about 7.21 mg, about 7.215 mg, about 7.22 mg, about 7.225 mg, about 7.23 mg, about 7.235 mg, about 7.24 mg, about 7.245 mg, about 7.25 mg, about 7.255 mg, about 7.26 mg, about 7.265 mg, about 7.27 mg, about 7.275 mg, about 7.28 mg, about 7.285 mg, about 7.29 mg, about 7.295 mg, about 7.30 mg, about 7.305 mg, about 7.31 mg, about 7.315 mg, about 7.32 mg, about 7.325 mg, about 7.33 mg, about 7.335 mg, about 7.34 mg, about 7.345 mg, about 7.35 mg, about 7.355 mg, about 7.36 mg, about 7.365 mg, about 7.37 mg, about 7.375 mg, about 7.38 mg, about 7.385 mg, about 7.39 mg, about 7.395 mg, about 7.40 mg, about 7.405 mg, about 7.41 mg, about 7.415 mg, about 7.42 mg, about 7.425 mg, about 7.43 mg, about 7.435 mg, about 7.44 mg, about 7.445 mg, about 7.45 mg, about 7.455 mg, about 7.46 mg, about 7.465 mg, about 7.47 mg, about 7.475 mg, about 7.48 mg, about 7.485 mg, about 7.49 mg, about 7.495 mg, about 7.50 mg, about 7.505 mg, about 7.51 mg, about 7.515 mg, about 7.52 mg, about 7.525 mg, about 7.53 mg, about 7.535 mg, about 7.54 mg, about 7.545 mg, about 7.55 mg, about 7.555 mg, about 7.56 mg, about 7.565 mg, about 7.57 mg, about 7.575 mg, about 7.58 mg, about 7.585 mg, about 7.59 mg, about 7.595 mg, about 7.60 mg, about 7.605 mg, about 7.61 mg, about 7.615 mg, about 7.62 mg, about 7.625 mg, about 7.63 mg, about 7.635 mg, about 7.64 mg, about 7.645 mg, about 7.65 mg, about 7.655 mg, about 7.66 mg, about 7.665 mg, about 7.67 mg, about 7.675 mg, about 7.68 mg, about 7.685 mg, about 7.69 mg, about 7.695 mg, about 7.70 mg, about 7.705 mg, about 7.71 mg, about 7.715 mg, about 7.72 mg, about 7.725 mg, about 7.73 mg, about 7.735 mg, about 7.74 mg, about 7.745 mg, about 7.75 mg, about 7.755 mg, about 7.76 mg, about 7.765 mg, about 7.77 mg, about 7.775 mg, about 7.78 mg, about 7.785 mg, about 7.79 mg, about 7.795 mg, about 7.80 mg, about 7.805 mg, about 7.81 mg, about 7.815 mg, about 7.82 mg, about 7.825 mg, about 7.83 mg, about 7.835 mg, about 7.84 mg, about 7.845 mg, about 7.85 mg, about 7.855 mg, about 7.86 mg, about 7.865 mg, about 7.87 mg, about 7.875 mg, about 7.88 mg, about 7.885 mg, about 7.89 mg, about 7.895 mg, about 7.90 mg, about 7.905 mg, about 7.91 mg, about 7.915 mg, about 7.92 mg, about 7.925 mg, about 7.93 mg, about 7.935 mg, about 7.94 mg, about 7.945 mg, about 7.95 mg, about 7.955 mg, about 7.96 mg, about 7.965 mg, about 7.97 mg, about 7.975 mg, about 7.98 mg, about 7.985 mg, about 7.99 mg, or about 7.995 mg.

In some embodiments, the average dose of BCP is about 8.00 mg, about, about 8.005 mg, about 8.01 mg, about 8.015 mg, about 8.02 mg, about 8.025 mg, about 8.03 mg, about 8.035 mg, about 8.04 mg, about 8.045 mg, about 8.05 mg, about 8.055 mg, about 8.06 mg, about 8.065 mg, about 8.07 mg, about 8.075 mg, about 8.08 mg, about 8.085 mg, about 8.09 mg, about 8.095 mg, about 8.10 mg, about 8.105 mg, about 8.11 mg, about 8.115 mg, about 8.12 mg, about 8.125 mg, about 8.13 mg, about 8.135 mg, about 8.14 mg, about 8.145 mg, about 8.15 mg, about 8.155 mg, about 8.16 mg, about 8.165 mg, about 8.17 mg, about 8.175 mg, about 8.18 mg, about 8.185 mg, about 8.19 mg, about 8.195 mg, about 8.20 mg, about 8.205 mg, about 8.21 mg, about 8.215 mg, about 8.22 mg, about 8.225 mg, about 8.23 mg, about 8.235 mg, about 8.24 mg, about 8.245 mg, about 8.25 mg, about 8.255 mg, about 8.26 mg, about 8.265 mg, about 8.27 mg, about 8.275 mg, about 8.28 mg, about 8.285 mg, about 8.29 mg, about 8.295 mg, about 8.30 mg, about 8.305 mg, about 8.31 mg, about 8.315 mg, about 8.32 mg, about 8.325 mg, about 8.33 mg, about 8.335 mg, about 8.34 mg, about 8.345 mg, about 8.35 mg, about 8.355 mg, about 8.36 mg, about 8.365 mg, about 8.37 mg, about 8.375 mg, about 8.38 mg, about 8.385 mg, about 8.39 mg, about 8.395 mg, about 8.40 mg, about 8.405 mg, about 8.41 mg, about 8.415 mg, about 8.42 mg, about 8.425 mg, about 8.43 mg, about 8.435 mg, about 8.44 mg, about 8.445 mg, about 8.45 mg, about 8.455 mg, about 8.46 mg, about 8.465 mg, about 8.47 mg, about 8.475 mg, about 8.48 mg, about 8.485 mg, about 8.49 mg, about 8.495 mg, about 8.50 mg, about 8.505 mg, about 8.51 mg, about 8.515 mg, about 8.52 mg, about 8.525 mg, about 8.53 mg, about 8.535 mg, about 8.54 mg, about 8.545 mg, about 8.55 mg, about 8.555 mg, about 8.56 mg, about 8.565 mg, about 8.57 mg, about 8.575 mg, about 8.58 mg, about 8.585 mg, about 8.59 mg, about 8.595 mg, about 8.60 mg, about 8.605 mg, about 8.61 mg, about 8.615 mg, about 8.62 mg, about 8.625 mg, about 8.63 mg, about 8.635 mg, about 8.64 mg, about 8.645 mg, about 8.65 mg, about 8.655 mg, about 8.66 mg, about 8.665 mg, about 8.67 mg, about 8.675 mg, about 8.68 mg, about 8.685 mg, about 8.69 mg, about 8.695 mg, about 8.70 mg, about 8.705 mg, about 8.71 mg, about 8.715 mg, about 8.72 mg, about 8.725 mg, about 8.73 mg, about 8.735 mg, about 8.74 mg, about 8.745 mg, about 8.75 mg, about 8.755 mg, about 8.76 mg, about 8.765 mg, about 8.77 mg, about 8.775 mg, about 8.78 mg, about 8.785 mg, about 8.79 mg, about 8.795 mg, about 8.80 mg, about 8.805 mg, about 8.81 mg, about 8.815 mg, about 8.82 mg, about 8.825 mg, about 8.83 mg, about 8.835 mg, about 8.84 mg, about 8.845 mg, about 8.85 mg, about 8.855 mg, about 8.86 mg, about 8.865 mg, about 8.87 mg, about 8.875 mg, about 8.88 mg, about 8.885 mg, about 8.89 mg, about 8.895 mg, about 8.90 mg, about 8.905 mg, about 8.91 mg, about 8.915 mg, about 8.92 mg, about 8.925 mg, about 8.93 mg, about 8.935 mg, about 8.94 mg, about 8.945 mg, about 8.95 mg, about 8.955 mg, about 8.96 mg, about 8.965 mg, about 8.97 mg, about 8.975 mg, about 8.98 mg, about 8.985 mg, about 8.99 mg, or about 8.995 mg.

In some embodiments, the average dose of BCP is about 9.00 mg, about, about 9.005 mg, about 9.01 mg, about 9.015 mg, about 9.02 mg, about 9.025 mg, about 9.03 mg, about 9.035 mg, about 9.04 mg, about 9.045 mg, about 9.05 mg, about 9.055 mg, about 9.06 mg, about 9.065 mg, about 9.07 mg, about 9.075 mg, about 9.08 mg, about 9.085 mg, about 9.09 mg, about 9.095 mg, about 9.10 mg, about 9.105 mg, about 9.11 mg, about 9.115 mg, about 9.12 mg, about 9.125 mg, about 9.13 mg, about 9.135 mg, about 9.14 mg, about 9.145 mg, about 9.15 mg, about 9.155 mg, about 9.16 mg, about 9.165 mg, about 9.17 mg, about 9.175 mg, about 9.18 mg, about 9.185 mg, about 9.19 mg, about 9.195 mg, about 9.20 mg, about 9.205 mg, about 9.21 mg, about 9.215 mg, about 9.22 mg, about 9.225 mg, about 9.23 mg, about 9.235 mg, about 9.24 mg, about 9.245 mg, about 9.25 mg, about 9.255 mg, about 9.26 mg, about 9.265 mg, about 9.27 mg, about 9.275 mg, about 9.28 mg, about 9.285 mg, about 9.29 mg, about 9.295 mg, about 9.30 mg, about 9.305 mg, about 9.31 mg, about 9.315 mg, about 9.32 mg, about 9.325 mg, about 9.33 mg, about 9.335 mg, about 9.34 mg, about 9.345 mg, about 9.35 mg, about 9.355 mg, about 9.36 mg, about 9.365 mg, about 9.37 mg, about 9.375 mg, about 9.38 mg, about 9.385 mg, about 9.39 mg, about 9.395 mg, about 9.40 mg, about 9.405 mg, about 9.41 mg, about 9.415 mg, about 9.42 mg, about 9.425 mg, about 9.43 mg, about 9.435 mg, about 9.44 mg, about 9.445 mg, about 9.45 mg, about 9.455 mg, about 9.46 mg, about 9.465 mg, about 9.47 mg, about 9.475 mg, about 9.48 mg, about 9.485 mg, about 9.49 mg, about 9.495 mg, about 9.50 mg, about 9.505 mg, about 9.51 mg, about 9.515 mg, about 9.52 mg, about 9.525 mg, about 9.53 mg, about 9.535 mg, about 9.54 mg, about 9.545 mg, about 9.55 mg, about 9.555 mg, about 9.56 mg, about 9.565 mg, about 9.57 mg, about 9.575 mg, about 9.58 mg, about 9.585 mg, about 9.59 mg, about 9.595 mg, about 9.60 mg, about 9.605 mg, about 9.61 mg, about 9.615 mg, about 9.62 mg, about 9.625 mg, about 9.63 mg, about 9.635 mg, about 9.64 mg, about 9.645 mg, about 9.65 mg, about 9.655 mg, about 9.66 mg, about 9.665 mg, about 9.67 mg, about 9.675 mg, about 9.68 mg, about 9.685 mg, about 9.69 mg, about 9.695 mg, about 9.70 mg, about 9.705 mg, about 9.71 mg, about 9.715 mg, about 9.72 mg, about 9.725 mg, about 9.73 mg, about 9.735 mg, about 9.74 mg, about 9.745 mg, about 9.75 mg, about 9.755 mg, about 9.76 mg, about 9.765 mg, about 9.77 mg, about 9.775 mg, about 9.78 mg, about 9.785 mg, about 9.79 mg, about 9.795 mg, about 9.80 mg, about 9.805 mg, about 9.81 mg, about 9.815 mg, about 9.82 mg, about 9.825 mg, about 9.83 mg, about 9.835 mg, about 9.84 mg, about 9.845 mg, about 9.85 mg, about 9.855 mg, about 9.86 mg, about 9.865 mg, about 9.87 mg, about 9.875 mg, about 9.88 mg, about 9.885 mg, about 9.89 mg, about 9.895 mg, about 9.90 mg, about 9.905 mg, about 9.91 mg, about 9.915 mg, about 9.92 mg, about 9.925 mg, about 9.93 mg, about 9.935 mg, about 9.94 mg, about 9.945 mg, about 9.95 mg, about 9.955 mg, about 9.96 mg, about 9.965 mg, about 9.97 mg, about 9.975 mg, about 9.98 mg, about 9.985 mg, about 9.99 mg, about 9.995 mg, or about 10 mg.

In some embodiments, the average dose of BCP is about 10.0 mg, about 11.0 mg, about 12.0 mg, about 13.0 mg, about 14.0 mg, about 15.0 mg, about 16.0 mg, about 17.0 mg, about 18.0 mg, about 19.0 mg, about 20.0 mg, about 21.0 mg, about 22.0 mg, about 23.0 mg, about 24.0 mg, about 25.0 mg, about 26.0 mg, about 27.0 mg, about 28.0 mg, about 29.0 mg, about 30.0 mg, about 31.0 mg, about 32.0 mg, about 33.0 mg, about 34.0 mg, about 34.0 mg, about 36.0 mg, about 37.0 mg, about 38.0 mg, about 39.0 mg, about 40.0 mg, about 41.0 mg, about 42.0 mg, about 43.0 mg, about 44.0 mg, about 45.0 mg, about 46.0 mg, about 47.0 mg, about 48.0 mg, about 49.0 mg, about 50.0 mg, about 51.0 mg, about 52.0 mg, about 53.0 mg, about 54.0 mg, about 55.0 mg, about 56.0 mg, about 57.0 mg, about 58.0 mg, about 59.0 mg, about 60.0 mg, about 61.0 mg, about 62.0 mg, about 63.0 mg, about 64.0 mg, about 65.0 mg, about 66.0 mg, about 67.0 mg, about 68.0 mg, about 69.0 mg, about 70.0 mg, about 71.0 mg, about 72.0 mg, about 73.0 mg, about 74.0 mg, about 75.0 mg, about 76.0 mg, about 77.0 mg, about 78.0 mg, about 79.0 mg, about 80.0 mg, about 81.0 mg, about 82.0 mg, about 83.0 mg, about 84.0 mg, about 85.0 mg, about 86.0 mg, about 87.0 mg, about 88.0 mg, about 89.0 mg, about 90.0 mg, about 91.0 mg, about 92.0 mg, about 93.0 mg, about 94.0 mg, about 95.0 mg, about 96.0 mg, about 97.0 mg, about 98.0 mg, about 99.0 mg, or about 100 mg.

In some embodiments of the disclosure, the methods of treatment can include where the average dose of BCP is administered to a subject in need thereof with a frequency from about once a week to about 100 times per day, for example once per week, 2 times per week, 3 times per week, 4 times per week, 5 times per week, 6 times per week, once a day, 2 times per day, 3 times per day, 4 times per day, 5 times per day, 6 times per day, 7 times per day, 8 times per day, 9 times per day, 10 times per day, 11 time per day, 12 times per day, 13 times per day, 14 times per day, 15 times per day, 16 times per day, 17 times per day, 18 times per day, 19 times per day, 20 times per day, 21 times per day, 22 times per day, 23 times per day, 24 times per day, 25 times per day, 26 times per day, 27 times per day, 28 times per day, 29 times per day, 30 times per day, 31 times per day, 32 times per day, 33 times per day, 34 times per day, 35 times per day, 36 times per day, 37 times per day, 38 times per day, 39 times per day, 40 times per day, 41 times per day, 42 times per day, 43 times per day, 44 times per day, 45 times per day, 46 times per day, 47 times per day, 48 times per day, 49 times per day, 50 times per day, 55 times per day, 60 times per day, 70 times per day, 80 times per day, 90 times per day, or 100 times per day.

In some embodiments, inhaled BCP can be administered at or near the same time as another agent is administered to a patient or user such that the low-dose inhaled BCP has a synergistic effect with the other agent. For example, the inhaled BCP can be administered at the same time or shortly after (e.g., 1 min, 5 min, 10 min, 15 min, 20 min, 30 min, etc.) a patient has taken a dose of another medicine (such as a medicine for pain) via another administration method or route (e.g., orally (liquid or capsule), injected, and/or the like).

BCP compositions of the disclosure are pure, essentially pure, or substantially pure. The BCP compositions can be of about 50% purity, about 55% purity, about 60% purity, about 65% purity, about 70% purity, about 75% purity, about 80% purity, about 85% purity, about 90% purity, about 95% purity, about 99% purity, or greater than about 99% purity.

BCP compositions of the disclosure can be free, essentially free, or substantially free from one or more other terpenes, such as free, essentially free, or substantially free from one or more of: terpinolene, alpha phelladrene, beta ocimene, carene, limonene, gamma terpinene, alpha pinene, alpha terpinene, beta pinene, fenchol, camphene, alpha terpineol, alpha humulene, linalool, caryphyllene oxide, and/or myrcene.

BCP compositions of the disclosure can be free, essentially free, or substantially free from one or more other cannabinoids, such as free, essentially free, or substantially free from one or more of delta-9-tetrahydrocannabinol (THC) and/or cannabidiol (CBD).

BCP compositions of the disclosure can be free, essentially free, or substantially free from one or more carriers. For example, carriers that are not present in the BCP compositions can include borneol, camphor, 1,8-Cineole, citral, geraniol, indomethacin, limonene, linalool, linalyl acetate, β-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone.

In some embodiments, the BCP composition is administered via a handheld electronic device (BCP device) that vaporizes the BCP for inhalation by a user. In some embodiments, the BCP composition and/or BCP device do not include or contain additional constituents, carriers, or additives. For example, the BCP composition can be pure, essentially pure, or substantially pure, for example, having about 50% purity, about 55% purity, about 60% purity, about 65% purity, about 70% purity, about 75% purity, about 80% purity, about 85% purity, about 90% purity, about 95% purity, about 99% purity, or having greater than about 99% purity.

In some embodiments, additional components are optionally added to the BCP composition to improve the taste and/or physical properties of the composition (such as stability, viscosity, appearance of smoke as it is exhaled, etc.). Such additional components include, but are not limited to, sweeteners, natural flavorants, artificial flavorants, colorants, antioxidants, preservatives, chelating agents, viscomodulators, tonicifiers, odorants, opacifiers, suspending agents, binders, thickeners, carriers and mixtures thereof, including, but not limited to, xanthum gum, carboxymethylcellulose, carboxyethylcellulose, hydroxypropylcellulose, methylcellulose, microcrystalline cellulose, starches, dextrins, maltodextrins, other polyols (including sugar alcohols, such as sorbitol, lactitol or mannitol), carbohydrates (e.g., lactose), propylene glycol alginate, gellan gum, guar, pectin, tragacanth gum, gum acacia, locust bean gum, gum arabic, mannitol, sucralose, silicon dioxide, stearic acid, hydroxypropyl methylcellulose, mono-, di- and triglycerides (acyl glycerols), ether and sugar acetates or other acid esters such as dimethyl acetate, ethyl acetate, isopropyl acetate, ethylhexyl acetate, butyl acetate, triethyl citrate, dimethyl butyrate and the like.

In some embodiments, the carrier comprises one or more medium chain length triglycerides (MCTs). MCTs are triglycerides whose fatty acids have an aliphatic tail of 6-12 carbon atoms. In certain embodiments, the MCT is one or more of caproic acid, caprylic acid, capric acid, lauric acid and mixtures thereof. Suitable sources of MCTs are known to those skilled in the art and include, for example, coconut oil and palm kernel oil.

In some embodiments, the carrier comprises one or more polyesterdiols. The polyesterdiol may be a linear two to ten units polymer (also referred to as (ester)2-10 glycol), that is derived from natural or non-natural sources such as vegetables, fruits, bacteria, yeast, algae, or manufactured by chemical processes.

For example, in some embodiments, the polyesterdiol is 1) a polypropylene glycol such as: dipropylene glycol; tripropylene glycol, including tetra-, penta-, hexa-, hepta-, octa-, nona- and decapropylene glycol and other derivatives thereof; 2) a polybutylene glycol such as: dibutylene glycol, tributylene glycol, including tetra-, penta-, hexa-, hepta-, octa-, nona- and decabutylene glycol, and other derivatives thereof; 3) also including 2-10 unit polymers of rare organic ester types such as pentylene, octylene, terpentylene, nonylene, linalylene, isoamylene, isobutylene, geranylene, bornylene, benzylene and allylene, caprylylene, such as, for example, polyisobutylene glycol such as diisobutylene glycol; and 4) triethylene glycol, including tetra-, penta-, hexa-, hepta-, octa-, nona- and decaethylene glycols and other derivatives thereof such as acid or sugar conjugates, and esters or ether or alcohol derivatives.

In some embodiments, the carrier is a linear polyesterdiol selected from the group consisting of: (ethylene)3-10 glycol; (propylene)2-10 glycol; (butylene)2-10 glycol; (pentylene)2-10 glycol; (octylene)2-10 glycol; (terpentylene)2-10 glycol; (nonylene)2-10 glycol; (linalylene)2-10 glycol; (isoamylene)2-10 glycol; (isobutylene)2-10 glycol; (geranylene)2-10 glycol; (bornylene)2-10 glycol; (benzylene)2-10 glycol; (allylene)2-10 glycol; and (caprylylene)2-10 glycol; acid or sugar conjugates thereof, and ester or ether or alcohol derivatives thereof.

In some embodiments, the carrier is selected from the group consisting of: triethylene glycol; tetraethyleneglycol, pentaethylenglycol, hexaethyleneglycol, heptaethyleneglycol, octaethyleneglycol, nonaethylenglycol; decaethylene glycol; dipropylene glycol; tripropylene glycol; tetrapropylene glycol, pentapropylene glycol, hexapropylene glycol, heptapropylene glycol, octapropylene glycol, nonapropylene glycol; decapropylene glycol; dibutylene glycol, tributylene glycol; tetrabutylene glycol, pentabutylene glycol, hexabutylene glycol, heptabutylene glycol, octabutylene glycol, nonabutylene glycol, decabutylene glycol and diisobutylene glycol.

In some embodiments, the carrier is selected from the group consisting of borneol, camphor, 1,8-Cineole, citral, geraniol, indomethacin, limonene, linalool, linalyl acetate, β-myrcene, myrcenol, I-menthol, menthone, neomenthol, nerol, nerolidol, a-pinene, peppermint oil, pulegone, phytol, terpineol, terpinen-4-ol, thymohydroquinone, thymol, and thymoquinone

In some embodiments, the BCP composition comprises one or more of propylene glycol, glycerine, vegetable glycerine, and/or water.

If desired, one or more flavorants may be added to the composition. Non-limiting examples include wintergreen, propolis, eucalyptus, tobacco, Turkish tobacco, menthol, peppermint, spearmint, green tea, cream, natural sweetener, banana, strawberry, blueberry, mango, raspberry, peach, pineapple, apple, orange, papaya, watermelon, grape, cherry, coffee, chocolate, vanilla, cola, piña colada, coconut, almond, cinnamon, white chocolate, rum, butterscotch, and caramel or the like.

In some embodiments, the BCP composition is free from, essentially free from, or substantially free from one or more of: a carrier, propylene glycol, glycerine, vegetable glycerine, water, and/or flavorants. In some embodiments, the BCP composition is free from, essentially free from, or substantially free from one or more other terpenes and one or more other cannabinoids. It is to be understood that the percentages and purities discussed herein can be in weight or volume, depending on the embodiment and/or implementation.

In some embodiments, components of a BCP device include a mouthpiece, a BCP reservoir (which can be a tank or cartridge), a heating element or other atomizer (e.g., piezoelectric atomizer, inkjet-type atomizer, etc.), and an actuator (such as a switch or pressure sensor) to activate the heating element/atomizer. Depending on the embodiment, the BCP reservoir can be configured to store between about 0.5 mg and 500 mg of a BCP composition/BCP liquid, for example, between about 1 mg and about 250 mg, about 2 mg and 150 mg, about 3 mg and about 100 mg, about 4 mg and about 75 mg, about 5 mg and about 50 mg, or between about 10 mg and about 30 mg.

Depending on the embodiment, the BCP reservoir can be configured to store about 0.1 ml, about 0.5 ml, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, about 50 ml, about 55 ml, about 60 ml, about 70 ml, about 80 ml, about 90 ml, about 100 ml, or more than 100 ml.

Some implementations of the BCP device can include a microprocessor and a battery or other power source. Some implementations include and/or define a BCP vapor chamber or BCP atomization chamber. In some embodiments, an atomizer can include a small heater (e.g., heat coil or other heat element), that vaporizes a liquid BCP composition stored in the BCP reservoir. Some embodiments include a one or more wicks or wicking material configured to draw liquid BCP composition onto or into the heater or atomizer. In some embodiments, when a user activates an actuator (i.e., activates a pressure sensor by inhaling, presses a switch), the atomizer is activated (e.g., the heater is powered) and the liquid BCP composition is atomized or vaporized.

Depending on the embodiment and/or implementation, the heater and/or liquid BCP composition can reach a temperature from about 75° C. to about 350° C., from about 90° C. to about 300° C., from about 100° C. to about 290° C., from about 150° C. to about 280° C., from about 200° C. to about 275° C., from about 225° C. to about 270° C., from about 240° C. to about 265° C., or from about 250° C. to about 260° C. Some embodiments can include devices and/or configurations such as disclosed in one or more of the following, including combinations thereof: U.S. Pat. App. Pub. No. 2017/0023235; U.S. Pat. App. Pub. No. 2015/0114411; U.S. Pat. App. Pub. No. 2016/0000149; U.S. Pat. App. Pub. No. 2016/0270451; U.S. Pat. App. Pub. No. 2016/0053988; U.S. Pat. App. Pub. No. 2015/0327595; U.S. Pat. App. Pub. No. 2014/0299125; U.S. Pat. App. Pub. No. 2014/0182608; U.S. Pat. App. Pub. No. 2014/0286630; WIPO Patent Application Publication WO/2016/156497; WIPO Patent Application Publication WO/2016/030369; U.S. Pat. Nos. 9,320,300; 9,095,175; 9,259,035; 9,326,549; 7,997,280; 9,459,021; 9,427,711; and/or U.S. Pat. No. 9,352,288; each of which is expressly incorporated by reference herein for all purposes.

In some embodiments, the atomizer of a BCP device of the disclosure is configured to vaporize an average dose per actuation such that an average dose can be inhaled by a single inhalation of a user.

Alpha Caryophyllene

As detailed in the present disclosure, the Inventors have found beneficial and unexpected effects from administration of inhaled ACP.

According to some embodiments of the disclosure, there is provided vaporizable ACP compositions for use in a human subject in need thereof.

According to an aspect of some embodiments of the disclosure, there is also provided a device configured to provide vaporized ACP compositions as a medicament, and in particular, a device configured to provide vaporized ACP as a treatment for a variety of conditions in a subject in need thereof.

In some embodiments, such an ACP composition is configured for administration to a human subject via inhalation. In some embodiments, such an ACP composition is configured for administration via aerosolization. In some embodiments, such an ACP composition is configured for administration via heat-based vaporization.

According to an aspect of some embodiments of the disclosure, there is also provided a method for treating a variety of conditions in a human subject in need thereof, the method comprising administering a aerosolized ACP composition to the subject via inhalation.

In some embodiments, a vaporizable ACP composition according to the disclosure is provided as or made as an inhalable dosage form.

In some embodiments of a method of the disclosure, the average dose of ACP administered to a human subject via inhalation of a vapor thereof is from about 0.005 mg/kg to about 0.350 mg/kg, such as, for example, from about 0.006 mg/kg to about 0.340 mg/kg, from about 0.007 mg/kg to about 0.335 mg/kg, from about 0.008 mg/kg to about 0.330 mg/kg, from about 0.009 mg/kg to about 0.325 mg/kg, from about 0.01 mg/kg to about 0.320 mg/kg, from about 0.011 mg/kg to about 0.315 mg/kg, from about 0.012 mg/kg to about 0.310 mg/kg, from about 0.013 mg/kg to about 0.305 mg/kg, from about 0.014 mg/kg to about 0.300 mg/kg, from about 0.015 mg/kg to about 0.295 mg/kg, from about 0.016 mg/kg to about 0.290 mg/kg, from about 0.017 mg/kg to about 0.285 mg/kg, from about 0.018 mg/kg to about 0.280 mg/kg, from about 0.019 mg/kg to about 0.275 mg/kg, from about 0.020 mg/kg to about 0.270 mg/kg, from about 0.021 mg/kg to about 0.265 mg/kg, from about 0.022 mg/kg to about 0.260 mg/kg, from about 0.023 mg/kg to about 0.255 mg/kg, from about 0.024 mg/kg to about 0.250 mg/kg, from about 0.025 mg/kg to about 0.245 mg/kg, from about 0.026 mg/kg to about 0.240 mg/kg, from about 0.027 mg/kg to about 0.235 mg/kg, from about 0.028 mg/kg to about 0.230 mg/kg, from about 0.029 mg/kg to about 0.225 mg/kg, from about 0.030 mg/kg to about 0.220 mg/kg, from about 0.031 mg/kg to about 0.215 mg/kg, from about 0.032 mg/kg to about 0.210 mg/kg, from about 0.033 mg/kg to about 0.205 mg/kg, from about 0.034 mg/kg to about 0.200 mg/kg, from about 0.035 mg/kg to about 0.195 mg/kg, from about 0.036 mg/kg to about 0.190 mg/kg, from about 0.037 mg/kg to about 0.185 mg/kg, from about 0.038 mg/kg to about 0.180 mg/kg, from about 0.039 mg/kg to about 0.175 mg/kg, from about 0.040 mg/kg to about 0.170 mg/kg, from about 0.045 mg/kg to about 0.165 mg/kg, from about 0.050 mg/kg to about 0.160 mg/kg, from about 0.055 mg/kg to about 0.155 mg/kg, from about 0.060 mg/kg to about 0.150 mg/kg, from about 0.065 mg/kg to about 0.145 mg/kg, from about 0.070 mg/kg to about 0.140 mg/kg, from about 0.075 mg/kg to about 0.135 mg/kg, from about 0.080 mg/kg to about 0.130 mg/kg, from about 0.085 mg/kg to about 0.125 mg/kg, from about 0.090 mg/kg to about 0.120 mg/kg, from about 0.095 mg/kg to about 0.115 mg/kg, or from about 0.100 mg/kg to about 0.110 mg/kg,

In some embodiments, the average dose of ACP is about 0.001 mg, about 0.005 mg, about 0.01 mg, about 0.015 mg, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.10 mg, about 0.105 mg, about 0.11 mg, about 0.115 mg, about 0.12 mg, about 0.125 mg, about 0.13 mg, about 0.135 mg, about 0.14 mg, about 0.145 mg, about 0.15 mg, about 0.155 mg, about 0.16 mg, about 0.165 mg, about 0.17 mg, about 0.175 mg, about 0.18 mg, about 0.185 mg, about 0.19 mg, about 0.195 mg, about 0.20 mg, about 0.205 mg, about 0.21 mg, about 0.215 mg, about 0.22 mg, about 0.225 mg, about 0.23 mg, about 0.235 mg, about 0.24 mg, about 0.245 mg, about 0.25 mg, about 0.255 mg, about 0.26 mg, about 0.265 mg, about 0.27 mg, about 0.275 mg, about 0.28 mg, about 0.285 mg, about 0.29 mg, about 0.295 mg, about 0.30 mg, about 0.305 mg, about 0.31 mg, about 0.315 mg, about 0.32 mg, about 0.325 mg, about 0.33 mg, about 0.335 mg, about 0.34 mg, about 0.345 mg, about 0.35 mg, about 0.355 mg, about 0.36 mg, about 0.365 mg, about 0.37 mg, about 0.375 mg, about 0.38 mg, about 0.385 mg, about 0.39 mg, about 0.395 mg, about 0.40 mg, about 0.405 mg, about 0.41 mg, about 0.415 mg, about 0.42 mg, about 0.425 mg, about 0.43 mg, about 0.435 mg, about 0.44 mg, about 0.445 mg, about 0.45 mg, about 0.455 mg, about 0.46 mg, about 0.465 mg, about 0.47 mg, about 0.475 mg, about 0.48 mg, about 0.485 mg, about 0.49 mg, about 0.495 mg, about 0.50 mg, about 0.505 mg, about 0.51 mg, about 0.515 mg, about 0.52 mg, about 0.525 mg, about 0.53 mg, about 0.535 mg, about 0.54 mg, about 0.545 mg, about 0.55 mg, about 0.555 mg, about 0.56 mg, about 0.565 mg, about 0.57 mg, about 0.575 mg, about 0.58 mg, about 0.585 mg, about 0.59 mg, about 0.595 mg, about 0.60 mg, about 0.605 mg, about 0.61 mg, about 0.615 mg, about 0.62 mg, about 0.625 mg, about 0.63 mg, about 0.635 mg, about 0.64 mg, about 0.645 mg, about 0.65 mg, about 0.655 mg, about 0.66 mg, about 0.665 mg, about 0.67 mg, about 0.675 mg, about 0.68 mg, about 0.685 mg, about 0.69 mg, about 0.695 mg, about 0.70 mg, about 0.705 mg, about 0.71 mg, about 0.715 mg, about 0.72 mg, about 0.725 mg, about 0.73 mg, about 0.735 mg, about 0.74 mg, about 0.745 mg, about 0.75 mg, about 0.755 mg, about 0.76 mg, about 0.765 mg, about 0.77 mg, about 0.775 mg, about 0.78 mg, about 0.785 mg, about 0.79 mg, about 0.795 mg, about 0.80 mg, about 0.805 mg, about 0.81 mg, about 0.815 mg, about 0.82 mg, about 0.825 mg, about 0.83 mg, about 0.835 mg, about 0.84 mg, about 0.845 mg, about 0.85 mg, about 0.855 mg, about 0.86 mg, about 0.865 mg, about 0.87 mg, about 0.875 mg, about 0.88 mg, about 0.885 mg, about 0.89 mg, about 0.895 mg, about 0.90 mg, about 0.905 mg, about 0.91 mg, about 0.915 mg, about 0.92 mg, about 0.925 mg, about 0.93 mg, about 0.935 mg, about 0.94 mg, about 0.945 mg, about 0.95 mg, about 0.955 mg, about 0.96 mg, about 0.965 mg, about 0.97 mg, about 0.975 mg, about 0.98 mg, about 0.985 mg, about 0.99 mg, or about 0.995 mg.

In some embodiments, the average dose of ACP is about 1.00 mg, about, about 1.005 mg, about 1.01 mg, about 1.015 mg, about 1.02 mg, about 1.025 mg, about 1.03 mg, about 1.035 mg, about 1.04 mg, about 1.045 mg, about 1.05 mg, about 1.055 mg, about 1.06 mg, about 1.065 mg, about 1.07 mg, about 1.075 mg, about 1.08 mg, about 1.085 mg, about 1.09 mg, about 1.095 mg, about 1.10 mg, about 1.105 mg, about 1.11 mg, about 1.115 mg, about 1.12 mg, about 1.125 mg, about 1.13 mg, about 1.135 mg, about 1.14 mg, about 1.145 mg, about 1.15 mg, about 1.155 mg, about 1.16 mg, about 1.165 mg, about 1.17 mg, about 1.175 mg, about 1.18 mg, about 1.185 mg, about 1.19 mg, about 1.195 mg, about 1.20 mg, about 1.205 mg, about 1.21 mg, about 1.215 mg, about 1.22 mg, about 1.225 mg, about 1.23 mg, about 1.235 mg, about 1.24 mg, about 1.245 mg, about 1.25 mg, about 1.255 mg, about 1.26 mg, about 1.265 mg, about 1.27 mg, about 1.275 mg, about 1.28 mg, about 1.285 mg, about 1.29 mg, about 1.295 mg, about 1.30 mg, about 1.305 mg, about 1.31 mg, about 1.315 mg, about 1.32 mg, about 1.325 mg, about 1.33 mg, about 1.335 mg, about 1.34 mg, about 1.345 mg, about 1.35 mg, about 1.355 mg, about 1.36 mg, about 1.365 mg, about 1.37 mg, about 1.375 mg, about 1.38 mg, about 1.385 mg, about 1.39 mg, about 1.395 mg, about 1.40 mg, about 1.405 mg, about 1.41 mg, about 1.415 mg, about 1.42 mg, about 1.425 mg, about 1.43 mg, about 1.435 mg, about 1.44 mg, about 1.445 mg, about 1.45 mg, about 1.455 mg, about 1.46 mg, about 1.465 mg, about 1.47 mg, about 1.475 mg, about 1.48 mg, about 1.485 mg, about 1.49 mg, about 1.495 mg, about 1.50 mg, about 1.505 mg, about 1.51 mg, about 1.515 mg, about 1.52 mg, about 1.525 mg, about 1.53 mg, about 1.535 mg, about 1.54 mg, about 1.545 mg, about 1.55 mg, about 1.555 mg, about 1.56 mg, about 1.565 mg, about 1.57 mg, about 1.575 mg, about 1.58 mg, about 1.585 mg, about 1.59 mg, about 1.595 mg, about 1.60 mg, about 1.605 mg, about 1.61 mg, about 1.615 mg, about 1.62 mg, about 1.625 mg, about 1.63 mg, about 1.635 mg, about 1.64 mg, about 1.645 mg, about 1.65 mg, about 1.655 mg, about 1.66 mg, about 1.665 mg, about 1.67 mg, about 1.675 mg, about 1.68 mg, about 1.685 mg, about 1.69 mg, about 1.695 mg, about 1.70 mg, about 1.705 mg, about 1.71 mg, about 1.715 mg, about 1.72 mg, about 1.725 mg, about 1.73 mg, about 1.735 mg, about 1.74 mg, about 1.745 mg, about 1.75 mg, about 1.755 mg, about 1.76 mg, about 1.765 mg, about 1.77 mg, about 1.775 mg, about 1.78 mg, about 1.785 mg, about 1.79 mg, about 1.795 mg, about 1.80 mg, about 1.805 mg, about 1.81 mg, about 1.815 mg, about 1.82 mg, about 1.825 mg, about 1.83 mg, about 1.835 mg, about 1.84 mg, about 1.845 mg, about 1.85 mg, about 1.855 mg, about 1.86 mg, about 1.865 mg, about 1.87 mg, about 1.875 mg, about 1.88 mg, about 1.885 mg, about 1.89 mg, about 1.895 mg, about 1.90 mg, about 1.905 mg, about 1.91 mg, about 1.915 mg, about 1.92 mg, about 1.925 mg, about 1.93 mg, about 1.935 mg, about 1.94 mg, about 1.945 mg, about 1.95 mg, about 1.955 mg, about 1.96 mg, about 1.965 mg, about 1.97 mg, about 1.975 mg, about 1.98 mg, about 1.985 mg, about 1.99 mg, or about 1.995 mg.

In some embodiments, the average dose of ACP is about 2.00 mg, about, about 2.005 mg, about 2.01 mg, about 2.015 mg, about 2.02 mg, about 2.025 mg, about 2.03 mg, about 2.035 mg, about 2.04 mg, about 2.045 mg, about 2.05 mg, about 2.055 mg, about 2.06 mg, about 2.065 mg, about 2.07 mg, about 2.075 mg, about 2.08 mg, about 2.085 mg, about 2.09 mg, about 2.095 mg, about 2.10 mg, about 2.105 mg, about 2.11 mg, about 2.115 mg, about 2.12 mg, about 2.125 mg, about 2.13 mg, about 2.135 mg, about 2.14 mg, about 2.145 mg, about 2.15 mg, about 2.155 mg, about 2.16 mg, about 2.165 mg, about 2.17 mg, about 2.175 mg, about 2.18 mg, about 2.185 mg, about 2.19 mg, about 2.195 mg, about 2.20 mg, about 2.205 mg, about 2.21 mg, about 2.215 mg, about 2.22 mg, about 2.225 mg, about 2.23 mg, about 2.235 mg, about 2.24 mg, about 2.245 mg, about 2.25 mg, about 2.255 mg, about 2.26 mg, about 2.265 mg, about 2.27 mg, about 2.275 mg, about 2.28 mg, about 2.285 mg, about 2.29 mg, about 2.295 mg, about 2.30 mg, about 2.305 mg, about 2.31 mg, about 2.315 mg, about 2.32 mg, about 2.325 mg, about 2.33 mg, about 2.335 mg, about 2.34 mg, about 2.345 mg, about 2.35 mg, about 2.355 mg, about 2.36 mg, about 2.365 mg, about 2.37 mg, about 2.375 mg, about 2.38 mg, about 2.385 mg, about 2.39 mg, about 2.395 mg, about 2.40 mg, about 2.405 mg, about 2.41 mg, about 2.415 mg, about 2.42 mg, about 2.425 mg, about 2.43 mg, about 2.435 mg, about 2.44 mg, about 2.445 mg, about 2.45 mg, about 2.455 mg, about 2.46 mg, about 2.465 mg, about 2.47 mg, about 2.475 mg, about 2.48 mg, about 2.485 mg, about 2.49 mg, about 2.495 mg, about 2.50 mg, about 2.505 mg, about 2.51 mg, about 2.515 mg, about 2.52 mg, about 2.525 mg, about 2.53 mg, about 2.535 mg, about 2.54 mg, about 2.545 mg, about 2.55 mg, about 2.555 mg, about 2.56 mg, about 2.565 mg, about 2.57 mg, about 2.575 mg, about 2.58 mg, about 2.585 mg, about 2.59 mg, about 2.595 mg, about 2.60 mg, about 2.605 mg, about 2.61 mg, about 2.615 mg, about 2.62 mg, about 2.625 mg, about 2.63 mg, about 2.635 mg, about 2.64 mg, about 2.645 mg, about 2.65 mg, about 2.655 mg, about 2.66 mg, about 2.665 mg, about 2.67 mg, about 2.675 mg, about 2.68 mg, about 2.685 mg, about 2.69 mg, about 2.695 mg, about 2.70 mg, about 2.705 mg, about 2.71 mg, about 2.715 mg, about 2.72 mg, about 2.725 mg, about 2.73 mg, about 2.735 mg, about 2.74 mg, about 2.745 mg, about 2.75 mg, about 2.755 mg, about 2.76 mg, about 2.765 mg, about 2.77 mg, about 2.775 mg, about 2.78 mg, about 2.785 mg, about 2.79 mg, about 2.795 mg, about 2.80 mg, about 2.805 mg, about 2.81 mg, about 2.815 mg, about 2.82 mg, about 2.825 mg, about 2.83 mg, about 2.835 mg, about 2.84 mg, about 2.845 mg, about 2.85 mg, about 2.855 mg, about 2.86 mg, about 2.865 mg, about 2.87 mg, about 2.875 mg, about 2.88 mg, about 2.885 mg, about 2.89 mg, about 2.895 mg, about 2.90 mg, about 2.905 mg, about 2.91 mg, about 2.915 mg, about 2.92 mg, about 2.925 mg, about 2.93 mg, about 2.935 mg, about 2.94 mg, about 2.945 mg, about 2.95 mg, about 2.955 mg, about 2.96 mg, about 2.965 mg, about 2.97 mg, about 2.975 mg, about 2.98 mg, about 2.985 mg, about 2.99 mg, or about 2.995 mg.

In some embodiments, the average dose of ACP is about 3.00 mg, about, about 3.005 mg, about 3.01 mg, about 3.015 mg, about 3.02 mg, about 3.025 mg, about 3.03 mg, about 3.035 mg, about 3.04 mg, about 3.045 mg, about 3.05 mg, about 3.055 mg, about 3.06 mg, about 3.065 mg, about 3.07 mg, about 3.075 mg, about 3.08 mg, about 3.085 mg, about 3.09 mg, about 3.095 mg, about 3.10 mg, about 3.105 mg, about 3.11 mg, about 3.115 mg, about 3.12 mg, about 3.125 mg, about 3.13 mg, about 3.135 mg, about 3.14 mg, about 3.145 mg, about 3.15 mg, about 3.155 mg, about 3.16 mg, about 3.165 mg, about 3.17 mg, about 3.175 mg, about 3.18 mg, about 3.185 mg, about 3.19 mg, about 3.195 mg, about 3.20 mg, about 3.205 mg, about 3.21 mg, about 3.215 mg, about 3.22 mg, about 3.225 mg, about 3.23 mg, about 3.235 mg, about 3.24 mg, about 3.245 mg, about 3.25 mg, about 3.255 mg, about 3.26 mg, about 3.265 mg, about 3.27 mg, about 3.275 mg, about 3.28 mg, about 3.285 mg, about 3.29 mg, about 3.295 mg, about 3.30 mg, about 3.305 mg, about 3.31 mg, about 3.315 mg, about 3.32 mg, about 3.325 mg, about 3.33 mg, about 3.335 mg, about 3.34 mg, about 3.345 mg, about 3.35 mg, about 3.355 mg, about 3.36 mg, about 3.365 mg, about 3.37 mg, about 3.375 mg, about 3.38 mg, about 3.385 mg, about 3.39 mg, about 3.395 mg, about 3.40 mg, about 3.405 mg, about 3.41 mg, about 3.415 mg, about 3.42 mg, about 3.425 mg, about 3.43 mg, about 3.435 mg, about 3.44 mg, about 3.445 mg, about 3.45 mg, about 3.455 mg, about 3.46 mg, about 3.465 mg, about 3.47 mg, about 3.475 mg, about 3.48 mg, about 3.485 mg, about 3.49 mg, about 3.495 mg, about 3.50 mg, about 3.505 mg, about 3.51 mg, about 3.515 mg, about 3.52 mg, about 3.525 mg, about 3.53 mg, about 3.535 mg, about 3.54 mg, about 3.545 mg, about 3.55 mg, about 3.555 mg, about 3.56 mg, about 3.565 mg, about 3.57 mg, about 3.575 mg, about 3.58 mg, about 3.585 mg, about 3.59 mg, about 3.595 mg, about 3.60 mg, about 3.605 mg, about 3.61 mg, about 3.615 mg, about 3.62 mg, about 3.625 mg, about 3.63 mg, about 3.635 mg, about 3.64 mg, about 3.645 mg, about 3.65 mg, about 3.655 mg, about 3.66 mg, about 3.665 mg, about 3.67 mg, about 3.675 mg, about 3.68 mg, about 3.685 mg, about 3.69 mg, about 3.695 mg, about 3.70 mg, about 3.705 mg, about 3.71 mg, about 3.715 mg, about 3.72 mg, about 3.725 mg, about 3.73 mg, about 3.735 mg, about 3.74 mg, about 3.745 mg, about 3.75 mg, about 3.755 mg, about 3.76 mg, about 3.765 mg, about 3.77 mg, about 3.775 mg, about 3.78 mg, about 3.785 mg, about 3.79 mg, about 3.795 mg, about 3.80 mg, about 3.805 mg, about 3.81 mg, about 3.815 mg, about 3.82 mg, about 3.825 mg, about 3.83 mg, about 3.835 mg, about 3.84 mg, about 3.845 mg, about 3.85 mg, about 3.855 mg, about 3.86 mg, about 3.865 mg, about 3.87 mg, about 3.875 mg, about 3.88 mg, about 3.885 mg, about 3.89 mg, about 3.895 mg, about 3.90 mg, about 3.905 mg, about 3.91 mg, about 3.915 mg, about 3.92 mg, about 3.925 mg, about 3.93 mg, about 3.935 mg, about 3.94 mg, about 3.945 mg, about 3.95 mg, about 3.955 mg, about 3.96 mg, about 3.965 mg, about 3.97 mg, about 3.975 mg, about 3.98 mg, about 3.985 mg, about 3.99 mg, or about 3.995 mg.

In some embodiments, the average dose of ACP is about 4.00 mg, about, about 4.005 mg, about 4.01 mg, about 4.015 mg, about 4.02 mg, about 4.025 mg, about 4.03 mg, about 4.035 mg, about 4.04 mg, about 4.045 mg, about 4.05 mg, about 4.055 mg, about 4.06 mg, about 4.065 mg, about 4.07 mg, about 4.075 mg, about 4.08 mg, about 4.085 mg, about 4.09 mg, about 4.095 mg, about 4.10 mg, about 4.105 mg, about 4.11 mg, about 4.115 mg, about 4.12 mg, about 4.125 mg, about 4.13 mg, about 4.135 mg, about 4.14 mg, about 4.145 mg, about 4.15 mg, about 4.155 mg, about 4.16 mg, about 4.165 mg, about 4.17 mg, about 4.175 mg, about 4.18 mg, about 4.185 mg, about 4.19 mg, about 4.195 mg, about 4.20 mg, about 4.205 mg, about 4.21 mg, about 4.215 mg, about 4.22 mg, about 4.225 mg, about 4.23 mg, about 4.235 mg, about 4.24 mg, about 4.245 mg, about 4.25 mg, about 4.255 mg, about 4.26 mg, about 4.265 mg, about 4.27 mg, about 4.275 mg, about 4.28 mg, about 4.285 mg, about 4.29 mg, about 4.295 mg, about 4.30 mg, about 4.305 mg, about 4.31 mg, about 4.315 mg, about 4.32 mg, about 4.325 mg, about 4.33 mg, about 4.335 mg, about 4.34 mg, about 4.345 mg, about 4.35 mg, about 4.355 mg, about 4.36 mg, about 4.365 mg, about 4.37 mg, about 4.375 mg, about 4.38 mg, about 4.385 mg, about 4.39 mg, about 4.395 mg, about 4.40 mg, about 4.405 mg, about 4.41 mg, about 4.415 mg, about 4.42 mg, about 4.425 mg, about 4.43 mg, about 4.435 mg, about 4.44 mg, about 4.445 mg, about 4.45 mg, about 4.455 mg, about 4.46 mg, about 4.465 mg, about 4.47 mg, about 4.475 mg, about 4.48 mg, about 4.485 mg, about 4.49 mg, about 4.495 mg, about 4.50 mg, about 4.505 mg, about 4.51 mg, about 4.515 mg, about 4.52 mg, about 4.525 mg, about 4.53 mg, about 4.535 mg, about 4.54 mg, about 4.545 mg, about 4.55 mg, about 4.555 mg, about 4.56 mg, about 4.565 mg, about 4.57 mg, about 4.575 mg, about 4.58 mg, about 4.585 mg, about 4.59 mg, about 4.595 mg, about 4.60 mg, about 4.605 mg, about 4.61 mg, about 4.615 mg, about 4.62 mg, about 4.625 mg, about 4.63 mg, about 4.635 mg, about 4.64 mg, about 4.645 mg, about 4.65 mg, about 4.655 mg, about 4.66 mg, about 4.665 mg, about 4.67 mg, about 4.675 mg, about 4.68 mg, about 4.685 mg, about 4.69 mg, about 4.695 mg, about 4.70 mg, about 4.705 mg, about 4.71 mg, about 4.715 mg, about 4.72 mg, about 4.725 mg, about 4.73 mg, about 4.735 mg, about 4.74 mg, about 4.745 mg, about 4.75 mg, about 4.755 mg, about 4.76 mg, about 4.765 mg, about 4.77 mg, about 4.775 mg, about 4.78 mg, about 4.785 mg, about 4.79 mg, about 4.795 mg, about 4.80 mg, about 4.805 mg, about 4.81 mg, about 4.815 mg, about 4.82 mg, about 4.825 mg, about 4.83 mg, about 4.835 mg, about 4.84 mg, about 4.845 mg, about 4.85 mg, about 4.855 mg, about 4.86 mg, about 4.865 mg, about 4.87 mg, about 4.875 mg, about 4.88 mg, about 4.885 mg, about 4.89 mg, about 4.895 mg, about 4.90 mg, about 4.905 mg, about 4.91 mg, about 4.915 mg, about 4.92 mg, about 4.925 mg, about 4.93 mg, about 4.935 mg, about 4.94 mg, about 4.945 mg, about 4.95 mg, about 4.955 mg, about 4.96 mg, about 4.965 mg, about 4.97 mg, about 4.975 mg, about 4.98 mg, about 4.985 mg, about 4.99 mg, or about 4.995 mg.

In some embodiments, the average dose of ACP is about 5.00 mg, about, about 5.005 mg, about 5.01 mg, about 5.015 mg, about 5.02 mg, about 5.025 mg, about 5.03 mg, about 5.035 mg, about 5.04 mg, about 5.045 mg, about 5.05 mg, about 5.055 mg, about 5.06 mg, about 5.065 mg, about 5.07 mg, about 5.075 mg, about 5.08 mg, about 5.085 mg, about 5.09 mg, about 5.095 mg, about 5.10 mg, about 5.105 mg, about 5.11 mg, about 5.115 mg, about 5.12 mg, about 5.125 mg, about 5.13 mg, about 5.135 mg, about 5.14 mg, about 5.145 mg, about 5.15 mg, about 5.155 mg, about 5.16 mg, about 5.165 mg, about 5.17 mg, about 5.175 mg, about 5.18 mg, about 5.185 mg, about 5.19 mg, about 5.195 mg, about 5.20 mg, about 5.205 mg, about 5.21 mg, about 5.215 mg, about 5.22 mg, about 5.225 mg, about 5.23 mg, about 5.235 mg, about 5.24 mg, about 5.245 mg, about 5.25 mg, about 5.255 mg, about 5.26 mg, about 5.265 mg, about 5.27 mg, about 5.275 mg, about 5.28 mg, about 5.285 mg, about 5.29 mg, about 5.295 mg, about 5.30 mg, about 5.305 mg, about 5.31 mg, about 5.315 mg, about 5.32 mg, about 5.325 mg, about 5.33 mg, about 5.335 mg, about 5.34 mg, about 5.345 mg, about 5.35 mg, about 5.355 mg, about 5.36 mg, about 5.365 mg, about 5.37 mg, about 5.375 mg, about 5.38 mg, about 5.385 mg, about 5.39 mg, about 5.395 mg, about 5.40 mg, about 5.405 mg, about 5.41 mg, about 5.415 mg, about 5.42 mg, about 5.425 mg, about 5.43 mg, about 5.435 mg, about 5.44 mg, about 5.445 mg, about 5.45 mg, about 5.455 mg, about 5.46 mg, about 5.465 mg, about 5.47 mg, about 5.475 mg, about 5.48 mg, about 5.485 mg, about 5.49 mg, about 5.495 mg, about 5.50 mg, about 5.505 mg, about 5.51 mg, about 5.515 mg, about 5.52 mg, about 5.525 mg, about 5.53 mg, about 5.535 mg, about 5.54 mg, about 5.545 mg, about 5.55 mg, about 5.555 mg, about 5.56 mg, about 5.565 mg, about 5.57 mg, about 5.575 mg, about 5.58 mg, about 5.585 mg, about 5.59 mg, about 5.595 mg, about 5.60 mg, about 5.605 mg, about 5.61 mg, about 5.615 mg, about 5.62 mg, about 5.625 mg, about 5.63 mg, about 5.635 mg, about 5.64 mg, about 5.645 mg, about 5.65 mg, about 5.655 mg, about 5.66 mg, about 5.665 mg, about 5.67 mg, about 5.675 mg, about 5.68 mg, about 5.685 mg, about 5.69 mg, about 5.695 mg, about 5.70 mg, about 5.705 mg, about 5.71 mg, about 5.715 mg, about 5.72 mg, about 5.725 mg, about 5.73 mg, about 5.735 mg, about 5.74 mg, about 5.745 mg, about 5.75 mg, about 5.755 mg, about 5.76 mg, about 5.765 mg, about 5.77 mg, about 5.775 mg, about 5.78 mg, about 5.785 mg, about 5.79 mg, about 5.795 mg, about 5.80 mg, about 5.805 mg, about 5.81 mg, about 5.815 mg, about 5.82 mg, about 5.825 mg, about 5.83 mg, about 5.835 mg, about 5.84 mg, about 5.845 mg, about 5.85 mg, about 5.855 mg, about 5.86 mg, about 5.865 mg, about 5.87 mg, about 5.875 mg, about 5.88 mg, about 5.885 mg, about 5.89 mg, about 5.895 mg, about 5.90 mg, about 5.905 mg, about 5.91 mg, about 5.915 mg, about 5.92 mg, about 5.925 mg, about 5.93 mg, about 5.935 mg, about 5.94 mg, about 5.945 mg, about 5.95 mg, about 5.955 mg, about 5.96 mg, about 5.965 mg, about 5.97 mg, about 5.975 mg, about 5.98 mg, about 5.985 mg, about 5.99 mg, or about 5.995 mg.

In some embodiments, the average dose of ACP is about 6.00 mg, about, about 6.005 mg, about 6.01 mg, about 6.015 mg, about 6.02 mg, about 6.025 mg, about 6.03 mg, about 6.035 mg, about 6.04 mg, about 6.045 mg, about 6.05 mg, about 6.055 mg, about 6.06 mg, about 6.065 mg, about 6.07 mg, about 6.075 mg, about 6.08 mg, about 6.085 mg, about 6.09 mg, about 6.095 mg, about 6.10 mg, about 6.105 mg, about 6.11 mg, about 6.115 mg, about 6.12 mg, about 6.125 mg, about 6.13 mg, about 6.135 mg, about 6.14 mg, about 6.145 mg, about 6.15 mg, about 6.155 mg, about 6.16 mg, about 6.165 mg, about 6.17 mg, about 6.175 mg, about 6.18 mg, about 6.185 mg, about 6.19 mg, about 6.195 mg, about 6.20 mg, about 6.205 mg, about 6.21 mg, about 6.215 mg, about 6.22 mg, about 6.225 mg, about 6.23 mg, about 6.235 mg, about 6.24 mg, about 6.245 mg, about 6.25 mg, about 6.255 mg, about 6.26 mg, about 6.265 mg, about 6.27 mg, about 6.275 mg, about 6.28 mg, about 6.285 mg, about 6.29 mg, about 6.295 mg, about 6.30 mg, about 6.305 mg, about 6.31 mg, about 6.315 mg, about 6.32 mg, about 6.325 mg, about 6.33 mg, about 6.335 mg, about 6.34 mg, about 6.345 mg, about 6.35 mg, about 6.355 mg, about 6.36 mg, about 6.365 mg, about 6.37 mg, about 6.375 mg, about 6.38 mg, about 6.385 mg, about 6.39 mg, about 6.395 mg, about 6.40 mg, about 6.405 mg, about 6.41 mg, about 6.415 mg, about 6.42 mg, about 6.425 mg, about 6.43 mg, about 6.435 mg, about 6.44 mg, about 6.445 mg, about 6.45 mg, about 6.455 mg, about 6.46 mg, about 6.465 mg, about 6.47 mg, about 6.475 mg, about 6.48 mg, about 6.485 mg, about 6.49 mg, about 6.495 mg, about 6.50 mg, about 6.505 mg, about 6.51 mg, about 6.515 mg, about 6.52 mg, about 6.525 mg, about 6.53 mg, about 6.535 mg, about 6.54 mg, about 6.545 mg, about 6.55 mg, about 6.555 mg, about 6.56 mg, about 6.565 mg, about 6.57 mg, about 6.575 mg, about 6.58 mg, about 6.585 mg, about 6.59 mg, about 6.595 mg, about 6.60 mg, about 6.605 mg, about 6.61 mg, about 6.615 mg, about 6.62 mg, about 6.625 mg, about 6.63 mg, about 6.635 mg, about 6.64 mg, about 6.645 mg, about 6.65 mg, about 6.655 mg, about 6.66 mg, about 6.665 mg, about 6.67 mg, about 6.675 mg, about 6.68 mg, about 6.685 mg, about 6.69 mg, about 6.695 mg, about 6.70 mg, about 6.705 mg, about 6.71 mg, about 6.715 mg, about 6.72 mg, about 6.725 mg, about 6.73 mg, about 6.735 mg, about 6.74 mg, about 6.745 mg, about 6.75 mg, about 6.755 mg, about 6.76 mg, about 6.765 mg, about 6.77 mg, about 6.775 mg, about 6.78 mg, about 6.785 mg, about 6.79 mg, about 6.795 mg, about 6.80 mg, about 6.805 mg, about 6.81 mg, about 6.815 mg, about 6.82 mg, about 6.825 mg, about 6.83 mg, about 6.835 mg, about 6.84 mg, about 6.845 mg, about 6.85 mg, about 6.855 mg, about 6.86 mg, about 6.865 mg, about 6.87 mg, about 6.875 mg, about 6.88 mg, about 6.885 mg, about 6.89 mg, about 6.895 mg, about 6.90 mg, about 6.905 mg, about 6.91 mg, about 6.915 mg, about 6.92 mg, about 6.925 mg, about 6.93 mg, about 6.935 mg, about 6.94 mg, about 6.945 mg, about 6.95 mg, about 6.955 mg, about 6.96 mg, about 6.965 mg, about 6.97 mg, about 6.975 mg, about 6.98 mg, about 6.985 mg, about 6.99 mg, or about 6.995 mg.

In some embodiments, the average dose of ACP is about 7.00 mg, about, about 7.005 mg, about 7.01 mg, about 7.015 mg, about 7.02 mg, about 7.025 mg, about 7.03 mg, about 7.035 mg, about 7.04 mg, about 7.045 mg, about 7.05 mg, about 7.055 mg, about 7.06 mg, about 7.065 mg, about 7.07 mg, about 7.075 mg, about 7.08 mg, about 7.085 mg, about 7.09 mg, about 7.095 mg, about 7.10 mg, about 7.105 mg, about 7.11 mg, about 7.115 mg, about 7.12 mg, about 7.125 mg, about 7.13 mg, about 7.135 mg, about 7.14 mg, about 7.145 mg, about 7.15 mg, about 7.155 mg, about 7.16 mg, about 7.165 mg, about 7.17 mg, about 7.175 mg, about 7.18 mg, about 7.185 mg, about 7.19 mg, about 7.195 mg, about 7.20 mg, about 7.205 mg, about 7.21 mg, about 7.215 mg, about 7.22 mg, about 7.225 mg, about 7.23 mg, about 7.235 mg, about 7.24 mg, about 7.245 mg, about 7.25 mg, about 7.255 mg, about 7.26 mg, about 7.265 mg, about 7.27 mg, about 7.275 mg, about 7.28 mg, about 7.285 mg, about 7.29 mg, about 7.295 mg, about 7.30 mg, about 7.305 mg, about 7.31 mg, about 7.315 mg, about 7.32 mg, about 7.325 mg, about 7.33 mg, about 7.335 mg, about 7.34 mg, about 7.345 mg, about 7.35 mg, about 7.355 mg, about 7.36 mg, about 7.365 mg, about 7.37 mg, about 7.375 mg, about 7.38 mg, about 7.385 mg, about 7.39 mg, about 7.395 mg, about 7.40 mg, about 7.405 mg, about 7.41 mg, about 7.415 mg, about 7.42 mg, about 7.425 mg, about 7.43 mg, about 7.435 mg, about 7.44 mg, about 7.445 mg, about 7.45 mg, about 7.455 mg, about 7.46 mg, about 7.465 mg, about 7.47 mg, about 7.475 mg, about 7.48 mg, about 7.485 mg, about 7.49 mg, about 7.495 mg, about 7.50 mg, about 7.505 mg, about 7.51 mg, about 7.515 mg, about 7.52 mg, about 7.525 mg, about 7.53 mg, about 7.535 mg, about 7.54 mg, about 7.545 mg, about 7.55 mg, about 7.555 mg, about 7.56 mg, about 7.565 mg, about 7.57 mg, about 7.575 mg, about 7.58 mg, about 7.585 mg, about 7.59 mg, about 7.595 mg, about 7.60 mg, about 7.605 mg, about 7.61 mg, about 7.615 mg, about 7.62 mg, about 7.625 mg, about 7.63 mg, about 7.635 mg, about 7.64 mg, about 7.645 mg, about 7.65 mg, about 7.655 mg, about 7.66 mg, about 7.665 mg, about 7.67 mg, about 7.675 mg, about 7.68 mg, about 7.685 mg, about 7.69 mg, about 7.695 mg, about 7.70 mg, about 7.705 mg, about 7.71 mg, about 7.715 mg, about 7.72 mg, about 7.725 mg, about 7.73 mg, about 7.735 mg, about 7.74 mg, about 7.745 mg, about 7.75 mg, about 7.755 mg, about 7.76 mg, about 7.765 mg, about 7.77 mg, about 7.775 mg, about 7.78 mg, about 7.785 mg, about 7.79 mg, about 7.795 mg, about 7.80 mg, about 7.805 mg, about 7.81 mg, about 7.815 mg, about 7.82 mg, about 7.825 mg, about 7.83 mg, about 7.835 mg, about 7.84 mg, about 7.845 mg, about 7.85 mg, about 7.855 mg, about 7.86 mg, about 7.865 mg, about 7.87 mg, about 7.875 mg, about 7.88 mg, about 7.885 mg, about 7.89 mg, about 7.895 mg, about 7.90 mg, about 7.905 mg, about 7.91 mg, about 7.915 mg, about 7.92 mg, about 7.925 mg, about 7.93 mg, about 7.935 mg, about 7.94 mg, about 7.945 mg, about 7.95 mg, about 7.955 mg, about 7.96 mg, about 7.965 mg, about 7.97 mg, about 7.975 mg, about 7.98 mg, about 7.985 mg, about 7.99 mg, or about 7.995 mg.

In some embodiments, the average dose of ACP is about 8.00 mg, about, about 8.005 mg, about 8.01 mg, about 8.015 mg, about 8.02 mg, about 8.025 mg, about 8.03 mg, about 8.035 mg, about 8.04 mg, about 8.045 mg, about 8.05 mg, about 8.055 mg, about 8.06 mg, about 8.065 mg, about 8.07 mg, about 8.075 mg, about 8.08 mg, about 8.085 mg, about 8.09 mg, about 8.095 mg, about 8.10 mg, about 8.105 mg, about 8.11 mg, about 8.115 mg, about 8.12 mg, about 8.125 mg, about 8.13 mg, about 8.135 mg, about 8.14 mg, about 8.145 mg, about 8.15 mg, about 8.155 mg, about 8.16 mg, about 8.165 mg, about 8.17 mg, about 8.175 mg, about 8.18 mg, about 8.185 mg, about 8.19 mg, about 8.195 mg, about 8.20 mg, about 8.205 mg, about 8.21 mg, about 8.215 mg, about 8.22 mg, about 8.225 mg, about 8.23 mg, about 8.235 mg, about 8.24 mg, about 8.245 mg, about 8.25 mg, about 8.255 mg, about 8.26 mg, about 8.265 mg, about 8.27 mg, about 8.275 mg, about 8.28 mg, about 8.285 mg, about 8.29 mg, about 8.295 mg, about 8.30 mg, about 8.305 mg, about 8.31 mg, about 8.315 mg, about 8.32 mg, about 8.325 mg, about 8.33 mg, about 8.335 mg, about 8.34 mg, about 8.345 mg, about 8.35 mg, about 8.355 mg, about 8.36 mg, about 8.365 mg, about 8.37 mg, about 8.375 mg, about 8.38 mg, about 8.385 mg, about 8.39 mg, about 8.395 mg, about 8.40 mg, about 8.405 mg, about 8.41 mg, about 8.415 mg, about 8.42 mg, about 8.425 mg, about 8.43 mg, about 8.435 mg, about 8.44 mg, about 8.445 mg, about 8.45 mg, about 8.455 mg, about 8.46 mg, about 8.465 mg, about 8.47 mg, about 8.475 mg, about 8.48 mg, about 8.485 mg, about 8.49 mg, about 8.495 mg, about 8.50 mg, about 8.505 mg, about 8.51 mg, about 8.515 mg, about 8.52 mg, about 8.525 mg, about 8.53 mg, about 8.535 mg, about 8.54 mg, about 8.545 mg, about 8.55 mg, about 8.555 mg, about 8.56 mg, about 8.565 mg, about 8.57 mg, about 8.575 mg, about 8.58 mg, about 8.585 mg, about 8.59 mg, about 8.595 mg, about 8.60 mg, about 8.605 mg, about 8.61 mg, about 8.615 mg, about 8.62 mg, about 8.625 mg, about 8.63 mg, about 8.635 mg, about 8.64 mg, about 8.645 mg, about 8.65 mg, about 8.655 mg, about 8.66 mg, about 8.665 mg, about 8.67 mg, about 8.675 mg, about 8.68 mg, about 8.685 mg, about 8.69 mg, about 8.695 mg, about 8.70 mg, about 8.705 mg, about 8.71 mg, about 8.715 mg, about 8.72 mg, about 8.725 mg, about 8.73 mg, about 8.735 mg, about 8.74 mg, about 8.745 mg, about 8.75 mg, about 8.755 mg, about 8.76 mg, about 8.765 mg, about 8.77 mg, about 8.775 mg, about 8.78 mg, about 8.785 mg, about 8.79 mg, about 8.795 mg, about 8.80 mg, about 8.805 mg, about 8.81 mg, about 8.815 mg, about 8.82 mg, about 8.825 mg, about 8.83 mg, about 8.835 mg, about 8.84 mg, about 8.845 mg, about 8.85 mg, about 8.855 mg, about 8.86 mg, about 8.865 mg, about 8.87 mg, about 8.875 mg, about 8.88 mg, about 8.885 mg, about 8.89 mg, about 8.895 mg, about 8.90 mg, about 8.905 mg, about 8.91 mg, about 8.915 mg, about 8.92 mg, about 8.925 mg, about 8.93 mg, about 8.935 mg, about 8.94 mg, about 8.945 mg, about 8.95 mg, about 8.955 mg, about 8.96 mg, about 8.965 mg, about 8.97 mg, about 8.975 mg, about 8.98 mg, about 8.985 mg, about 8.99 mg, or about 8.995 mg.

In some embodiments, the average dose of ACP is about 9.00 mg, about, about 9.005 mg, about 9.01 mg, about 9.015 mg, about 9.02 mg, about 9.025 mg, about 9.03 mg, about 9.035 mg, about 9.04 mg, about 9.045 mg, about 9.05 mg, about 9.055 mg, about 9.06 mg, about 9.065 mg, about 9.07 mg, about 9.075 mg, about 9.08 mg, about 9.085 mg, about 9.09 mg, about 9.095 mg, about 9.10 mg, about 9.105 mg, about 9.11 mg, about 9.115 mg, about 9.12 mg, about 9.125 mg, about 9.13 mg, about 9.135 mg, about 9.14 mg, about 9.145 mg, about 9.15 mg, about 9.155 mg, about 9.16 mg, about 9.165 mg, about 9.17 mg, about 9.175 mg, about 9.18 mg, about 9.185 mg, about 9.19 mg, about 9.195 mg, about 9.20 mg, about 9.205 mg, about 9.21 mg, about 9.215 mg, about 9.22 mg, about 9.225 mg, about 9.23 mg, about 9.235 mg, about 9.24 mg, about 9.245 mg, about 9.25 mg, about 9.255 mg, about 9.26 mg, about 9.265 mg, about 9.27 mg, about 9.275 mg, about 9.28 mg, about 9.285 mg, about 9.29 mg, about 9.295 mg, about 9.30 mg, about 9.305 mg, about 9.31 mg, about 9.315 mg, about 9.32 mg, about 9.325 mg, about 9.33 mg, about 9.335 mg, about 9.34 mg, about 9.345 mg, about 9.35 mg, about 9.355 mg, about 9.36 mg, about 9.365 mg, about 9.37 mg, about 9.375 mg, about 9.38 mg, about 9.385 mg, about 9.39 mg, about 9.395 mg, about 9.40 mg, about 9.405 mg, about 9.41 mg, about 9.415 mg, about 9.42 mg, about 9.425 mg, about 9.43 mg, about 9.435 mg, about 9.44 mg, about 9.445 mg, about 9.45 mg, about 9.455 mg, about 9.46 mg, about 9.465 mg, about 9.47 mg, about 9.475 mg, about 9.48 mg, about 9.485 mg, about 9.49 mg, about 9.495 mg, about 9.50 mg, about 9.505 mg, about 9.51 mg, about 9.515 mg, about 9.52 mg, about 9.525 mg, about 9.53 mg, about 9.535 mg, about 9.54 mg, about 9.545 mg, about 9.55 mg, about 9.555 mg, about 9.56 mg, about 9.565 mg, about 9.57 mg, about 9.575 mg, about 9.58 mg, about 9.585 mg, about 9.59 mg, about 9.595 mg, about 9.60 mg, about 9.605 mg, about 9.61 mg, about 9.615 mg, about 9.62 mg, about 9.625 mg, about 9.63 mg, about 9.635 mg, about 9.64 mg, about 9.645 mg, about 9.65 mg, about 9.655 mg, about 9.66 mg, about 9.665 mg, about 9.67 mg, about 9.675 mg, about 9.68 mg, about 9.685 mg, about 9.69 mg, about 9.695 mg, about 9.70 mg, about 9.705 mg, about 9.71 mg, about 9.715 mg, about 9.72 mg, about 9.725 mg, about 9.73 mg, about 9.735 mg, about 9.74 mg, about 9.745 mg, about 9.75 mg, about 9.755 mg, about 9.76 mg, about 9.765 mg, about 9.77 mg, about 9.775 mg, about 9.78 mg, about 9.785 mg, about 9.79 mg, about 9.795 mg, about 9.80 mg, about 9.805 mg, about 9.81 mg, about 9.815 mg, about 9.82 mg, about 9.825 mg, about 9.83 mg, about 9.835 mg, about 9.84 mg, about 9.845 mg, about 9.85 mg, about 9.855 mg, about 9.86 mg, about 9.865 mg, about 9.87 mg, about 9.875 mg, about 9.88 mg, about 9.885 mg, about 9.89 mg, about 9.895 mg, about 9.90 mg, about 9.905 mg, about 9.91 mg, about 9.915 mg, about 9.92 mg, about 9.925 mg, about 9.93 mg, about 9.935 mg, about 9.94 mg, about 9.945 mg, about 9.95 mg, about 9.955 mg, about 9.96 mg, about 9.965 mg, about 9.97 mg, about 9.975 mg, about 9.98 mg, about 9.985 mg, about 9.99 mg, about 9.995 mg, or about 10 mg.

In some embodiments, the average dose of ACP is about 10.0 mg, about 11.0 mg, about 12.0 mg, about 13.0 mg, about 14.0 mg, about 15.0 mg, about 16.0 mg, about 17.0 mg, about 18.0 mg, about 19.0 mg, about 20.0 mg, about 21.0 mg, about 22.0 mg, about 23.0 mg, about 24.0 mg, about 25.0 mg, about 26.0 mg, about 27.0 mg, about 28.0 mg, about 29.0 mg, about 30.0 mg, about 31.0 mg, about 32.0 mg, about 33.0 mg, about 34.0 mg, about 34.0 mg, about 36.0 mg, about 37.0 mg, about 38.0 mg, about 39.0 mg, about 40.0 mg, about 41.0 mg, about 42.0 mg, about 43.0 mg, about 44.0 mg, about 45.0 mg, about 46.0 mg, about 47.0 mg, about 48.0 mg, about 49.0 mg, about 50.0 mg, about 51.0 mg, about 52.0 mg, about 53.0 mg, about 54.0 mg, about 55.0 mg, about 56.0 mg, about 57.0 mg, about 58.0 mg, about 59.0 mg, about 60.0 mg, about 61.0 mg, about 62.0 mg, about 63.0 mg, about 64.0 mg, about 65.0 mg, about 66.0 mg, about 67.0 mg, about 68.0 mg, about 69.0 mg, about 70.0 mg, about 71.0 mg, about 72.0 mg, about 73.0 mg, about 74.0 mg, about 75.0 mg, about 76.0 mg, about 77.0 mg, about 78.0 mg, about 79.0 mg, about 80.0 mg, about 81.0 mg, about 82.0 mg, about 83.0 mg, about 84.0 mg, about 85.0 mg, about 86.0 mg, about 87.0 mg, about 88.0 mg, about 89.0 mg, about 90.0 mg, about 91.0 mg, about 92.0 mg, about 93.0 mg, about 94.0 mg, about 95.0 mg, about 96.0 mg, about 97.0 mg, about 98.0 mg, about 99.0 mg, or about 100 mg.

In some embodiments of the disclosure, the methods of treatment can include where the average dose of ACP is administered to a subject in need thereof with a frequency from about once a week to about 100 times per day, for example once per week, 2 times per week, 3 times per week, 4 times per week, 5 times per week, 6 times per week, once a day, 2 times per day, 3 times per day, 4 times per day, 5 times per day, 6 times per day, 7 times per day, 8 times per day, 9 times per day, 10 times per day, 11 time per day, 12 times per day, 13 times per day, 14 times per day, 15 times per day, 16 times per day, 17 times per day, 18 times per day, 19 times per day, 20 times per day, 21 times per day, 22 times per day, 23 times per day, 24 times per day, 25 times per day, 26 times per day, 27 times per day, 28 times per day, 29 times per day, 30 times per day, 31 times per day, 32 times per day, 33 times per day, 34 times per day, 35 times per day, 36 times per day, 37 times per day, 38 times per day, 39 times per day, 40 times per day, 41 times per day, 42 times per day, 43 times per day, 44 times per day, 45 times per day, 46 times per day, 47 times per day, 48 times per day, 49 times per day, 50 times per day, 55 times per day, 60 times per day, 70 times per day, 80 times per day, 90 times per day, or 100 times per day.

In some embodiments, inhaled ACP can be administered at or near the same time as another agent is administered to a patient or user such that the low-dose inhaled ACP has a synergistic effect with the other agent. For example, the inhaled ACP can be administered at the same time or shortly after (e.g., 1 min, 5 min, 10 min, 15 min, 20 min, 30 min, etc.) a patient has taken a dose of another medicine (such as a medicine for pain) via another administration method or route (e.g., orally (liquid or capsule), injected, and/or the like).

ACP compositions of the disclosure are pure, essentially pure, or substantially pure. The ACP compositions can be of about 50% purity, about 55% purity, about 60% purity, about 65% purity, about 70% purity, about 75% purity, about 80% purity, about 85% purity, about 90% purity, about 95% purity, about 99% purity, or greater than about 99% purity.

In some embodiments, additional components are optionally added to the ACP composition to improve the taste and/or physical properties of the composition (such as stability, viscosity, appearance of smoke as it is exhaled, etc.). Such additional components include, but are not limited to, sweeteners, natural flavorants, artificial flavorants, colorants, antioxidants, preservatives, chelating agents, viscomodulators, tonicifiers, odorants, opacifiers, suspending agents, binders, thickeners, carriers and mixtures thereof, including, but not limited to, xanthum gum, carboxymethylcellulose, carboxyethylcellulose, hydroxypropylcellulose, methylcellulose, microcrystalline cellulose, starches, dextrins, maltodextrins, other polyols (including sugar alcohols, such as sorbitol, lactitol or mannitol), carbohydrates (e.g., lactose), propylene glycol alginate, gellan gum, guar, pectin, tragacanth gum, gum acacia, locust bean gum, gum arabic, mannitol, sucralose, silicon dioxide, stearic acid, hydroxypropyl methylcellulose, mono-, di- and triglycerides (acyl glycerols), ether and sugar acetates or other acid esters such as dimethyl acetate, ethyl acetate, isopropyl acetate, ethylhexyl acetate, butyl acetate, triethyl citrate, dimethyl butyrate and the like.

In some embodiments, the carrier comprises one or more medium chain length triglycerides (MCTs). MCTs are triglycerides whose fatty acids have an aliphatic tail of 6-12 carbon atoms. In certain embodiments, the MCT is one or more of caproic acid, caprylic acid, capric acid, lauric acid and mixtures thereof. Suitable sources of MCTs are known to those skilled in the art and include, for example, coconut oil and palm kernel oil.

In some embodiments, the carrier comprises one or more polyesterdiols. The polyesterdiol may be a linear two to ten units polymer (also referred to as (ester)₂₋₁₀ glycol), that is derived from natural or non-natural sources such as vegetables, fruits, bacteria, yeast, algae, or manufactured by chemical processes.

For example, in some embodiments, the polyesterdiol is 1) a polypropylene glycol such as: dipropylene glycol; tripropylene glycol, including tetra-, penta-, hexa-, hepta-, octa-, nona- and decapropylene glycol and other derivatives thereof; 2) a polybutylene glycol such as: dibutylene glycol, tributylene glycol, including tetra-, penta-, hexa-, hepta-, octa-, nona- and decabutylene glycol, and other derivatives thereof: 3) also including 2-10 unit polymers of rare organic ester types such as pentylene, octylene, terpentylene, nonylene, linalylene, isoamylene, isobutylene, geranylene, bornylene, benzylene and allylene, caprylylene, such as, for example, polyisobutylene glycol such as diisobutylene glycol; and 4) triethylene glycol, including tetra-, penta-, hexa-, hepta-, octa-, nona- and decaethylene glycols and other derivatives thereof such as acid or sugar conjugates, and esters or ether or alcohol derivatives.

In some embodiments, the carrier is a linear polyesterdiol selected from the group consisting of: (ethylene)₂₋₁₀ glycol; (propylene)₂₋₁₀ glycol; (butylene)₂₋₁₀ glycol; (pentylene)₂₋₁₀ glycol; (octylene)₂₋₁₀ glycol; (terpentylene)₂₋₁₀ glycol; (nonylene)₂₋₁₀ glycol; (linalylene)₂₋₁₀ glycol; (isoamylene)₂₋₁₀ glycol; (isobutylene)₂₋₁₀ glycol; (geranylene)₂₋₁₀ glycol; (bornylene)₂₋₁₀ glycol; (benzylene)₂₋₁₀ glycol; (allylene)₂₋₁₀ glycol; and (caprylylene)₂₋₁₀ glycol; acid or sugar conjugates thereof, and ester or ether or alcohol derivatives thereof.

In some embodiments, the carrier is selected from the group consisting of: triethylene glycol; tetraethyleneglycol, pentaethylenglycol, hexaethyleneglycol, heptaethyleneglycol, octaethyleneglycol, nonaethylenglycol; decaethylene glycol; dipropylene glycol; tripropylene glycol; tetrapropylene glycol, pentapropylene glycol, hexapropylene glycol, heptapropylene glycol, octapropylene glycol, nonapropylene glycol; decapropylene glycol; dibutylene glycol, tributylene glycol; tetrabutylene glycol, pentabutylene glycol, hexabutylene glycol, heptabutylene glycol, octabutylene glycol, nonabutylene glycol, decabutylene glycol and diisobutylene glycol.

In some embodiments, the carrier is selected from the group consisting borneol, camphor, 1,8-Cineole, citral, geraniol, indomethacin, limonene, linalool, linalyl acetate, β-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone

In some embodiments, the ACP composition comprises one or more of propylene glycol, glycerine, vegetable glycerine, and/or water.

If desired, one or more flavorants may be added to the composition. Non-limiting examples include wintergreen, propolis, eucalyptus, tobacco, Turkish tobacco, menthol, peppermint, spearmint, green tea, cream, natural sweetener, banana, strawberry, blueberry, mango, raspberry, peach, pineapple, apple, orange, papaya, watermelon, grape, cherry, coffee, chocolate, vanilla, cola, piña colada, coconut, almond, cinnamon, white chocolate, rum, butterscotch, and caramel or the like.

ACP compositions of the disclosure can be free, essentially free, or substantially free from one or more other terpenes, such as free, essentially free, or substantially free from one or more of: terpinolene, alpha phelladrene, beta ocimene, carene, limonene, gamma terpinene, alpha pinene, alpha terpinene, beta pinene, fenchol, camphene, alpha terpineol, linalool, caryphyllene oxide, and/or myrcene.

ACP compositions of the disclosure can be free, essentially free, or substantially free from one or more other cannabinoids, such as free, essentially free, or substantially free from one or more of delta-9-tetrahydrocannabinol (THC) and/or cannabidiol (CBD).

ACP compositions of the disclosure can be free, essentially free, or substantially free from one or more carriers. For example, carriers that are not present in the ACP compositions can include borneol, camphor, 1,8-Cineole, citral, geraniol, indomethacin, limonene, linalool, linalyl acetate, β-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone.

In some embodiments, the ACP composition is administered via a handheld electronic device (ACP device) that vaporizes the ACP for inhalation by a user. In some embodiments, the ACP composition and/or ACP device do not include or contain additional constituents, carriers, or additives. For example, the ACP composition can be pure, essentially pure, or substantially pure, for example, having about 50% purity, about 55% purity, about 60% purity, about 65% purity, about 70% purity, about 75% purity, about 80% purity, about 85% purity, about 90% purity, about 95% purity, about 99% purity, or having greater than about 99% purity.

In some embodiments, the ACP composition is free from, essentially free from, or substantially free from one or more of: a carrier, propylene glycol, glycerine, vegetable glycerine, water, and/or flavorants. In some embodiments, the ACP composition is free from, essentially free from, or substantially free from one or more other terpenes and one or more other cannabinoids. It is to be understood that the percentages and purities discussed herein can be in weight or volume, depending on the embodiment and/or implementation.

In some embodiments, components of an ACP device include a mouthpiece, an ACP reservoir (which can be a tank or cartridge), a heating element or other atomizer (e.g., piezoelectric atomizer, inkjet-type atomizer, etc.), and an actuator (such as a switch or pressure sensor) to activate the heating element/atomizer. Depending on the embodiment, the ACP reservoir can be configured to store between about 0.5 mg and 500 mg of a ACP composition/ACP liquid, for example, between about 1 mg and about 250 mg, about 2 mg and 150 mg, about 3 mg and about 100 mg, about 4 mg and about 75 mg, about 5 mg and about 50 mg, or between about 10 mg and about 30 mg.

Depending on the embodiment, the ACP reservoir can be configured to store about 0.1 ml, about 0.5 ml, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, about 50 ml, about 55 ml, about 60 ml, about 70 ml, about 80 ml, about 90 ml, about 100 ml, or more than 100 ml.

Some implementations of the ACP device can include a microprocessor and a battery or other power source. Some implementations include and/or define a ACP vapor chamber or ACP atomization chamber. In some embodiments, an atomizer can include a small heater (e.g., heat coil or other heat element), that vaporizes a liquid ACP composition stored in the ACP reservoir. Some embodiments include a one or more wicks or wicking material configured to draw liquid ACP composition onto or into the heater or atomizer. In some embodiments, when a user activates an actuator (i.e., activates a pressure sensor by inhaling, presses a switch), the atomizer is activated (e.g., the heater is powered) and the liquid ACP composition is atomized or vaporized.

Depending on the embodiment and/or implementation, the heater and/or liquid ACP composition can reach a temperature from about 75° C. to about 350° C., from about 80° C. to about 300° C., from about 85° C. to about 275° C., from about 90° C. to about 250° C., from about 95° C. to about 225° C., from about 100° C. to about 200° C., from about 101° C. to about 175° C., from about 102° C. to about 150° C., from about 103° C. to about 125° C., from about 104° C. to about 115° C., or from about 105° C. to about 110° C. Some embodiments can include devices and/or configurations such as discussed in one or more of the following, including combinations thereof: U.S. Pat. App. Pub. No. 2017/0023235; U.S. Pat. App. Pub. No. 2015/0114411; U.S. Pat. App. Pub. No. 2016/0000149; U.S. Pat. App. Pub. No. 2016/0270451; U.S. Pat. App. Pub. No. 2016/0053988; U.S. Pat. App. Pub. No. 2015/0327595; U.S. Pat. App. Pub. No. 2014/0299125; U.S. Pat. App. Pub. No. 2014/0182608; U.S. Pat. App. Pub. No. 2014/0286630; WIPO Patent Application Publication WO/2016/156497; WIPO Patent Application Publication WO/2016/030369; U.S. Pat. Nos. 9,320,300; 9,095,175; 9,259,035; 9,326,549; 7,997,280; 9,459,021; 9,427,711; and/or U.S. Pat. No. 9,352,288; each of which is expressly incorporated by reference herein for all purposes.

In some embodiments, the atomizer of an ACP device of the disclosure is configured to vaporize an average dose per actuation such that an average dose can be inhaled by a single inhalation of a user.

Mixtures of Beta Caryophyllene and Alpha Caryophyllene

As detailed in the present disclosure, the Inventors have found beneficial and unexpected effects from administration of inhaled compositions of BCP and ACP.

According to some embodiments of the disclosure, there is provided low-dose vaporizable compositions of BCP and ACP for use in a human subject in need thereof.

According to an aspect of some embodiments of the disclosure, there is also provided a device configured to provide vaporized BCP and ACP compositions as a medicament, and in particular, a device configured to provide vaporized BCP and ACP as a treatment for a variety of conditions in a subject in need thereof.

In some embodiments, such a BCP and ACP composition is configured for administration to a human subject via inhalation. In some embodiments, such an BCP and ACP composition is configured for administration via aerosolization. In some embodiments, such an BCP and ACP composition is configured for administration via heat-based vaporization.

According to an aspect of some embodiments of the disclosure, there is also provided a method for treating a variety of conditions in a human subject in need thereof, the method comprising administering a low-dose aerosolized BCP and ACP composition to the subject via inhalation.

In some embodiments, the volume/volume ratio or the mass/mass ratio of BCP to ACP in a caryophyllene composition of the disclosure is about 1:1000, 1:500, 1:400, 1:300, 1:200, 1:150, 1:125, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or about 1:1 BCP to ACP.

In some embodiments, the volume/volume ratio or the mass/mass ratio of ACP to BCP in a caryophyllene composition of the disclosure is about 1:1000, 1:500, 1:400, 1:300, 1:200, 1:150, 1:125, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, or about 1:2 ACP to BCP.

The efficacy of the methods and caryophyllene compositions according to the teachings of the disclosure are discussed in the experimental section.

According to some embodiments, the compositions and methods of treatments disclosed herein are useful for treating one or more of dystonia, akathisia (as an anti-convulsant); glaucoma; ischemic disease (Alzheimer's, Parkinson's, Down Syndrome, HIV, dementia, etc.); for patients treated with oxidant-inducing agents for chemotherapy, radiation; motion sickness (anti-emetic); pain (chronic and acute); pain and inflammation (e.g., arthritis, etc.); seizure disorders; neurological pain (e.g., MS-related); weight-loss; depression, attention (for example, attention deficit disorder, attention deficit hyperactivity disorder), and other psychiatric disorders; sleep disorders (for example, insomnia, sleep apnea, Wittmaack Ekbom's Syndrome, nightmares); sleepiness; smoking habits, nicotine habits, and/or other addictions/addictive behaviors. For example, a user of inhaled low-dose caryophyllene can feel increased energy and can take the inhaled low-dose caryophyllene in place of a cigarette or nicotine. In some embodiments, the compositions, devices, and methods of treatments disclosed herein are useful in the treatment of at least one positive or negative symptom of one or more of dystonia, akathisia (anti-convulsant); glaucoma; ischemic disease (Alzheimer's, Parkinson's, Down Syndrome, HIV, dementia, etc.); for patients treated with oxidant-inducing agents for chemotherapy, radiation; motion sickness (anti-emetic); pain (chronic and acute); pain and inflammation (e.g., arthritis, etc.); seizure disorders; neurological pain (e.g., MS-related); weight-loss; depression, attention (for example, attention deficit disorder, attention deficit hyperactivity disorder), and other psychiatric disorders; sleep disorders (for example, insomnia, sleep apnea, Wittmaack Ekbom's Syndrome, nightmares); sleepiness; smoking habits, nicotine habits, and/or other addictions/addictive behaviors. The duration of treatment according to the methods of treatment of the disclosure can be any suitable duration as determined by a supervising health-care professional.

In some embodiments, a low dose vaporizable caryophyllene composition according to the disclosure is provided as or made as an inhalable dosage form.

In some embodiments of a method of the disclosure, the average dose of caryophyllene administered to a human subject via inhalation of a vapor thereof is from about 0.005 mg/kg to about 0.350 mg/kg, such as, for example, from about 0.006 mg/kg to about 0.340 mg/kg, from about 0.007 mg/kg to about 0.335 mg/kg, from about 0.008 mg/kg to about 0.330 mg/kg, from about 0.009 mg/kg to about 0.325 mg/kg, from about 0.01 mg/kg to about 0.320 mg/kg, from about 0.011 mg/kg to about 0.315 mg/kg, from about 0.012 mg/kg to about 0.310 mg/kg, from about 0.013 mg/kg to about 0.305 mg/kg, from about 0.014 mg/kg to about 0.300 mg/kg, from about 0.015 mg/kg to about 0.295 mg/kg, from about 0.016 mg/kg to about 0.290 mg/kg, from about 0.017 mg/kg to about 0.285 mg/kg, from about 0.018 mg/kg to about 0.280 mg/kg, from about 0.019 mg/kg to about 0.275 mg/kg, from about 0.020 mg/kg to about 0.270 mg/kg, from about 0.021 mg/kg to about 0.265 mg/kg, from about 0.022 mg/kg to about 0.260 mg/kg, from about 0.023 mg/kg to about 0.255 mg/kg, from about 0.024 mg/kg to about 0.250 mg/kg, from about 0.025 mg/kg to about 0.245 mg/kg, from about 0.026 mg/kg to about 0.240 mg/kg, from about 0.027 mg/kg to about 0.235 mg/kg, from about 0.028 mg/kg to about 0.230 mg/kg, from about 0.029 mg/kg to about 0.225 mg/kg, from about 0.030 mg/kg to about 0.220 mg/kg, from about 0.031 mg/kg to about 0.215 mg/kg, from about 0.032 mg/kg to about 0.210 mg/kg, from about 0.033 mg/kg to about 0.205 mg/kg, from about 0.034 mg/kg to about 0.200 mg/kg, from about 0.035 mg/kg to about 0.195 mg/kg, from about 0.036 mg/kg to about 0.190 mg/kg, from about 0.037 mg/kg to about 0.185 mg/kg, from about 0.038 mg/kg to about 0.180 mg/kg, from about 0.039 mg/kg to about 0.175 mg/kg, from about 0.040 mg/kg to about 0.170 mg/kg, from about 0.045 mg/kg to about 0.165 mg/kg, from about 0.050 mg/kg to about 0.160 mg/kg, from about 0.055 mg/kg to about 0.155 mg/kg, from about 0.060 mg/kg to about 0.150 mg/kg, from about 0.065 mg/kg to about 0.145 mg/kg, from about 0.070 mg/kg to about 0.140 mg/kg, from about 0.075 mg/kg to about 0.135 mg/kg, from about 0.080 mg/kg to about 0.130 mg/kg, from about 0.085 mg/kg to about 0.125 mg/kg, from about 0.090 mg/kg to about 0.120 mg/kg, from about 0.095 mg/kg to about 0.115 mg/kg, or from about 0.100 mg/kg to about 0.110 mg/kg.

In some embodiments, the average dose of caryophyllene is about 0.001 mg, about 0.005 mg, about 0.01 mg, about 0.015 mg, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.10 mg, about 0.105 mg, about 0.11 mg, about 0.115 mg, about 0.12 mg, about 0.125 mg, about 0.13 mg, about 0.135 mg, about 0.14 mg, about 0.145 mg, about 0.15 mg, about 0.155 mg, about 0.16 mg, about 0.165 mg, about 0.17 mg, about 0.175 mg, about 0.18 mg, about 0.185 mg, about 0.19 mg, about 0.195 mg, about 0.20 mg, about 0.205 mg, about 0.21 mg, about 0.215 mg, about 0.22 mg, about 0.225 mg, about 0.23 mg, about 0.235 mg, about 0.24 mg, about 0.245 mg, about 0.25 mg, about 0.255 mg, about 0.26 mg, about 0.265 mg, about 0.27 mg, about 0.275 mg, about 0.28 mg, about 0.285 mg, about 0.29 mg, about 0.295 mg, about 0.30 mg, about 0.305 mg, about 0.31 mg, about 0.315 mg, about 0.32 mg, about 0.325 mg, about 0.33 mg, about 0.335 mg, about 0.34 mg, about 0.345 mg, about 0.35 mg, about 0.355 mg, about 0.36 mg, about 0.365 mg, about 0.37 mg, about 0.375 mg, about 0.38 mg, about 0.385 mg, about 0.39 mg, about 0.395 mg, about 0.40 mg, about 0.405 mg, about 0.41 mg, about 0.415 mg, about 0.42 mg, about 0.425 mg, about 0.43 mg, about 0.435 mg, about 0.44 mg, about 0.445 mg, about 0.45 mg, about 0.455 mg, about 0.46 mg, about 0.465 mg, about 0.47 mg, about 0.475 mg, about 0.48 mg, about 0.485 mg, about 0.49 mg, about 0.495 mg, about 0.50 mg, about 0.505 mg, about 0.51 mg, about 0.515 mg, about 0.52 mg, about 0.525 mg, about 0.53 mg, about 0.535 mg, about 0.54 mg, about 0.545 mg, about 0.55 mg, about 0.555 mg, about 0.56 mg, about 0.565 mg, about 0.57 mg, about 0.575 mg, about 0.58 mg, about 0.585 mg, about 0.59 mg, about 0.595 mg, about 0.60 mg, about 0.605 mg, about 0.61 mg, about 0.615 mg, about 0.62 mg, about 0.625 mg, about 0.63 mg, about 0.635 mg, about 0.64 mg, about 0.645 mg, about 0.65 mg, about 0.655 mg, about 0.66 mg, about 0.665 mg, about 0.67 mg, about 0.675 mg, about 0.68 mg, about 0.685 mg, about 0.69 mg, about 0.695 mg, about 0.70 mg, about 0.705 mg, about 0.71 mg, about 0.715 mg, about 0.72 mg, about 0.725 mg, about 0.73 mg, about 0.735 mg, about 0.74 mg, about 0.745 mg, about 0.75 mg, about 0.755 mg, about 0.76 mg, about 0.765 mg, about 0.77 mg, about 0.775 mg, about 0.78 mg, about 0.785 mg, about 0.79 mg, about 0.795 mg, about 0.80 mg, about 0.805 mg, about 0.81 mg, about 0.815 mg, about 0.82 mg, about 0.825 mg, about 0.83 mg, about 0.835 mg, about 0.84 mg, about 0.845 mg, about 0.85 mg, about 0.855 mg, about 0.86 mg, about 0.865 mg, about 0.87 mg, about 0.875 mg, about 0.88 mg, about 0.885 mg, about 0.89 mg, about 0.895 mg, about 0.90 mg, about 0.905 mg, about 0.91 mg, about 0.915 mg, about 0.92 mg, about 0.925 mg, about 0.93 mg, about 0.935 mg, about 0.94 mg, about 0.945 mg, about 0.95 mg, about 0.955 mg, about 0.96 mg, about 0.965 mg, about 0.97 mg, about 0.975 mg, about 0.98 mg, about 0.985 mg, about 0.99 mg, or about 0.995 mg.

In some embodiments, the average dose of caryophyllene is about 1.00 mg, about, about 1.005 mg, about 1.01 mg, about 1.015 mg, about 1.02 mg, about 1.025 mg, about 1.03 mg, about 1.035 mg, about 1.04 mg, about 1.045 mg, about 1.05 mg, about 1.055 mg, about 1.06 mg, about 1.065 mg, about 1.07 mg, about 1.075 mg, about 1.08 mg, about 1.085 mg, about 1.09 mg, about 1.095 mg, about 1.10 mg, about 1.105 mg, about 1.11 mg, about 1.115 mg, about 1.12 mg, about 1.125 mg, about 1.13 mg, about 1.135 mg, about 1.14 mg, about 1.145 mg, about 1.15 mg, about 1.155 mg, about 1.16 mg, about 1.165 mg, about 1.17 mg, about 1.175 mg, about 1.18 mg, about 1.185 mg, about 1.19 mg, about 1.195 mg, about 1.20 mg, about 1.205 mg, about 1.21 mg, about 1.215 mg, about 1.22 mg, about 1.225 mg, about 1.23 mg, about 1.235 mg, about 1.24 mg, about 1.245 mg, about 1.25 mg, about 1.255 mg, about 1.26 mg, about 1.265 mg, about 1.27 mg, about 1.275 mg, about 1.28 mg, about 1.285 mg, about 1.29 mg, about 1.295 mg, about 1.30 mg, about 1.305 mg, about 1.31 mg, about 1.315 mg, about 1.32 mg, about 1.325 mg, about 1.33 mg, about 1.335 mg, about 1.34 mg, about 1.345 mg, about 1.35 mg, about 1.355 mg, about 1.36 mg, about 1.365 mg, about 1.37 mg, about 1.375 mg, about 1.38 mg, about 1.385 mg, about 1.39 mg, about 1.395 mg, about 1.40 mg, about 1.405 mg, about 1.41 mg, about 1.415 mg, about 1.42 mg, about 1.425 mg, about 1.43 mg, about 1.435 mg, about 1.44 mg, about 1.445 mg, about 1.45 mg, about 1.455 mg, about 1.46 mg, about 1.465 mg, about 1.47 mg, about 1.475 mg, about 1.48 mg, about 1.485 mg, about 1.49 mg, about 1.495 mg, about 1.50 mg, about 1.505 mg, about 1.51 mg, about 1.515 mg, about 1.52 mg, about 1.525 mg, about 1.53 mg, about 1.535 mg, about 1.54 mg, about 1.545 mg, about 1.55 mg, about 1.555 mg, about 1.56 mg, about 1.565 mg, about 1.57 mg, about 1.575 mg, about 1.58 mg, about 1.585 mg, about 1.59 mg, about 1.595 mg, about 1.60 mg, about 1.605 mg, about 1.61 mg, about 1.615 mg, about 1.62 mg, about 1.625 mg, about 1.63 mg, about 1.635 mg, about 1.64 mg, about 1.645 mg, about 1.65 mg, about 1.655 mg, about 1.66 mg, about 1.665 mg, about 1.67 mg, about 1.675 mg, about 1.68 mg, about 1.685 mg, about 1.69 mg, about 1.695 mg, about 1.70 mg, about 1.705 mg, about 1.71 mg, about 1.715 mg, about 1.72 mg, about 1.725 mg, about 1.73 mg, about 1.735 mg, about 1.74 mg, about 1.745 mg, about 1.75 mg, about 1.755 mg, about 1.76 mg, about 1.765 mg, about 1.77 mg, about 1.775 mg, about 1.78 mg, about 1.785 mg, about 1.79 mg, about 1.795 mg, about 1.80 mg, about 1.805 mg, about 1.81 mg, about 1.815 mg, about 1.82 mg, about 1.825 mg, about 1.83 mg, about 1.835 mg, about 1.84 mg, about 1.845 mg, about 1.85 mg, about 1.855 mg, about 1.86 mg, about 1.865 mg, about 1.87 mg, about 1.875 mg, about 1.88 mg, about 1.885 mg, about 1.89 mg, about 1.895 mg, about 1.90 mg, about 1.905 mg, about 1.91 mg, about 1.915 mg, about 1.92 mg, about 1.925 mg, about 1.93 mg, about 1.935 mg, about 1.94 mg, about 1.945 mg, about 1.95 mg, about 1.955 mg, about 1.96 mg, about 1.965 mg, about 1.97 mg, about 1.975 mg, about 1.98 mg, about 1.985 mg, about 1.99 mg, or about 1.995 mg.

In some embodiments, the average dose of caryophyllene is about 2.00 mg, about, about 2.005 mg, about 2.01 mg, about 2.015 mg, about 2.02 mg, about 2.025 mg, about 2.03 mg, about 2.035 mg, about 2.04 mg, about 2.045 mg, about 2.05 mg, about 2.055 mg, about 2.06 mg, about 2.065 mg, about 2.07 mg, about 2.075 mg, about 2.08 mg, about 2.085 mg, about 2.09 mg, about 2.095 mg, about 2.10 mg, about 2.105 mg, about 2.11 mg, about 2.115 mg, about 2.12 mg, about 2.125 mg, about 2.13 mg, about 2.135 mg, about 2.14 mg, about 2.145 mg, about 2.15 mg, about 2.155 mg, about 2.16 mg, about 2.165 mg, about 2.17 mg, about 2.175 mg, about 2.18 mg, about 2.185 mg, about 2.19 mg, about 2.195 mg, about 2.20 mg, about 2.205 mg, about 2.21 mg, about 2.215 mg, about 2.22 mg, about 2.225 mg, about 2.23 mg, about 2.235 mg, about 2.24 mg, about 2.245 mg, about 2.25 mg, about 2.255 mg, about 2.26 mg, about 2.265 mg, about 2.27 mg, about 2.275 mg, about 2.28 mg, about 2.285 mg, about 2.29 mg, about 2.295 mg, about 2.30 mg, about 2.305 mg, about 2.31 mg, about 2.315 mg, about 2.32 mg, about 2.325 mg, about 2.33 mg, about 2.335 mg, about 2.34 mg, about 2.345 mg, about 2.35 mg, about 2.355 mg, about 2.36 mg, about 2.365 mg, about 2.37 mg, about 2.375 mg, about 2.38 mg, about 2.385 mg, about 2.39 mg, about 2.395 mg, about 2.40 mg, about 2.405 mg, about 2.41 mg, about 2.415 mg, about 2.42 mg, about 2.425 mg, about 2.43 mg, about 2.435 mg, about 2.44 mg, about 2.445 mg, about 2.45 mg, about 2.455 mg, about 2.46 mg, about 2.465 mg, about 2.47 mg, about 2.475 mg, about 2.48 mg, about 2.485 mg, about 2.49 mg, about 2.495 mg, about 2.50 mg, about 2.505 mg, about 2.51 mg, about 2.515 mg, about 2.52 mg, about 2.525 mg, about 2.53 mg, about 2.535 mg, about 2.54 mg, about 2.545 mg, about 2.55 mg, about 2.555 mg, about 2.56 mg, about 2.565 mg, about 2.57 mg, about 2.575 mg, about 2.58 mg, about 2.585 mg, about 2.59 mg, about 2.595 mg, about 2.60 mg, about 2.605 mg, about 2.61 mg, about 2.615 mg, about 2.62 mg, about 2.625 mg, about 2.63 mg, about 2.635 mg, about 2.64 mg, about 2.645 mg, about 2.65 mg, about 2.655 mg, about 2.66 mg, about 2.665 mg, about 2.67 mg, about 2.675 mg, about 2.68 mg, about 2.685 mg, about 2.69 mg, about 2.695 mg, about 2.70 mg, about 2.705 mg, about 2.71 mg, about 2.715 mg, about 2.72 mg, about 2.725 mg, about 2.73 mg, about 2.735 mg, about 2.74 mg, about 2.745 mg, about 2.75 mg, about 2.755 mg, about 2.76 mg, about 2.765 mg, about 2.77 mg, about 2.775 mg, about 2.78 mg, about 2.785 mg, about 2.79 mg, about 2.795 mg, about 2.80 mg, about 2.805 mg, about 2.81 mg, about 2.815 mg, about 2.82 mg, about 2.825 mg, about 2.83 mg, about 2.835 mg, about 2.84 mg, about 2.845 mg, about 2.85 mg, about 2.855 mg, about 2.86 mg, about 2.865 mg, about 2.87 mg, about 2.875 mg, about 2.88 mg, about 2.885 mg, about 2.89 mg, about 2.895 mg, about 2.90 mg, about 2.905 mg, about 2.91 mg, about 2.915 mg, about 2.92 mg, about 2.925 mg, about 2.93 mg, about 2.935 mg, about 2.94 mg, about 2.945 mg, about 2.95 mg, about 2.955 mg, about 2.96 mg, about 2.965 mg, about 2.97 mg, about 2.975 mg, about 2.98 mg, about 2.985 mg, about 2.99 mg, or about 2.995 mg.

In some embodiments, the average dose of caryophyllene is about 3.00 mg, about, about 3.005 mg, about 3.01 mg, about 3.015 mg, about 3.02 mg, about 3.025 mg, about 3.03 mg, about 3.035 mg, about 3.04 mg, about 3.045 mg, about 3.05 mg, about 3.055 mg, about 3.06 mg, about 3.065 mg, about 3.07 mg, about 3.075 mg, about 3.08 mg, about 3.085 mg, about 3.09 mg, about 3.095 mg, about 3.10 mg, about 3.105 mg, about 3.11 mg, about 3.115 mg, about 3.12 mg, about 3.125 mg, about 3.13 mg, about 3.135 mg, about 3.14 mg, about 3.145 mg, about 3.15 mg, about 3.155 mg, about 3.16 mg, about 3.165 mg, about 3.17 mg, about 3.175 mg, about 3.18 mg, about 3.185 mg, about 3.19 mg, about 3.195 mg, about 3.20 mg, about 3.205 mg, about 3.21 mg, about 3.215 mg, about 3.22 mg, about 3.225 mg, about 3.23 mg, about 3.235 mg, about 3.24 mg, about 3.245 mg, about 3.25 mg, about 3.255 mg, about 3.26 mg, about 3.265 mg, about 3.27 mg, about 3.275 mg, about 3.28 mg, about 3.285 mg, about 3.29 mg, about 3.295 mg, about 3.30 mg, about 3.305 mg, about 3.31 mg, about 3.315 mg, about 3.32 mg, about 3.325 mg, about 3.33 mg, about 3.335 mg, about 3.34 mg, about 3.345 mg, about 3.35 mg, about 3.355 mg, about 3.36 mg, about 3.365 mg, about 3.37 mg, about 3.375 mg, about 3.38 mg, about 3.385 mg, about 3.39 mg, about 3.395 mg, about 3.40 mg, about 3.405 mg, about 3.41 mg, about 3.415 mg, about 3.42 mg, about 3.425 mg, about 3.43 mg, about 3.435 mg, about 3.44 mg, about 3.445 mg, about 3.45 mg, about 3.455 mg, about 3.46 mg, about 3.465 mg, about 3.47 mg, about 3.475 mg, about 3.48 mg, about 3.485 mg, about 3.49 mg, about 3.495 mg, about 3.50 mg, about 3.505 mg, about 3.51 mg, about 3.515 mg, about 3.52 mg, about 3.525 mg, about 3.53 mg, about 3.535 mg, about 3.54 mg, about 3.545 mg, about 3.55 mg, about 3.555 mg, about 3.56 mg, about 3.565 mg, about 3.57 mg, about 3.575 mg, about 3.58 mg, about 3.585 mg, about 3.59 mg, about 3.595 mg, about 3.60 mg, about 3.605 mg, about 3.61 mg, about 3.615 mg, about 3.62 mg, about 3.625 mg, about 3.63 mg, about 3.635 mg, about 3.64 mg, about 3.645 mg, about 3.65 mg, about 3.655 mg, about 3.66 mg, about 3.665 mg, about 3.67 mg, about 3.675 mg, about 3.68 mg, about 3.685 mg, about 3.69 mg, about 3.695 mg, about 3.70 mg, about 3.705 mg, about 3.71 mg, about 3.715 mg, about 3.72 mg, about 3.725 mg, about 3.73 mg, about 3.735 mg, about 3.74 mg, about 3.745 mg, about 3.75 mg, about 3.755 mg, about 3.76 mg, about 3.765 mg, about 3.77 mg, about 3.775 mg, about 3.78 mg, about 3.785 mg, about 3.79 mg, about 3.795 mg, about 3.80 mg, about 3.805 mg, about 3.81 mg, about 3.815 mg, about 3.82 mg, about 3.825 mg, about 3.83 mg, about 3.835 mg, about 3.84 mg, about 3.845 mg, about 3.85 mg, about 3.855 mg, about 3.86 mg, about 3.865 mg, about 3.87 mg, about 3.875 mg, about 3.88 mg, about 3.885 mg, about 3.89 mg, about 3.895 mg, about 3.90 mg, about 3.905 mg, about 3.91 mg, about 3.915 mg, about 3.92 mg, about 3.925 mg, about 3.93 mg, about 3.935 mg, about 3.94 mg, about 3.945 mg, about 3.95 mg, about 3.955 mg, about 3.96 mg, about 3.965 mg, about 3.97 mg, about 3.975 mg, about 3.98 mg, about 3.985 mg, about 3.99 mg, or about 3.995 mg.

In some embodiments, the average dose of caryophyllene is about 4.00 mg, about, about 4.005 mg, about 4.01 mg, about 4.015 mg, about 4.02 mg, about 4.025 mg, about 4.03 mg, about 4.035 mg, about 4.04 mg, about 4.045 mg, about 4.05 mg, about 4.055 mg, about 4.06 mg, about 4.065 mg, about 4.07 mg, about 4.075 mg, about 4.08 mg, about 4.085 mg, about 4.09 mg, about 4.095 mg, about 4.10 mg, about 4.105 mg, about 4.11 mg, about 4.115 mg, about 4.12 mg, about 4.125 mg, about 4.13 mg, about 4.135 mg, about 4.14 mg, about 4.145 mg, about 4.15 mg, about 4.155 mg, about 4.16 mg, about 4.165 mg, about 4.17 mg, about 4.175 mg, about 4.18 mg, about 4.185 mg, about 4.19 mg, about 4.195 mg, about 4.20 mg, about 4.205 mg, about 4.21 mg, about 4.215 mg, about 4.22 mg, about 4.225 mg, about 4.23 mg, about 4.235 mg, about 4.24 mg, about 4.245 mg, about 4.25 mg, about 4.255 mg, about 4.26 mg, about 4.265 mg, about 4.27 mg, about 4.275 mg, about 4.28 mg, about 4.285 mg, about 4.29 mg, about 4.295 mg, about 4.30 mg, about 4.305 mg, about 4.31 mg, about 4.315 mg, about 4.32 mg, about 4.325 mg, about 4.33 mg, about 4.335 mg, about 4.34 mg, about 4.345 mg, about 4.35 mg, about 4.355 mg, about 4.36 mg, about 4.365 mg, about 4.37 mg, about 4.375 mg, about 4.38 mg, about 4.385 mg, about 4.39 mg, about 4.395 mg, about 4.40 mg, about 4.405 mg, about 4.41 mg, about 4.415 mg, about 4.42 mg, about 4.425 mg, about 4.43 mg, about 4.435 mg, about 4.44 mg, about 4.445 mg, about 4.45 mg, about 4.455 mg, about 4.46 mg, about 4.465 mg, about 4.47 mg, about 4.475 mg, about 4.48 mg, about 4.485 mg, about 4.49 mg, about 4.495 mg, about 4.50 mg, about 4.505 mg, about 4.51 mg, about 4.515 mg, about 4.52 mg, about 4.525 mg, about 4.53 mg, about 4.535 mg, about 4.54 mg, about 4.545 mg, about 4.55 mg, about 4.555 mg, about 4.56 mg, about 4.565 mg, about 4.57 mg, about 4.575 mg, about 4.58 mg, about 4.585 mg, about 4.59 mg, about 4.595 mg, about 4.60 mg, about 4.605 mg, about 4.61 mg, about 4.615 mg, about 4.62 mg, about 4.625 mg, about 4.63 mg, about 4.635 mg, about 4.64 mg, about 4.645 mg, about 4.65 mg, about 4.655 mg, about 4.66 mg, about 4.665 mg, about 4.67 mg, about 4.675 mg, about 4.68 mg, about 4.685 mg, about 4.69 mg, about 4.695 mg, about 4.70 mg, about 4.705 mg, about 4.71 mg, about 4.715 mg, about 4.72 mg, about 4.725 mg, about 4.73 mg, about 4.735 mg, about 4.74 mg, about 4.745 mg, about 4.75 mg, about 4.755 mg, about 4.76 mg, about 4.765 mg, about 4.77 mg, about 4.775 mg, about 4.78 mg, about 4.785 mg, about 4.79 mg, about 4.795 mg, about 4.80 mg, about 4.805 mg, about 4.81 mg, about 4.815 mg, about 4.82 mg, about 4.825 mg, about 4.83 mg, about 4.835 mg, about 4.84 mg, about 4.845 mg, about 4.85 mg, about 4.855 mg, about 4.86 mg, about 4.865 mg, about 4.87 mg, about 4.875 mg, about 4.88 mg, about 4.885 mg, about 4.89 mg, about 4.895 mg, about 4.90 mg, about 4.905 mg, about 4.91 mg, about 4.915 mg, about 4.92 mg, about 4.925 mg, about 4.93 mg, about 4.935 mg, about 4.94 mg, about 4.945 mg, about 4.95 mg, about 4.955 mg, about 4.96 mg, about 4.965 mg, about 4.97 mg, about 4.975 mg, about 4.98 mg, about 4.985 mg, about 4.99 mg, or about 4.995 mg.

In some embodiments, the average dose of caryophyllene is about 5.00 mg, about, about 5.005 mg, about 5.01 mg, about 5.015 mg, about 5.02 mg, about 5.025 mg, about 5.03 mg, about 5.035 mg, about 5.04 mg, about 5.045 mg, about 5.05 mg, about 5.055 mg, about 5.06 mg, about 5.065 mg, about 5.07 mg, about 5.075 mg, about 5.08 mg, about 5.085 mg, about 5.09 mg, about 5.095 mg, about 5.10 mg, about 5.105 mg, about 5.11 mg, about 5.115 mg, about 5.12 mg, about 5.125 mg, about 5.13 mg, about 5.135 mg, about 5.14 mg, about 5.145 mg, about 5.15 mg, about 5.155 mg, about 5.16 mg, about 5.165 mg, about 5.17 mg, about 5.175 mg, about 5.18 mg, about 5.185 mg, about 5.19 mg, about 5.195 mg, about 5.20 mg, about 5.205 mg, about 5.21 mg, about 5.215 mg, about 5.22 mg, about 5.225 mg, about 5.23 mg, about 5.235 mg, about 5.24 mg, about 5.245 mg, about 5.25 mg, about 5.255 mg, about 5.26 mg, about 5.265 mg, about 5.27 mg, about 5.275 mg, about 5.28 mg, about 5.285 mg, about 5.29 mg, about 5.295 mg, about 5.30 mg, about 5.305 mg, about 5.31 mg, about 5.315 mg, about 5.32 mg, about 5.325 mg, about 5.33 mg, about 5.335 mg, about 5.34 mg, about 5.345 mg, about 5.35 mg, about 5.355 mg, about 5.36 mg, about 5.365 mg, about 5.37 mg, about 5.375 mg, about 5.38 mg, about 5.385 mg, about 5.39 mg, about 5.395 mg, about 5.40 mg, about 5.405 mg, about 5.41 mg, about 5.415 mg, about 5.42 mg, about 5.425 mg, about 5.43 mg, about 5.435 mg, about 5.44 mg, about 5.445 mg, about 5.45 mg, about 5.455 mg, about 5.46 mg, about 5.465 mg, about 5.47 mg, about 5.475 mg, about 5.48 mg, about 5.485 mg, about 5.49 mg, about 5.495 mg, about 5.50 mg, about 5.505 mg, about 5.51 mg, about 5.515 mg, about 5.52 mg, about 5.525 mg, about 5.53 mg, about 5.535 mg, about 5.54 mg, about 5.545 mg, about 5.55 mg, about 5.555 mg, about 5.56 mg, about 5.565 mg, about 5.57 mg, about 5.575 mg, about 5.58 mg, about 5.585 mg, about 5.59 mg, about 5.595 mg, about 5.60 mg, about 5.605 mg, about 5.61 mg, about 5.615 mg, about 5.62 mg, about 5.625 mg, about 5.63 mg, about 5.635 mg, about 5.64 mg, about 5.645 mg, about 5.65 mg, about 5.655 mg, about 5.66 mg, about 5.665 mg, about 5.67 mg, about 5.675 mg, about 5.68 mg, about 5.685 mg, about 5.69 mg, about 5.695 mg, about 5.70 mg, about 5.705 mg, about 5.71 mg, about 5.715 mg, about 5.72 mg, about 5.725 mg, about 5.73 mg, about 5.735 mg, about 5.74 mg, about 5.745 mg, about 5.75 mg, about 5.755 mg, about 5.76 mg, about 5.765 mg, about 5.77 mg, about 5.775 mg, about 5.78 mg, about 5.785 mg, about 5.79 mg, about 5.795 mg, about 5.80 mg, about 5.805 mg, about 5.81 mg, about 5.815 mg, about 5.82 mg, about 5.825 mg, about 5.83 mg, about 5.835 mg, about 5.84 mg, about 5.845 mg, about 5.85 mg, about 5.855 mg, about 5.86 mg, about 5.865 mg, about 5.87 mg, about 5.875 mg, about 5.88 mg, about 5.885 mg, about 5.89 mg, about 5.895 mg, about 5.90 mg, about 5.905 mg, about 5.91 mg, about 5.915 mg, about 5.92 mg, about 5.925 mg, about 5.93 mg, about 5.935 mg, about 5.94 mg, about 5.945 mg, about 5.95 mg, about 5.955 mg, about 5.96 mg, about 5.965 mg, about 5.97 mg, about 5.975 mg, about 5.98 mg, about 5.985 mg, about 5.99 mg, or about 5.995 mg.

In some embodiments, the average dose of caryophyllene is about 6.00 mg, about, about 6.005 mg, about 6.01 mg, about 6.015 mg, about 6.02 mg, about 6.025 mg, about 6.03 mg, about 6.035 mg, about 6.04 mg, about 6.045 mg, about 6.05 mg, about 6.055 mg, about 6.06 mg, about 6.065 mg, about 6.07 mg, about 6.075 mg, about 6.08 mg, about 6.085 mg, about 6.09 mg, about 6.095 mg, about 6.10 mg, about 6.105 mg, about 6.11 mg, about 6.115 mg, about 6.12 mg, about 6.125 mg, about 6.13 mg, about 6.135 mg, about 6.14 mg, about 6.145 mg, about 6.15 mg, about 6.155 mg, about 6.16 mg, about 6.165 mg, about 6.17 mg, about 6.175 mg, about 6.18 mg, about 6.185 mg, about 6.19 mg, about 6.195 mg, about 6.20 mg, about 6.205 mg, about 6.21 mg, about 6.215 mg, about 6.22 mg, about 6.225 mg, about 6.23 mg, about 6.235 mg, about 6.24 mg, about 6.245 mg, about 6.25 mg, about 6.255 mg, about 6.26 mg, about 6.265 mg, about 6.27 mg, about 6.275 mg, about 6.28 mg, about 6.285 mg, about 6.29 mg, about 6.295 mg, about 6.30 mg, about 6.305 mg, about 6.31 mg, about 6.315 mg, about 6.32 mg, about 6.325 mg, about 6.33 mg, about 6.335 mg, about 6.34 mg, about 6.345 mg, about 6.35 mg, about 6.355 mg, about 6.36 mg, about 6.365 mg, about 6.37 mg, about 6.375 mg, about 6.38 mg, about 6.385 mg, about 6.39 mg, about 6.395 mg, about 6.40 mg, about 6.405 mg, about 6.41 mg, about 6.415 mg, about 6.42 mg, about 6.425 mg, about 6.43 mg, about 6.435 mg, about 6.44 mg, about 6.445 mg, about 6.45 mg, about 6.455 mg, about 6.46 mg, about 6.465 mg, about 6.47 mg, about 6.475 mg, about 6.48 mg, about 6.485 mg, about 6.49 mg, about 6.495 mg, about 6.50 mg, about 6.505 mg, about 6.51 mg, about 6.515 mg, about 6.52 mg, about 6.525 mg, about 6.53 mg, about 6.535 mg, about 6.54 mg, about 6.545 mg, about 6.55 mg, about 6.555 mg, about 6.56 mg, about 6.565 mg, about 6.57 mg, about 6.575 mg, about 6.58 mg, about 6.585 mg, about 6.59 mg, about 6.595 mg, about 6.60 mg, about 6.605 mg, about 6.61 mg, about 6.615 mg, about 6.62 mg, about 6.625 mg, about 6.63 mg, about 6.635 mg, about 6.64 mg, about 6.645 mg, about 6.65 mg, about 6.655 mg, about 6.66 mg, about 6.665 mg, about 6.67 mg, about 6.675 mg, about 6.68 mg, about 6.685 mg, about 6.69 mg, about 6.695 mg, about 6.70 mg, about 6.705 mg, about 6.71 mg, about 6.715 mg, about 6.72 mg, about 6.725 mg, about 6.73 mg, about 6.735 mg, about 6.74 mg, about 6.745 mg, about 6.75 mg, about 6.755 mg, about 6.76 mg, about 6.765 mg, about 6.77 mg, about 6.775 mg, about 6.78 mg, about 6.785 mg, about 6.79 mg, about 6.795 mg, about 6.80 mg, about 6.805 mg, about 6.81 mg, about 6.815 mg, about 6.82 mg, about 6.825 mg, about 6.83 mg, about 6.835 mg, about 6.84 mg, about 6.845 mg, about 6.85 mg, about 6.855 mg, about 6.86 mg, about 6.865 mg, about 6.87 mg, about 6.875 mg, about 6.88 mg, about 6.885 mg, about 6.89 mg, about 6.895 mg, about 6.90 mg, about 6.905 mg, about 6.91 mg, about 6.915 mg, about 6.92 mg, about 6.925 mg, about 6.93 mg, about 6.935 mg, about 6.94 mg, about 6.945 mg, about 6.95 mg, about 6.955 mg, about 6.96 mg, about 6.965 mg, about 6.97 mg, about 6.975 mg, about 6.98 mg, about 6.985 mg, about 6.99 mg, or about 6.995 mg.

In some embodiments, the average dose of caryophyllene is about 7.00 mg, about, about 7.005 mg, about 7.01 mg, about 7.015 mg, about 7.02 mg, about 7.025 mg, about 7.03 mg, about 7.035 mg, about 7.04 mg, about 7.045 mg, about 7.05 mg, about 7.055 mg, about 7.06 mg, about 7.065 mg, about 7.07 mg, about 7.075 mg, about 7.08 mg, about 7.085 mg, about 7.09 mg, about 7.095 mg, about 7.10 mg, about 7.105 mg, about 7.11 mg, about 7.115 mg, about 7.12 mg, about 7.125 mg, about 7.13 mg, about 7.135 mg, about 7.14 mg, about 7.145 mg, about 7.15 mg, about 7.155 mg, about 7.16 mg, about 7.165 mg, about 7.17 mg, about 7.175 mg, about 7.18 mg, about 7.185 mg, about 7.19 mg, about 7.195 mg, about 7.20 mg, about 7.205 mg, about 7.21 mg, about 7.215 mg, about 7.22 mg, about 7.225 mg, about 7.23 mg, about 7.235 mg, about 7.24 mg, about 7.245 mg, about 7.25 mg, about 7.255 mg, about 7.26 mg, about 7.265 mg, about 7.27 mg, about 7.275 mg, about 7.28 mg, about 7.285 mg, about 7.29 mg, about 7.295 mg, about 7.30 mg, about 7.305 mg, about 7.31 mg, about 7.315 mg, about 7.32 mg, about 7.325 mg, about 7.33 mg, about 7.335 mg, about 7.34 mg, about 7.345 mg, about 7.35 mg, about 7.355 mg, about 7.36 mg, about 7.365 mg, about 7.37 mg, about 7.375 mg, about 7.38 mg, about 7.385 mg, about 7.39 mg, about 7.395 mg, about 7.40 mg, about 7.405 mg, about 7.41 mg, about 7.415 mg, about 7.42 mg, about 7.425 mg, about 7.43 mg, about 7.435 mg, about 7.44 mg, about 7.445 mg, about 7.45 mg, about 7.455 mg, about 7.46 mg, about 7.465 mg, about 7.47 mg, about 7.475 mg, about 7.48 mg, about 7.485 mg, about 7.49 mg, about 7.495 mg, about 7.50 mg, about 7.505 mg, about 7.51 mg, about 7.515 mg, about 7.52 mg, about 7.525 mg, about 7.53 mg, about 7.535 mg, about 7.54 mg, about 7.545 mg, about 7.55 mg, about 7.555 mg, about 7.56 mg, about 7.565 mg, about 7.57 mg, about 7.575 mg, about 7.58 mg, about 7.585 mg, about 7.59 mg, about 7.595 mg, about 7.60 mg, about 7.605 mg, about 7.61 mg, about 7.615 mg, about 7.62 mg, about 7.625 mg, about 7.63 mg, about 7.635 mg, about 7.64 mg, about 7.645 mg, about 7.65 mg, about 7.655 mg, about 7.66 mg, about 7.665 mg, about 7.67 mg, about 7.675 mg, about 7.68 mg, about 7.685 mg, about 7.69 mg, about 7.695 mg, about 7.70 mg, about 7.705 mg, about 7.71 mg, about 7.715 mg, about 7.72 mg, about 7.725 mg, about 7.73 mg, about 7.735 mg, about 7.74 mg, about 7.745 mg, about 7.75 mg, about 7.755 mg, about 7.76 mg, about 7.765 mg, about 7.77 mg, about 7.775 mg, about 7.78 mg, about 7.785 mg, about 7.79 mg, about 7.795 mg, about 7.80 mg, about 7.805 mg, about 7.81 mg, about 7.815 mg, about 7.82 mg, about 7.825 mg, about 7.83 mg, about 7.835 mg, about 7.84 mg, about 7.845 mg, about 7.85 mg, about 7.855 mg, about 7.86 mg, about 7.865 mg, about 7.87 mg, about 7.875 mg, about 7.88 mg, about 7.885 mg, about 7.89 mg, about 7.895 mg, about 7.90 mg, about 7.905 mg, about 7.91 mg, about 7.915 mg, about 7.92 mg, about 7.925 mg, about 7.93 mg, about 7.935 mg, about 7.94 mg, about 7.945 mg, about 7.95 mg, about 7.955 mg, about 7.96 mg, about 7.965 mg, about 7.97 mg, about 7.975 mg, about 7.98 mg, about 7.985 mg, about 7.99 mg, or about 7.995 mg.

In some embodiments, the average dose of caryophyllene composition is about 8.00 mg, about, about 8.005 mg, about 8.01 mg, about 8.015 mg, about 8.02 mg, about 8.025 mg, about 8.03 mg, about 8.035 mg, about 8.04 mg, about 8.045 mg, about 8.05 mg, about 8.055 mg, about 8.06 mg, about 8.065 mg, about 8.07 mg, about 8.075 mg, about 8.08 mg, about 8.085 mg, about 8.09 mg, about 8.095 mg, about 8.10 mg, about 8.105 mg, about 8.11 mg, about 8.115 mg, about 8.12 mg, about 8.125 mg, about 8.13 mg, about 8.135 mg, about 8.14 mg, about 8.145 mg, about 8.15 mg, about 8.155 mg, about 8.16 mg, about 8.165 mg, about 8.17 mg, about 8.175 mg, about 8.18 mg, about 8.185 mg, about 8.19 mg, about 8.195 mg, about 8.20 mg, about 8.205 mg, about 8.21 mg, about 8.215 mg, about 8.22 mg, about 8.225 mg, about 8.23 mg, about 8.235 mg, about 8.24 mg, about 8.245 mg, about 8.25 mg, about 8.255 mg, about 8.26 mg, about 8.265 mg, about 8.27 mg, about 8.275 mg, about 8.28 mg, about 8.285 mg, about 8.29 mg, about 8.295 mg, about 8.30 mg, about 8.305 mg, about 8.31 mg, about 8.315 mg, about 8.32 mg, about 8.325 mg, about 8.33 mg, about 8.335 mg, about 8.34 mg, about 8.345 mg, about 8.35 mg, about 8.355 mg, about 8.36 mg, about 8.365 mg, about 8.37 mg, about 8.375 mg, about 8.38 mg, about 8.385 mg, about 8.39 mg, about 8.395 mg, about 8.40 mg, about 8.405 mg, about 8.41 mg, about 8.415 mg, about 8.42 mg, about 8.425 mg, about 8.43 mg, about 8.435 mg, about 8.44 mg, about 8.445 mg, about 8.45 mg, about 8.455 mg, about 8.46 mg, about 8.465 mg, about 8.47 mg, about 8.475 mg, about 8.48 mg, about 8.485 mg, about 8.49 mg, about 8.495 mg, about 8.50 mg, about 8.505 mg, about 8.51 mg, about 8.515 mg, about 8.52 mg, about 8.525 mg, about 8.53 mg, about 8.535 mg, about 8.54 mg, about 8.545 mg, about 8.55 mg, about 8.555 mg, about 8.56 mg, about 8.565 mg, about 8.57 mg, about 8.575 mg, about 8.58 mg, about 8.585 mg, about 8.59 mg, about 8.595 mg, about 8.60 mg, about 8.605 mg, about 8.61 mg, about 8.615 mg, about 8.62 mg, about 8.625 mg, about 8.63 mg, about 8.635 mg, about 8.64 mg, about 8.645 mg, about 8.65 mg, about 8.655 mg, about 8.66 mg, about 8.665 mg, about 8.67 mg, about 8.675 mg, about 8.68 mg, about 8.685 mg, about 8.69 mg, about 8.695 mg, about 8.70 mg, about 8.705 mg, about 8.71 mg, about 8.715 mg, about 8.72 mg, about 8.725 mg, about 8.73 mg, about 8.735 mg, about 8.74 mg, about 8.745 mg, about 8.75 mg, about 8.755 mg, about 8.76 mg, about 8.765 mg, about 8.77 mg, about 8.775 mg, about 8.78 mg, about 8.785 mg, about 8.79 mg, about 8.795 mg, about 8.80 mg, about 8.805 mg, about 8.81 mg, about 8.815 mg, about 8.82 mg, about 8.825 mg, about 8.83 mg, about 8.835 mg, about 8.84 mg, about 8.845 mg, about 8.85 mg, about 8.855 mg, about 8.86 mg, about 8.865 mg, about 8.87 mg, about 8.875 mg, about 8.88 mg, about 8.885 mg, about 8.89 mg, about 8.895 mg, about 8.90 mg, about 8.905 mg, about 8.91 mg, about 8.915 mg, about 8.92 mg, about 8.925 mg, about 8.93 mg, about 8.935 mg, about 8.94 mg, about 8.945 mg, about 8.95 mg, about 8.955 mg, about 8.96 mg, about 8.965 mg, about 8.97 mg, about 8.975 mg, about 8.98 mg, about 8.985 mg, about 8.99 mg, or about 8.995 mg.

In some embodiments, the average dose of caryophyllene is about 9.00 mg, about, about 9.005 mg, about 9.01 mg, about 9.015 mg, about 9.02 mg, about 9.025 mg, about 9.03 mg, about 9.035 mg, about 9.04 mg, about 9.045 mg, about 9.05 mg, about 9.055 mg, about 9.06 mg, about 9.065 mg, about 9.07 mg, about 9.075 mg, about 9.08 mg, about 9.085 mg, about 9.09 mg, about 9.095 mg, about 9.10 mg, about 9.105 mg, about 9.11 mg, about 9.115 mg, about 9.12 mg, about 9.125 mg, about 9.13 mg, about 9.135 mg, about 9.14 mg, about 9.145 mg, about 9.15 mg, about 9.155 mg, about 9.16 mg, about 9.165 mg, about 9.17 mg, about 9.175 mg, about 9.18 mg, about 9.185 mg, about 9.19 mg, about 9.195 mg, about 9.20 mg, about 9.205 mg, about 9.21 mg, about 9.215 mg, about 9.22 mg, about 9.225 mg, about 9.23 mg, about 9.235 mg, about 9.24 mg, about 9.245 mg, about 9.25 mg, about 9.255 mg, about 9.26 mg, about 9.265 mg, about 9.27 mg, about 9.275 mg, about 9.28 mg, about 9.285 mg, about 9.29 mg, about 9.295 mg, about 9.30 mg, about 9.305 mg, about 9.31 mg, about 9.315 mg, about 9.32 mg, about 9.325 mg, about 9.33 mg, about 9.335 mg, about 9.34 mg, about 9.345 mg, about 9.35 mg, about 9.355 mg, about 9.36 mg, about 9.365 mg, about 9.37 mg, about 9.375 mg, about 9.38 mg, about 9.385 mg, about 9.39 mg, about 9.395 mg, about 9.40 mg, about 9.405 mg, about 9.41 mg, about 9.415 mg, about 9.42 mg, about 9.425 mg, about 9.43 mg, about 9.435 mg, about 9.44 mg, about 9.445 mg, about 9.45 mg, about 9.455 mg, about 9.46 mg, about 9.465 mg, about 9.47 mg, about 9.475 mg, about 9.48 mg, about 9.485 mg, about 9.49 mg, about 9.495 mg, about 9.50 mg, about 9.505 mg, about 9.51 mg, about 9.515 mg, about 9.52 mg, about 9.525 mg, about 9.53 mg, about 9.535 mg, about 9.54 mg, about 9.545 mg, about 9.55 mg, about 9.555 mg, about 9.56 mg, about 9.565 mg, about 9.57 mg, about 9.575 mg, about 9.58 mg, about 9.585 mg, about 9.59 mg, about 9.595 mg, about 9.60 mg, about 9.605 mg, about 9.61 mg, about 9.615 mg, about 9.62 mg, about 9.625 mg, about 9.63 mg, about 9.635 mg, about 9.64 mg, about 9.645 mg, about 9.65 mg, about 9.655 mg, about 9.66 mg, about 9.665 mg, about 9.67 mg, about 9.675 mg, about 9.68 mg, about 9.685 mg, about 9.69 mg, about 9.695 mg, about 9.70 mg, about 9.705 mg, about 9.71 mg, about 9.715 mg, about 9.72 mg, about 9.725 mg, about 9.73 mg, about 9.735 mg, about 9.74 mg, about 9.745 mg, about 9.75 mg, about 9.755 mg, about 9.76 mg, about 9.765 mg, about 9.77 mg, about 9.775 mg, about 9.78 mg, about 9.785 mg, about 9.79 mg, about 9.795 mg, about 9.80 mg, about 9.805 mg, about 9.81 mg, about 9.815 mg, about 9.82 mg, about 9.825 mg, about 9.83 mg, about 9.835 mg, about 9.84 mg, about 9.845 mg, about 9.85 mg, about 9.855 mg, about 9.86 mg, about 9.865 mg, about 9.87 mg, about 9.875 mg, about 9.88 mg, about 9.885 mg, about 9.89 mg, about 9.895 mg, about 9.90 mg, about 9.905 mg, about 9.91 mg, about 9.915 mg, about 9.92 mg, about 9.925 mg, about 9.93 mg, about 9.935 mg, about 9.94 mg, about 9.945 mg, about 9.95 mg, about 9.955 mg, about 9.96 mg, about 9.965 mg, about 9.97 mg, about 9.975 mg, about 9.98 mg, about 9.985 mg, about 9.99 mg, about 9.995 mg, or about 10 mg.

In some embodiments, the average dose of caryophyllene is about 10.0 mg, about 11.0 mg, about 12.0 mg, about 13.0 mg, about 14.0 mg, about 15.0 mg, about 16.0 mg, about 17.0 mg, about 18.0 mg, about 19.0 mg, about 20.0 mg, about 21.0 mg, about 22.0 mg, about 23.0 mg, about 24.0 mg, about 25.0 mg, about 26.0 mg, about 27.0 mg, about 28.0 mg, about 29.0 mg, about 30.0 mg, about 31.0 mg, about 32.0 mg, about 33.0 mg, about 34.0 mg, about 34.0 mg, about 36.0 mg, about 37.0 mg, about 38.0 mg, about 39.0 mg, about 40.0 mg, about 41.0 mg, about 42.0 mg, about 43.0 mg, about 44.0 mg, about 45.0 mg, about 46.0 mg, about 47.0 mg, about 48.0 mg, about 49.0 mg, about 50.0 mg, about 51.0 mg, about 52.0 mg, about 53.0 mg, about 54.0 mg, about 55.0 mg, about 56.0 mg, about 57.0 mg, about 58.0 mg, about 59.0 mg, about 60.0 mg, about 61.0 mg, about 62.0 mg, about 63.0 mg, about 64.0 mg, about 65.0 mg, about 66.0 mg, about 67.0 mg, about 68.0 mg, about 69.0 mg, about 70.0 mg, about 71.0 mg, about 72.0 mg, about 73.0 mg, about 74.0 mg, about 75.0 mg, about 76.0 mg, about 77.0 mg, about 78.0 mg, about 79.0 mg, about 80.0 mg, about 81.0 mg, about 82.0 mg, about 83.0 mg, about 84.0 mg, about 85.0 mg, about 86.0 mg, about 87.0 mg, about 88.0 mg, about 89.0 mg, about 90.0 mg, about 91.0 mg, about 92.0 mg, about 93.0 mg, about 94.0 mg, about 95.0 mg, about 96.0 mg, about 97.0 mg, about 98.0 mg, about 99.0 mg, or about 100 mg.

In some embodiments of the disclosure, the methods of treatment can include where the average dose of caryophyllene is administered to a subject in need thereof with a frequency from about once a week to about 100 times per day, for example once per week, 2 times per week, 3 times per week, 4 times per week, 5 times per week, 6 times per week, once a day, 2 times per day, 3 times per day, 4 times per day, 5 times per day, 6 times per day, 7 times per day, 8 times per day, 9 times per day, 10 times per day, 11 time per day, 12 times per day, 13 times per day, 14 times per day, 15 times per day, 16 times per day, 17 times per day, 18 times per day, 19 times per day, 20 times per day, 21 times per day, 22 times per day, 23 times per day, 24 times per day, 25 times per day, 26 times per day, 27 times per day, 28 times per day, 29 times per day, 30 times per day, 31 times per day, 32 times per day, 33 times per day, 34 times per day, 35 times per day, 36 times per day, 37 times per day, 38 times per day, 39 times per day, 40 times per day, 41 times per day, 42 times per day, 43 times per day, 44 times per day, 45 times per day, 46 times per day, 47 times per day, 48 times per day, 49 times per day, 50 times per day, 55 times per day, 60 times per day, 70 times per day, 80 times per day, 90 times per day, or 100 times per day.

In some embodiments, the inhaled caryophyllene can be administered at or near the same time as another agent is administered to a patient or user such that the inhaled caryophyllene has a synergistic effect with the other agent. For example, the inhaled caryophyllene can be administered at the same time or shortly after (e.g., 1 min, 5 min, 10 min, 15 min, 20 min, 30 min, etc.) a patient has taken a dose of another medicine (such as a medicine for pain) via another administration method or route (e.g., orally (liquid or capsule), injected, and/or the like).

Caryophyllene compositions of the disclosure are pure, essentially pure, or substantially pure. The caryophyllene compositions can be of about 50% purity, about 55% purity, about 60% purity, about 65% purity, about 70% purity, about 75% purity, about 80% purity, about 85% purity, about 90% purity, about 95% purity, about 99% purity, or greater than about 99% purity.

In some embodiments, additional components are optionally added to the caryophyllene composition to improve the taste and/or physical properties of the composition (such as stability, viscosity, appearance of smoke as it is exhaled, etc.). Such additional components include, but are not limited to, sweeteners, natural flavorants, artificial flavorants, colorants, antioxidants, preservatives, chelating agents, viscomodulators, tonicifiers, odorants, opacifiers, suspending agents, binders, thickeners, carriers and mixtures thereof, including, but not limited to, xanthum gum, carboxymethylcellulose, carboxyethylcellulose, hydroxypropylcellulose, methylcellulose, microcrystalline cellulose, starches, dextrins, maltodextrins, other polyols (including sugar alcohols, such as sorbitol, lactitol or mannitol), carbohydrates (e.g., lactose), propylene glycol alginate, gellan gum, guar, pectin, tragacanth gum, gum acacia, locust bean gum, gum arabic, mannitol, sucralose, silicon dioxide, stearic acid, hydroxypropyl methylcellulose, mono-, di- and triglycerides (acyl glycerols), ether and sugar acetates or other acid esters such as dimethyl acetate, ethyl acetate, isopropyl acetate, ethylhexyl acetate, butyl acetate, triethyl citrate, dimethyl butyrate and the like.

In some embodiments, the carrier comprises one or more medium chain length triglycerides (MCTs). MCTs are triglycerides whose fatty acids have an aliphatic tail of 6-12 carbon atoms. In certain embodiments, the MCT is one or more of caproic acid, caprylic acid, capric acid, lauric acid and mixtures thereof. Suitable sources of MCTs are known to those skilled in the art and include, for example, coconut oil and palm kernel oil.

In some embodiments, the carrier comprises one or more polyesterdiols. The polyesterdiol may be a linear two to ten units polymer (also referred to as (ester)₂₋₁₀ glycol), that is derived from natural or non-natural sources such as vegetables, fruits, bacteria, yeast, algae, or manufactured by chemical processes.

For example, in some embodiments, the polyesterdiol is 1) a polypropylene glycol such as: dipropylene glycol; tripropylene glycol, including tetra-, penta-, hexa-, hepta-, octa-, nona- and decapropylene glycol and other derivatives thereof; 2) a polybutylene glycol such as: dibutylene glycol, tributylene glycol, including tetra-, penta-, hexa-, hepta-, octa-, nona- and decabutylene glycol, and other derivatives thereof; 3) also including 2-10 unit polymers of rare organic ester types such as pentylene, octylene, terpentylene, nonylene, linalylene, isoamylene, isobutylene, geranylene, bornylene, benzylene and allylene, caprylylene, such as, for example, polyisobutylene glycol such as diisobutylene glycol; and 4) triethylene glycol, including tetra-, penta-, hexa-, hepta-, octa-, nona- and decaethylene glycols and other derivatives thereof such as acid or sugar conjugates, and esters or ether or alcohol derivatives.

In some embodiments, the carrier is a linear polyesterdiol selected from the group consisting of: (ethylene)₃₋₁₀ glycol; (propylene)₂₋₁₀ glycol; (butylene)₂₋₁₀ glycol; (pentylene)₂₋₁₀ glycol; (octylene)₂₋₁₀ glycol; (terpentylene)₂₋₁₀ glycol; (nonylene)₂₋₁₀ glycol; (linalylene)₂₋₁₀ glycol; (isoamylene)₂₋₁₀ glycol; (isobutylene)₂₋₁₀ glycol; (geranylene)₂₋₁₀ glycol; (bornylene)₂₋₁₀ glycol; (benzylene)₂₋₁₀ glycol; (allylene)₂₋₁₀ glycol; and (caprylylene)₂₋₁₀ glycol; acid or sugar conjugates thereof, and ester or ether or alcohol derivatives thereof.

In some embodiments, the carrier is selected from the group consisting of: triethylene glycol; tetraethyleneglycol, pentaethylenglycol, hexaethyleneglycol, heptaethyleneglycol, octaethyleneglycol, nonaethylenglycol; decaethylene glycol; dipropylene glycol; tripropylene glycol; tetrapropylene glycol, pentapropylene glycol, hexapropylene glycol, heptapropylene glycol, octapropylene glycol, nonapropylene glycol; decapropylene glycol; dibutylene glycol, tributylene glycol; tetrabutylene glycol, pentabutylene glycol, hexabutylene glycol, heptabutylene glycol, octabutylene glycol, nonabutylene glycol, decabutylene glycol and diisobutylene glycol.

In some embodiments, the carrier is selected from the group consisting borneol, camphor, 1,8-Cineole, citral, geraniol, indomethacin, limonene, linalool, linalyl acetate, β-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone

In some embodiments, the caryophyllene composition comprises one or more of propylene glycol, glycerine, vegetable glycerine, and/or water.

If desired, one or more flavorants may be added to the composition. Non-limiting examples include wintergreen, propolis, eucalyptus, tobacco, Turkish tobacco, menthol, peppermint, spearmint, green tea, cream, natural sweetener, banana, strawberry, blueberry, mango, raspberry, peach, pineapple, apple, orange, papaya, watermelon, grape, cherry, coffee, chocolate, vanilla, cola, piña colada, coconut, almond, cinnamon, white chocolate, rum, butterscotch, and caramel or the like. The total amount of flavorants typically ranges from about 0.5 to about 15 wt. %, such as from about 1 to about 10 wt. %, based on the total weight of the composition.

Caryophyllene compositions of the disclosure can be free, essentially free, or substantially free from one or more other terpenes, such as free, essentially free, or substantially free from one or more of: terpinolene, alpha phelladrene, beta ocimene, carene, limonene, gamma terpinene, alpha pinene, alpha terpinene, beta pinene, fenchol, camphene, alpha terpineol, linalool, caryphyllene oxide, and/or myrcene.

Caryophyllene compositions of the disclosure can be free, essentially free, or substantially free from one or more other cannabinoids, such as free, essentially free, or substantially free from one or more of delta-9-tetrahydrocannabinol (THC) and/or cannabidiol (CBD).

Caryophyllene compositions of the disclosure can be free, essentially free, or substantially free from one or more carriers. For example, carriers that are not present in the caryophyllene compositions can include borneol, camphor, 1,8-Cineole, citral, geraniol, indomethacin, limonene, linalool, linalyl acetate, β-myrcene, myrcenol, 1-menthol, menthone, neomenthol, nerol, nerolidol, a-pinene, peppermint oil, pulegone, phytol, terpineol, terpinen-4-ol, thymohydroquinone, thymol, and thymoquinone.

In some embodiments, the caryophyllene composition is administered via a handheld electronic device (caryophyllene device) that vaporizes the caryophyllene for inhalation by a user. In some embodiments, the caryophyllene composition and/or caryophyllene device do not include or contain additional constituents, carriers, or additives. For example, the caryophyllene composition can be pure, essentially pure, or substantially pure, for example, having about 50% purity, about 55% purity, about 60% purity, about 65% purity, about 70% purity, about 75% purity, about 80% purity, about 85% purity, about 90% purity, about 95% purity, about 99% purity, or having greater than about 99% purity.

In some embodiments, the caryophyllene composition is free from, essentially free from, or substantially free from one or more of: a carrier, propylene glycol, glycerine, vegetable glycerine, water, and/or flavorants. In some embodiments, the caryophyllene composition is free from, essentially free from, or substantially free from one or more other terpenes and one or more other cannabinoids (i.e., only BCP and ACP). It is to be understood that the percentages and purities discussed herein can be in weight or volume, depending on the embodiment and/or implementation.

In some embodiments, components of an caryophyllene device include a mouthpiece, an caryophyllene reservoir (which can be a tank or cartridge), a heating element or other atomizer (e.g., piezoelectric atomizer, inkjet-type atomizer, etc.), and an actuator (such as a switch or pressure sensor) to activate the heating element/atomizer. Depending on the embodiment, the caryophyllene reservoir can be configured to store between about 0.5 mg and 500 mg of a caryophyllene composition/caryophyllene liquid, for example, between about 1 mg and about 250 mg, about 2 mg and 150 mg, about 3 mg and about 100 mg, about 4 mg and about 75 mg, about 5 mg and about 50 mg, or between about 10 mg and about 30 mg.

Depending on the embodiment, the caryophyllene reservoir can be configured to store about 0.1 ml, about 0.5 ml, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, about 50 ml, about 55 ml, about 60 ml, about 70 ml, about 80 ml, about 90 ml, about 100 ml, or more than 100 ml.

Some implementations of the caryophyllene device can include a microprocessor and a battery or other power source. Some implementations include and/or define a caryophyllene vapor chamber or caryophyllene atomization chamber. In some embodiments, an atomizer can include a small heater (e.g., heat coil or other heat element), that vaporizes a liquid caryophyllene composition stored in the caryophyllene reservoir. Some embodiments include a one or more wicks or wicking material configured to draw liquid caryophyllene composition onto or into the heater or atomizer. In some embodiments, when a user activates an actuator (i.e., activates a pressure sensor by inhaling, presses a switch), the atomizer is activated (e.g., the heater is powered) and the liquid caryophyllene composition is atomized or vaporized.

Depending on the embodiment and/or implementation, the heater and/or liquid caryophyllene composition can reach a temperature from about 75° C. to about 350° C., from about 80° C. to about 300° C., from about 85° C. to about 275° C., from about 90° C. to about 250° C., from about 95° C. to about 225° C., from about 100° C. to about 200° C., from about 101° C. to about 175° C., from about 102° C. to about 150° C., from about 103° C. to about 125° C., from about 104° C. to about 115° C., or from about 105° C. to about 110° C. Some embodiments can include devices and/or configurations such as discussed in one or more of the following, including combinations thereof: U.S. Pat. App. Pub. No. 2017/0023235; U.S. Pat. App. Pub. No. 2015/0114411; U.S. Pat. App. Pub. No. 2016/0000149; U.S. Pat. App. Pub. No. 2016/0270451; U.S. Pat. App. Pub. No. 2016/0053988; U.S. Pat. App. Pub. No. 2015/0327595; U.S. Pat. App. Pub. No. 2014/0299125; U.S. Pat. App. Pub. No. 2014/0182608; U.S. Pat. App. Pub. No. 2014/0286630; WIPO Patent Application Publication WO/2016/156497; WIPO Patent Application Publication WO/2016/030369; U.S. Pat. Nos. 9,320,300; 9,095,175; 9,259,035; 9,326,549; 7,997,280; 9,459,021; 9,427,711; and/or U.S. Pat. No. 9,352,288; each of which is expressly incorporated by reference herein for all purposes.

In some embodiments, the atomizer of a caryophyllene device of the disclosure is configured to vaporize an average dose per actuation such that an average dose can be inhaled by a single inhalation of a user.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the disclosed teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though discussed as sequential acts in illustrative embodiments.

This application may contain material that is subject to copyright, mask work, and/or other intellectual property protection. The respective owners of such intellectual property have no objection to the facsimile reproduction of the disclosure by anyone as it appears in published Patent Office file/records, but otherwise reserve all rights.

The following non-limiting examples illustrate various aspects of the present invention.

### EXAMPLES

### Example 1: Preparation of BCP Compositions

Examples 1-3 utilized the following materials: low purity (>80%) beta-caryophyllene and MCT derived from coconut.

BCP compositions having the following concentrations were prepared: 1.0 weight percent (wt %) BCP; 2.0 wt % BCP; 5.0 wt % BCP; 10.0 wt % BCP and 50.0 wt % BCP.

The compositions were prepared in pre-cleaned glass beakers and magnetic stir bars that had been dried in an oven at about 200° F. for 20 minutes.

1 wt % BCP: 15.57 g of MCT was added to a beaker and then 0.159 g BCP was added to the MCT-containing beaker. A stir bar was added and the solution was stirred for at least 1 min.

The 2.0 wt % BCP; 5.0 wt % BCP; 10.0 wt % BCP and 50.0 wt % BCP compositions were prepared using the procedure described above using the amount of BCP and MCT shown in the following Table 1. The 0.0% BCP was a negative control used in the Psychoactive Effect Study of Example 3.

TABLE 1
Concentrations of BCP

| wt % BCP | Mass of MCT (g) | Mass of BCP (g) |
|---|---|---|
| 0.0% | 20 | 0 |
| 1.0% | 15.57 | 0.159 |
| 2.0% | 18.733 | 0.376 |
| 5.0% | 16.168 | 0.854 |
| 10.0% | 16.387 | 1.826 |
| 50.0% | 11.654 | 11.664 |

The BCP compositions were tested for terpene content using a gas chromatograph equipped with a flame ionization detector (FID). The results are shown in the following Table 2.

TABLE 2
Concentrations of BCP and ACP

| wt % BCP | β-caryophyllene | α-humulene | Total |
|---|---|---|---|
| 0.0% | 0.00% | 0.00% | 0.00% |
| 1.0% | 0.96% | 0.07% | 1.03% |
| 2.0% | 1.87% | 0.13% | 2.00% |
| 5.0% | 4.73% | 0.33% | 5.06% |
| 10.0% | 9.71% | 0.69% | 10.39% |
| 50.0% | 48.17% | 3.38% | 51.55% |

### Example 2: Preparation of BCP-Containing Apparatus

The BCP compositions of Example 1 were used to prepare BCP-containing Suniva disposeable ‘e-pens’. 120 e-pens were prepared (20 for each of the six BCP concentrations) according to the following procedure.

For the 0 wt % BCP concentration, ˜150 μL MCT was pipetted into 20 e-pens. The septa and cap were then secured at the top of the pen.

For the 1.0 wt %, 2.0 wt %, 5.0 wt %, 10.0 wt % and 50.0 wt % BCP concentrations, while the solution is being stirred, 150 μL of the solution was transferred into 20 e-pens. Septa and cap were then secured at the top of the pen.

### Example 3: Psychoactive Effects of BCP

The primary objective was to evaluate the psychoactive effects of the BCP compositions of the present disclosure. Volunteers were provided unlabeled e-pens prepared in Example 2 (i.e., BCP concentrations: 0.0 wt %, 1.0 wt %, 2.0 wt %, 5.0 wt %, 10.0 wt % and 50.0 wt %).

Participants

Thirteen volunteers participated in the study. Volunteers provided the following background information: (1) year of birth, (2) gender (Male or Female), (3) cannabis use (Regularly, Sometimes, Infrequently or Never) and tobacco/cigarette use (Regularly, Sometimes, Infrequently or Never).

Product Feedback Survey

Psychoactive effects of the BCP-containing compositions were measured using a Product Feedback Survey. Volunteers were asked to test the product by keeping the pen vertical with the metal end facing down, and inhaling from the mouthpiece for 3-5 seconds. Volunteers were asked to do this three times per product, and wait 30 minutes to complete a survey. Volunteers were limited to one trial per day, in order to avoid the effects of one trial from bleeding into the next.

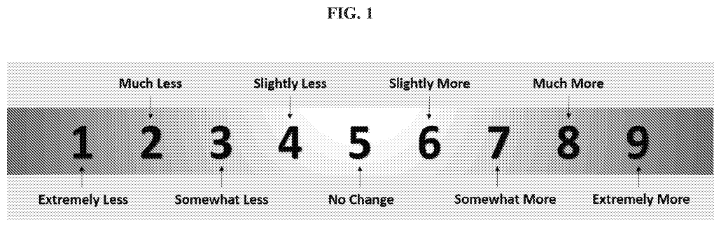

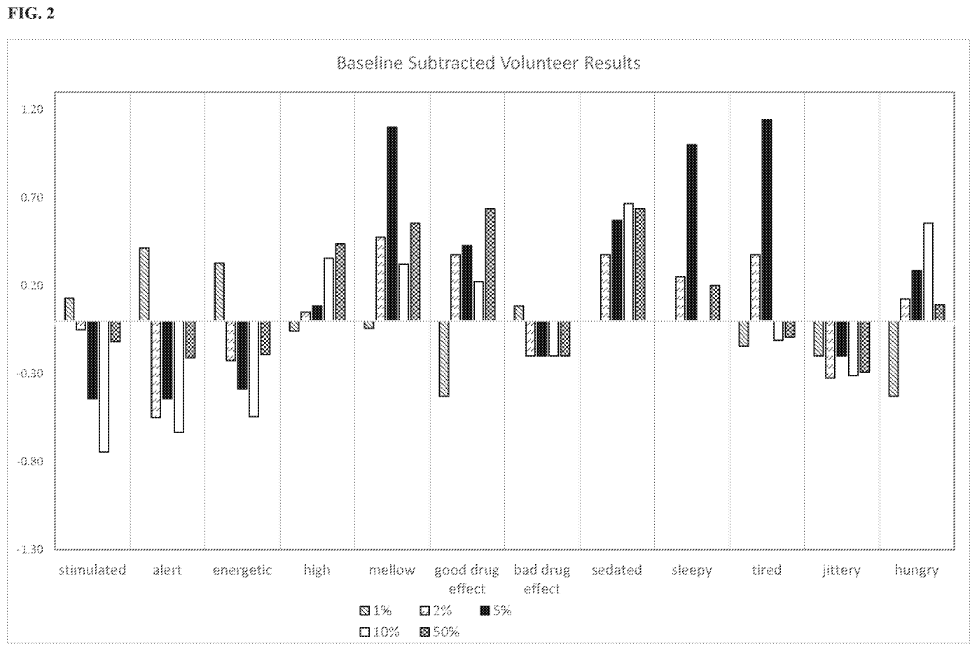

Volunteers completed the survey using a Numerical Rating Scale (NRS) that measured the volunteer's reported change from baseline for a series of emotions, physical characteristics and desires. The range of the NRS was 0-9 with anchors at 0 “Extremely Less”; 5 “No Change”; and 10 “Extremely More”. The NRS is shown at FIG. 1.

Volunteers were asked to circle a number using the NRS to complete a sentence from 3 subcategories: (1) “Right now, I feel . . . .”, (2) “Right now I have . . . .” and (3) “Right now my desire for the following is . . . .” The following Table 3 shows the categories of the survey. Each category required a score of 0-9 as described above.

TABLE 3
Vaporizable composition survey
Circle a number using the provided scale to complete the sentence
“Right now, I feel . . .”

| | | | | |
|---|---|---|---|---|
| stimulated | alert | energetic | high | mellow |
| tired | jittery | restless | hungry | nauseous |
| friendly | self-confident | social | miserable | withdrawn |
| confused | dizzy | forgetful | like my heart is | like noises or |
| | | | beating faster | sounds seem |
| | | | than usual | louder than usual |
| good drug effect | bad drug effect | clumsy | sedated | sleepy |
| muscle pain | content | depressed | irritable | talkative |
| unmotivated | angry | suspicious | anxious | on edge |
| motivated | focused | hungry | relaxed | |

Circle a number using the provided scale to complete the sentence
“Right now, I have . . .”

| | | | | |
|---|---|---|---|---|
| blurred vision | an upset stomach | stomach pain | a headache | difficulty |
| | | | | concentrating |

| | |
|---|---|
| chills | a desire to and use this product again |

Circle a number using the provided scale to complete the sentence
“Right now my desire for the following is . . .”

| | | | | |
|---|---|---|---|---|
| alcohol | food | stomach pain | cannabis | tobacco/cigarette |

Results

The volunteers completed the Survey 30 minutes after inhalation. The results for each volunteer was recorded. The results for all volunteers were averaged for each BCP concentration.

The average results are shown in the following three Tables 4-6:

TABLE 4
Average scores for first part of survey

| | BCP concentration | | | | | |
|---|---|---|---|---|---|---|
| | 0 wt % | 1 wt % | 2 wt % | 5 wt % | 10 wt % | 50 wt % |
| stimulated | 5.3 | 5.4 | 5.3 | 4.9 | 4.6 | 5.2 |
| alert | 5.3 | 5.7 | 4.8 | 4.9 | 4.7 | 5.1 |
| energetic | 5.1 | 5.4 | 4.9 | 4.7 | 4.6 | 4.9 |
| high | 5.2 | 5.1 | 5.3 | 5.3 | 5.6 | 5.6 |
| mellow | 4.9 | 4.9 | 5.4 | 6.0 | 5.2 | 5.5 |
| good drug | 5.0 | 4.6 | 5.4 | 5.4 | 5.2 | 5.6 |
| effect | | | | | | |
| bad drug | 5.2 | 5.3 | 5.0 | 5.0 | 5.0 | 5.0 |
| effect | | | | | | |
| clumsy | 5.0 | 4.9 | 4.9 | 5.1 | 4.9 | 5.2 |
| sedated | 5.0 | 5.0 | 5.4 | 5.6 | 5.7 | 5.6 |
| sleepy | 5.0 | 5.0 | 5.3 | 6.0 | 5.0 | 5.2 |
| tired | 5.0 | 4.9 | 5.4 | 6.1 | 4.9 | 4.9 |
| jittery | 5.2 | 5.0 | 4.9 | 5.0 | 4.9 | 4.9 |
| restless | 5.1 | 5.0 | 5.0 | 5.0 | 4.9 | 5.0 |
| hungry | 5.0 | 4.6 | 5.1 | 5.3 | 5.6 | 5.1 |
| nauseous | 5.0 | 4.9 | 5.0 | 5.0 | 5.0 | 4.9 |
| muscle pain | 5.0 | 4.9 | 5.0 | 4.4 | 4.8 | 5.3 |
| content | 5.0 | 5.4 | 5.1 | 5.4 | 5.3 | 5.2 |
| depressed | 5.1 | 5.0 | 4.9 | 5.0 | 5.0 | 4.9 |
| irritable | 5.1 | 5.1 | 5.0 | 5.0 | 4.6 | 4.8 |
| talkative | 5.0 | 4.7 | 5.0 | 5.1 | 5.0 | 4.5 |
| friendly | 5.0 | 4.9 | 4.9 | 5.0 | 5.2 | 5.4 |
| self-confident | 5.0 | 5.1 | 5.0 | 5.1 | 5.1 | 4.9 |
| social | 5.1 | 4.7 | 4.8 | 5.7 | 5.0 | 5.0 |
| miserable | 5.0 | 4.7 | 5.0 | 5.0 | 4.7 | 5.0 |
| withdrawn | 5.0 | 4.7 | 5.3 | 5.1 | 5.2 | 5.2 |
| unmotivated | 5.1 | 4.9 | 5.5 | 5.6 | 4.8 | 4.5 |
| angry | 5.1 | 4.7 | 5.0 | 5.0 | 4.8 | 4.7 |
| suspicious | 5.0 | 5.1 | 5.1 | 5.0 | 4.8 | 4.9 |
| anxious | 5.1 | 5.1 | 5.1 | 5.1 | 5.1 | 5.1 |
| on edge | 5.1 | 5.1 | 5.0 | 5.3 | 5.2 | 5.1 |
| confused | 5.0 | 5.3 | 5.1 | 5.1 | 5.0 | 5.2 |
| dizzy | 5.1 | 5.3 | 5.3 | 5.0 | 5.1 | 5.2 |
| forgetful | 5.0 | 4.9 | 5.1 | 5.3 | 5.3 | 4.9 |
| like my heart is | 5.1 | 4.7 | 5.3 | 5.0 | 5.0 | 5.1 |
| beating faster | | | | | | |
| than usual | | | | | | |
| like noises or | 5.0 | 5.1 | 5.1 | 5.0 | 5.2 | 5.3 |
| sounds seem | | | | | | |
| louder than | | | | | | |
| usual | | | | | | |
| motivated | 5.0 | 5.1 | 4.8 | 5.1 | 4.7 | 5.1 |
| focused | 5.0 | 4.9 | 4.8 | 5.1 | 4.7 | 5.1 |
| hungry | 5.0 | 4.6 | 5.1 | 5.4 | 5.3 | 5.4 |
| relaxed | 5.0 | 5.0 | 5.1 | 5.6 | 5.3 | 5.4 |

TABLE 5
Average scores for second part of survey

| | BCP concentration | | | | | |
|---|---|---|---|---|---|---|
| | 0 wt % | 1 wt % | 2 wt % | 5 wt % | 10 wt % | 50 wt % |
| blurred vision | 5.0 | 5.0 | 5.3 | 5.1 | 5.0 | 4.9 |
| an upset | 5.0 | 5.0 | 5.0 | 5.0 | 5.0 | 5.1 |
| stomach | | | | | | |
| stomach pain | 5.1 | 5.0 | 5.0 | 5.0 | 5.0 | 5.0 |
| a headache | 5.1 | 5.0 | 4.9 | 5.0 | 4.9 | 5.0 |
| difficulty | 5.1 | 5.1 | 5.4 | 5.0 | 5.2 | 5.2 |
| concentrating | | | | | | |
| chills | 5.0 | 4.6 | 5.0 | 5.0 | 5.0 | 4.6 |
| a desire to | 4.8 | 3.6 | 5.0 | 3.8 | 4.2 | 4.8 |
| use this | | | | | | |
| product again | | | | | | |

TABLE 6
Average scores for third part of survey

| | BCP concentration | | | | | |
|---|---|---|---|---|---|---|
| | 0 wt % | 1 wt % | 2 wt % | 5 wt % | 10 wt % | 50 wt % |
| alcohol | 5.0 | 5.1 | 4.3 | 5.3 | 5.6 | 5.1 |
| food | 5.0 | 4.7 | 4.9 | 5.0 | 5.8 | 5.2 |
| cannabis | 5.3 | 6.2 | 5.4 | 5.8 | 6.0 | 5.9 |
| tobacco/ | 5.4 | 5.2 | 4.6 | 4.8 | 6.3 | 5.2 |
| cigarette | | | | | | |

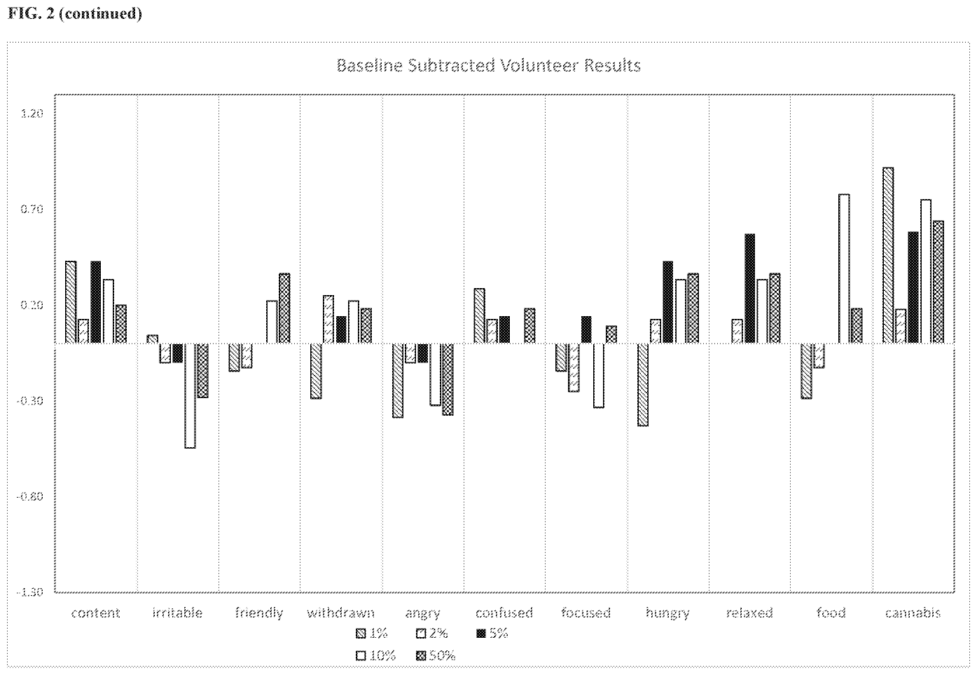

The baseline subtracted Survey results were calculated to determine the psychoactive effects of the BCP compositions. The average Survey results reported for the 0 wt % BCP group were used as the baseline (i.e., control group). The baseline subtracted Survey results are shown in the following Table 7 and a summary of the results are graphically represented in FIG. 2.

TABLE 7
Survey scores normalized with baseline
subtracted from trial averages

| Percent | | | | | | good drug |
|---|---|---|---|---|---|---|
| wt % | stimulated | alert | energetic | high | mellow | effect |
| 1 | 0.13 | 0.41 | 0.33 | −0.06 | −0.04 | −0.43 |
| 2 | −0.05 | −0.55 | −0.23 | 0.05 | 0.48 | 0.38 |
| 5 | −0.44 | −0.44 | −0.39 | 0.09 | 1.10 | 0.43 |
| 10 | −0.74 | −0.63 | −0.54 | 0.36 | 0.32 | 0.22 |
| 50 | −0.12 | −0.21 | −0.19 | 0.44 | 0.55 | 0.64 |

| Percent | bad drug | | | | | |
|---|---|---|---|---|---|---|
| wt % | effect | clumsy | sedated | sleepy | tired | jittery |
| 1 | 0.09 | −0.14 | 0.00 | 0.00 | −0.14 | −0.20 |
| 2 | −0.20 | −0.13 | 0.38 | 0.25 | 0.38 | −0.33 |
| 5 | −0.20 | 0.14 | 0.57 | 1.00 | 1.14 | −0.20 |
| 10 | −0.20 | −0.11 | 0.67 | 0.00 | −0.11 | −0.31 |
| 50 | −0.20 | 0.18 | 0.64 | 0.20 | −0.09 | −0.29 |

| Percent | | | | muscle | | |
|---|---|---|---|---|---|---|
| wt % | restless | hungry | nauseous | pain | content | depressed |
| 1 | −0.10 | −0.43 | −0.14 | −0.14 | 0.43 | −0.10 |
| 2 | −0.10 | 0.13 | 0.00 | 0.00 | 0.13 | −0.23 |
| 5 | −0.10 | 0.29 | 0.00 | −0.57 | 0.43 | −0.10 |
| 10 | −0.21 | 0.56 | 0.00 | −0.22 | 0.33 | −0.10 |
| 50 | −0.10 | 0.09 | −0.09 | 0.27 | 0.20 | −0.19 |

| Percent | | | | self- | | |
|---|---|---|---|---|---|---|
| wt % | irritable | talkative | friendly | confident | social | miserable |
| 1 | 0.04 | −0.29 | −0.14 | 0.14 | −0.39 | −0.29 |
| 2 | −0.10 | 0.00 | −0.13 | 0.00 | −0.35 | 0.00 |
| 5 | −0.10 | 0.14 | 0.00 | 0.14 | 0.61 | 0.00 |
| 10 | −0.54 | 0.00 | 0.22 | 0.11 | −0.10 | −0.33 |
| 50 | −0.28 | −0.55 | 0.36 | −0.09 | −0.10 | 0.00 |

| Percent | | | | | | |
|---|---|---|---|---|---|---|
| wt % | withdrawn | unmotivated | angry | suspicious | anxious | on edge |
| 1 | −0.29 | −0.24 | −0.39 | 0.14 | 0.04 | 0.04 |
| 2 | 0.25 | 0.40 | −0.10 | 0.13 | 0.03 | −0.10 |
| 5 | 0.14 | 0.47 | −0.10 | 0.00 | 0.04 | 0.19 |
| 10 | 0.22 | −0.32 | −0.32 | −0.22 | 0.01 | 0.12 |
| 50 | 0.18 | −0.65 | −0.37 | −0.10 | −0.01 | −0.01 |

| | | | | like my | like noises | |
|---|---|---|---|---|---|---|
| | | | | heart is | or sounds | |
| Percent | con- | | for- | beating faster | seem louder | moti- |
| wt % | fused | dizzy | getful | than usual | than usual | vated |
| 1 | 0.29 | 0.19 | −0.14 | −0.39 | 0.14 | 0.14 |
| 2 | 0.13 | 0.15 | 0.13 | 0.15 | 0.13 | −0.25 |
| 5 | 0.14 | −0.10 | 0.29 | −0.10 | 0.00 | 0.14 |
| 10 | 0.00 | 0.01 | 0.33 | −0.10 | 0.22 | −0.33 |
| 50 | 0.18 | 0.08 | −0.09 | 0.00 | 0.27 | 0.09 |

| Percent | | | | blurred | an upset | stomach |
|---|---|---|---|---|---|---|
| wt % | focused | hungry | relaxed | vision | stomach | pain |
| 1 | −0.14 | −0.43 | 0.00 | 0.00 | 0.00 | −0.10 |
| 2 | −0.25 | 0.13 | 0.13 | 0.25 | 0.00 | −0.10 |
| 5 | 0.14 | 0.43 | 0.57 | 0.14 | 0.00 | −0.10 |
| 10 | −0.33 | 0.33 | 0.33 | 0.00 | 0.00 | −0.10 |
| 50 | 0.09 | 0.36 | 0.36 | −0.09 | 0.09 | −0.10 |

| | a | | | a desire to | | |
|---|---|---|---|---|---|---|
| Percent | head- | difficulty | | use this | alco- | |
| wt % | ache | concentrating | chills | product again | hol | food |
| 1 | −0.10 | 0.04 | −0.43 | −1.23 | 0.14 | −0.29 |
| 2 | −0.23 | 0.28 | 0.00 | 0.20 | −0.75 | −0.13 |
| 5 | −0.10 | −0.10 | 0.00 | −0.97 | 0.29 | 0.00 |
| 10 | −0.21 | 0.12 | 0.00 | −0.58 | 0.56 | 0.78 |
| 50 | −0.10 | 0.08 | −0.36 | 0.02 | 0.09 | 0.18 |

| Percent | | |
|---|---|---|
| wt % | cannabis | tobacco/cigarette |
| 1 | 0.92 | −0.20 |
| 2 | 0.18 | −0.80 |
| 5 | 0.58 | −0.65 |
| 10 | 0.75 | 0.85 |
| 50 | 0.64 | −0.23 |

The results indicate that the caryophyllene compositions of the present disclosure may be useful in the treatment of pain, depression, sleep disorders and hyperactivity disorders.

### Example 4

A human trial (n=3) was conducted in which subjects with varying level of cannabis exposure (a subject that was a regular user, a subject that was an occasional user, and a cannabis-naïve subject) were each administered a BCP/ACP composition as described above, having a ratio of BCP/ACP between about 11:1 and 8.8:1, and being free or essentially free from THC and CBD. Using a vape pen, each subject took a puff lasting 2-seconds or less, with an estimated intake of 2 mg or less of the BCP composition (for the cannabis-naïve subject) to about 5 mg of the BCP composition. Surprisingly, each subject noted psychoactive effects from the single dose, including sensations of increased energy. In a separate trial (n=2) with different subjects and the same compositions, both subjects observed a calming effect from doses of about 2-5 mg of the vaporized caryophyllene composition administered via inhalation.

### Example 5

The experiments outlined in Examples 1-3 will be repeated using pure (>99%) BCP as described below. BCP compositions having the following concentrations will be prepared: 1.0 weight percent (wt %) BCP; 2.0 wt % BCP; 5.0 wt % BCP; 10.0 wt % BCP and 50.0 wt % BCP. Additional BCP concentrations may also be produced and tested, including 20 wt %, 30 wt. %, and 40 wt. %.

The BCP compositions will be tested for terpene content using a gas chromatograph equipped with a flame ionization detector (FID) to ensure adherence to the planned formulations. The expected formulations are provided below in the following Table 8.

TABLE 8
Concentrations of BCP

| wt % BCP | β-caryophyllene | α-humulene | Total |
|---|---|---|---|
| 0.0% | 0.0% | 0.00% | 0.0% |
| 1.0% | 1.0% | 0.00% | 1.0% |
| 2.0% | 2.0% | 0.00% | 2.0% |
| 5.0% | 5.0% | 0.00% | 5.0% |
| 10.0% | 10.0% | 0.00% | 10.0% |
| 20.0% | 20.0% | 0.00% | 20.0% |
| 30.0% | 30.0% | 0.00% | 30.0% |
| 40.0% | 40.0% | 0.00% | 40.0% |
| 50.0% | 50.0% | 0.00% | 50.0% |

The BCP compositions of Table 8 will be used to prepare BCP-containing disposable ‘e-pens’. Between 60-120 e-pens will be prepared (10-20 for each of the tested BCP concentrations) according to the following procedure. Pens will be labeled according to a number or letter code so that participants are not able to glean the formulation from the label.

Volunteers will be provided mask-labeled e-pens prepared in this Example for testing. At least 10 volunteers will be asked to participate by testing each.

### Product Feedback Survey

Psychoactive effects of the BCP-containing compositions will be measured using a Product Feedback Survey. Volunteers will be asked to test the product by keeping the pen vertical with the metal end facing down, and inhaling from the mouth piece for 3-5 seconds. Volunteers will be asked to do this three times per product, and wait 30 minutes to complete a survey. Volunteers will be limited to one trial per day, in order to avoid the effects of one trial from bleeding into the next.

Volunteers will complete the survey described in Examples 1-3. Results for this survey will be presented as 1) average scores for each category, and 2) baseline subtracted scores to tease out the specific effects of each BCP formulation. Results will also be presented as a graph similar to that of FIG. 2.

### Example 6

The experiments outlined in Examples 1-3 will be repeated using pure (>99%) ACP as described below. ACP compositions having the following concentrations will be prepared: 1.0 weight percent (wt %) ACP; 2.0 wt % ACP; 5.0 wt % ACP; 10.0 wt % ACP and 50.0 wt % ACP. Additional ACP concentrations may also be produced and tested, including 20 wt %, 30 wt. %, and 40 wt. %.

The ACP compositions will be tested for terpene content using a gas chromatograph equipped with a flame ionization detector (FID) to ensure adherence to the planned formulations. The expected formulations are provided below in the following Table 9.

TABLE 9
Concentrations of ACP

| wt % ACP | β-caryophyllene | α-humulene | Total |
|---|---|---|---|
| 0.0% | 0.0% | 0.0% | 0.0% |
| 1.0% | 0.0% | 1.0% | 1.0% |
| 2.0% | 0.0% | 2.0% | 2.0% |
| 5.0% | 0.0% | 5.0% | 5.0% |
| 10.0% | 0.0% | 10.0% | 10.0% |
| 20.0% | 0.0% | 20.0% | 20.0% |
| 30.0% | 0.0% | 30.0% | 30.0% |
| 40.0% | 0.0% | 40.0% | 40.0% |
| 50.0% | 0.0% | 50.0% | 50.0% |

The ACP compositions of Table 9 will be used to prepare ACP-containing disposable ‘e-pens’. Between 60-120 e-pens will be prepared (10-20 for each of the tested ACP concentrations) according to the following procedure. Pens will be labeled according to a number or letter code so that participants are not able to glean the formulation from the label.

Volunteers will be provided mask-labeled e-pens prepared in this Example for testing. At least 10 volunteers will be asked to participate by testing each.

### Product Feedback Survey

Psychoactive effects of the ACP-containing compositions will be measured using a Product Feedback Survey. Volunteers will be asked to test the product by keeping the pen vertical with the metal end facing down, and inhaling from the mouth piece for 3-5 seconds. Volunteers will be asked to do this three times per product, and wait 30 minutes to complete a survey. Volunteers will be limited to one trial per day, in order to avoid the effects of one trial from bleeding into the next.

Volunteers will complete the survey described in Examples 1-3. Results for this survey will be presented as 1) average scores for each category, and 2) baseline subtracted scores to tease out the specific effects of each ACP formulation. Results will also be presented as a graph similar to that of FIG. 2.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### Further Embodiments of the Invention

Other subject matter contemplated by the present disclosure is set out in the following numbered embodiments:

1. An apparatus for providing atomized beta caryophyllene, comprising:

a beta caryophyllene reservoir configured to hold a liquid beta caryophyllene composition;

a beta caryophyllene atomizer in fluid communication with the beta caryophyllene reservoir;

an actuator coupled to the beta caryophyllene atomizer and configured such that, when actuated, the actuator initiates atomization of liquid beta caryophyllene composition at the beta caryophyllene atomizer;

a beta caryophyllene vapor chamber in fluid communication with the beta caryophyllene atomizer and configured to receive atomized beta caryophyllene composition; and

a mouthpiece in fluid communication with the beta caryophyllene vapor chamber and configured to provide atomized beta caryophyllene composition from the beta caryophyllene vapor chamber to a user.

1.1 The apparatus of embodiment 1, wherein the beta caryophyllene composition is substantially free from THC or CBD.
2. The apparatus of embodiment 1, wherein the beta caryophyllene composition consists essentially of beta caryophyllene.
2.1 The apparatus of embodiment 1, wherein the beta caryophyllene composition comprises less than 25% wt. beta caryophyllene.
2.2 The apparatus of embodiment 1, wherein the beta caryophyllene composition comprises less than 15% wt. beta caryophyllene.
2.3 The apparatus of embodiment 1, wherein the beta caryophyllene composition comprises less than 10% wt. beta caryophyllene.
3. The apparatus of embodiment 1, wherein the beta caryophyllene composition consists of beta caryophyllene.
4. The apparatus of embodiment 1, wherein the beta caryophyllene composition comprises at least 60° % beta caryophyllene.
5. The apparatus of embodiment 1, wherein the beta caryophyllene composition comprises at least 70% beta caryophyllene.
6. The apparatus of embodiment 1, wherein the beta caryophyllene composition comprises at least 80% beta caryophyllene.
7. The apparatus of embodiment 1, wherein the beta caryophyllene composition comprises at least 90/c beta caryophyllene.
8. The apparatus of embodiment 1, wherein the beta caryophyllene composition comprises at least 95% beta caryophyllene.
9. The apparatus of embodiment 1, wherein the beta caryophyllene composition comprises at least 99% beta caryophyllene.
10. The apparatus of any one of embodiments 4 to 9, wherein the beta caryophyllene composition further comprises isocaryophyllene and/or α-humulene.
11. The apparatus of any one of embodiments 2.1-2.3 and 4 to 9, wherein the beta caryophyllene composition consists essentially of beta caryophyllene, isocaryophyllene, and α-humulene.
12. The apparatus of any one of embodiments 2.1-2.3 and 4 to 9, wherein the beta caryophyllene composition consists essentially of beta caryophyllene and isocaryophyllene.
13. The apparatus of any one of embodiments 2.1-2.3 and 4 to 9, wherein the beta caryophyllene composition consists essentially of beta caryophyllene and α-humulene.
14. The apparatus of any one of embodiments 2.1-2.3 and 4 to 9, wherein the beta caryophyllene composition consists of beta caryophyllene, isocaryophyllene, and α-humulene.
15. The apparatus of any one of embodiments 2.1-2.3 and 4 to 9, wherein: (a) the beta caryophyllene composition consists of beta caryophyllene and isocaryophyllene; or (b) the beta caryophyllene composition consists of beta caryophyllene and α-humulene.
16. The apparatus of any one of embodiments 4 to 9, wherein the beta caryophyllene composition comprises (a) at least about 88% beta caryophyllene and (b) between about 8% and about 10% alpha caryophyllene, and is free or essentially free from both THC and CBD.
16.1 The apparatus of any one of embodiments 1 to 2.3, 4-13 and 16, wherein the beta caryophyllene composition comprises a carrier.
16.2 The apparatus of embodiment 16.1, wherein the carrier is selected from the group consisting of MCT, glycerine, vegetable glycerine and propylene glycol.
17. A method of treatment, comprising using the apparatus of any one of embodiments 1 to 16.2 to administer an atomized beta caryophyllene composition to a patient in need thereof.
18. The method of treatment of embodiment 17, wherein administering the atomized beta caryophyllene composition to the patient in need thereof comprises administering a dose of less than 10 mg of atomized beta caryophyllene composition to the patient.
19. The method of treatment of embodiment 17, wherein administering the atomized beta caryophyllene composition to the patient in need thereof comprises administering a dose of less than 9 mg of atomized beta caryophyllene composition to the patient.
20. The method of treatment of embodiment 17, wherein administering the atomized beta caryophyllene composition to the patient in need thereof comprises administering a dose of less than 8 mg of atomized beta caryophyllene composition to the patient.
21. The method of treatment of embodiment 17, wherein administering the atomized beta caryophyllene composition to the patient in need thereof comprises administering a dose of less than 7 mg of atomized beta caryophyllene composition to the patient.
22. The method of treatment of embodiment 17, wherein administering the atomized beta caryophyllene composition to the patient in need thereof comprises administering a dose of less than 6 mg of atomized beta caryophyllene composition to the patient.
23. The method of treatment of embodiment 17, wherein administering the atomized beta caryophyllene composition to the patient in need thereof comprises administering a dose of less than 5 mg of atomized beta caryophyllene composition to the patient.
24. The method of treatment of embodiment 17, wherein administering the atomized beta caryophyllene composition to the patient in need thereof comprises administering a dose of less than 4 mg of atomized beta caryophyllene composition to the patient.
25. The method of treatment of embodiment 17, wherein administering the atomized beta caryophyllene composition to the patient in need thereof comprises administering a dose of less than 3 mg of atomized beta caryophyllene composition to the patient.
26. The method of treatment of embodiment 17, wherein administering the atomized beta caryophyllene composition to the patient in need thereof comprises administering a dose of less than 2.5 mg of atomized beta caryophyllene composition to the patient.
27. The method of treatment of embodiment 17, wherein administering the atomized beta caryophyllene composition to the patient in need thereof comprises administering a dose of less than 2 mg of atomized beta caryophyllene composition to the patient.
28. The method of treatment of embodiment 17, wherein administering the atomized beta caryophyllene composition to the patient in need thereof comprises administering a dose of less than 1.5 mg of atomized beta caryophyllene composition to the patient.
29. The method of treatment of embodiment 17, wherein administering the atomized beta caryophyllene composition to the patient in need thereof comprises administering a dose of less than 1 mg of atomized beta caryophyllene composition to the patient.
30. The method of treatment of embodiment 17, wherein administering the atomized beta caryophyllene composition to the patient in need thereof comprises administering a dose of less than 0.5 mg of atomized beta caryophyllene composition to the patient.
31. The method of treatment of any one of embodiments 17-30, wherein the patient in need thereof suffers from one or more of the following: one or more attention disorders, one or more sleep disorders, depression, and/or pain
32. An apparatus for providing atomized alpha caryophyllene, comprising:

an alpha caryophyllene reservoir configured to hold a liquid alpha caryophyllene composition;

an alpha caryophyllene atomizer in fluid communication with the alpha caryophyllene reservoir;

an actuator coupled to the alpha caryophyllene atomizer and configured such that, when actuated, the actuator initiates atomization of liquid alpha caryophyllene composition at the alpha caryophyllene atomizer;

an alpha caryophyllene vapor chamber in fluid communication with the alpha caryophyllene atomizer and configured to receive atomized alpha caryophyllene composition; and

a mouthpiece in fluid communication with the alpha caryophyllene vapor chamber and configured to provide atomized alpha caryophyllene composition from the alpha caryophyllene vapor chamber to a user.

33. The apparatus of embodiment 32, wherein the alpha caryophyllene composition consists essentially of alpha caryophyllene.
34. The apparatus of embodiment 32, wherein the alpha caryophyllene composition consists of alpha caryophyllene.
35. The apparatus of embodiment 32, wherein the alpha caryophyllene composition comprises at least 60% alpha caryophyllene.
36. The apparatus of embodiment 32, wherein the alpha caryophyllene composition comprises at least 70% alpha caryophyllene.
37. The apparatus of embodiment 32, wherein the alpha caryophyllene composition comprises at least 80% alpha caryophyllene.
38. The apparatus of embodiment 32, wherein the alpha caryophyllene composition comprises at least 90% alpha caryophyllene.
39. The apparatus of embodiment 32, wherein the alpha caryophyllene composition comprises at least 95% alpha caryophyllene.
40. The apparatus of embodiment 32, wherein the alpha caryophyllene composition comprises at least 99% alpha caryophyllene.
40.1 The apparatus of embodiment 32, wherein the alpha caryophyllene composition comprises less than 25% wt. alpha caryophyllene.
40.2 The apparatus of embodiment 32, wherein the alpha caryophyllene composition comprises less than 10% wt. alpha caryophyllene.
40.3 The apparatus of embodiment 32, wherein the alpha caryophyllene composition comprises less than 5% wt. alpha caryophyllene.
40.4 The apparatus of embodiment 32, wherein the alpha caryophyllene composition comprises less than 3% wt. alpha caryophyllene.
40.5 The apparatus of embodiment 32, wherein the alpha caryophyllene composition comprises less than 2% wt. alpha caryophyllene.
40.6 The apparatus of embodiment 32, wherein the alpha caryophyllene composition comprises less than 1% wt. alpha caryophyllene.
40.7 The apparatus of embodiment 32, wherein the alpha caryophyllene composition comprises less than 0.75% wt. alpha caryophyllene.
41. The apparatus of any one of embodiments 35 to 40.7, wherein the alpha caryophyllene composition further comprises isocaryophyllene and/or beta caryophyllene.
42. The apparatus of any one of embodiments 35 to 40.7, wherein the alpha caryophyllene composition consists essentially of alpha caryophyllene, isocaryophyllene, and beta caryophyllene.
43. The apparatus of any one of embodiments 35 to 40.4, wherein the alpha caryophyllene composition consists essentially of alpha caryophyllene and isocaryophyllene.
44. The apparatus of any one of embodiments 35 to 40.7, wherein the alpha caryophyllene composition consists essentially of alpha caryophyllene and beta caryophyllene.
45. The apparatus of any one of embodiments 35 to 40.7, wherein the alpha caryophyllene composition consists of alpha caryophyllene, isocaryophyllene, and beta caryophyllene.
46. The apparatus of any one of embodiments 35 to 40, wherein the alpha caryophyllene composition consists of alpha caryophyllene and isocaryophyllene.
47. The apparatus of any one of embodiments 35 to 40.7, wherein the alpha caryophyllene composition consists of alpha caryophyllene and beta caryophyllene.
47.1 The apparatus of any one of embodiments 32, 33 and 35-44, wherein the alpha caryophyllene composition comprises a carrier.
47.2 The apparatus of embodiment 47.1, wherein the carrier is selected from the group consisting of MCT, glycerine, vegetable glycerine and propylene glycol.
48. A method of treatment, comprising using the apparatus of any one of embodiments 32 to 47 to administer a atomized alpha caryophyllene composition to a patient in need thereof.
49. The method of treatment of embodiment 48, wherein administering the atomized alpha caryophyllene composition to the patient in need thereof comprises administering a dose of less than 10 mg of atomized alpha caryophyllene composition to the patient.
50. The method of treatment of embodiment 48, wherein administering the atomized alpha caryophyllene composition to the patient in need thereof comprises administering a dose of less than 9 mg of atomized alpha caryophyllene composition to the patient.
51. The method of treatment of embodiment 48, wherein administering the atomized alpha caryophyllene composition to the patient in need thereof comprises administering a dose of less than 8 mg of atomized alpha caryophyllene composition to the patient.
52. The method of treatment of embodiment 48, wherein administering the atomized alpha caryophyllene composition to the patient in need thereof comprises administering a dose of less than 7 mg of atomized alpha caryophyllene composition to the patient.
53. The method of treatment of embodiment 48, wherein administering the atomized alpha caryophyllene composition to the patient in need thereof comprises administering a dose of less than 6 mg of atomized alpha caryophyllene composition to the patient.
54. The method of treatment of embodiment 48, wherein administering the atomized alpha caryophyllene composition to the patient in need thereof comprises administering a dose of less than 5 mg of atomized alpha caryophyllene composition to the patient.
55. The method of treatment of embodiment 48, wherein administering the atomized alpha caryophyllene composition to the patient in need thereof comprises administering a dose of less than 4 mg of atomized alpha caryophyllene composition to the patient.
56. The method of treatment of embodiment 48, wherein administering the atomized alpha caryophyllene composition to the patient in need thereof comprises administering a dose of less than 3 mg of atomized alpha caryophyllene composition to the patient.
57. The method of treatment of embodiment 48, wherein administering the atomized alpha caryophyllene composition to the patient in need thereof comprises administering a dose of less than 2.5 mg of atomized alpha caryophyllene composition to the patient.
58. The method of treatment of embodiment 48, wherein administering the atomized alpha caryophyllene composition to the patient in need thereof comprises administering a dose of less than 2 mg of atomized alpha caryophyllene composition to the patient.
59. The method of treatment of embodiment 48, wherein administering the atomized alpha caryophyllene composition to the patient in need thereof comprises administering a dose of less than 1.5 mg of atomized alpha caryophyllene composition to the patient.
60. The method of treatment of embodiment 48, wherein administering the atomized alpha caryophyllene composition to the patient in need thereof comprises administering a dose of less than 1 mg of atomized alpha caryophyllene composition to the patient.
61. The method of treatment of embodiment 48, wherein administering the atomized alpha caryophyllene composition to the patient in need thereof comprises administering a dose of less than 0.5 mg of atomized alpha caryophyllene composition to the patient.
62. The method of treatment of any one of embodiments 48-61, wherein the patient in need thereof suffers from one or more of the following: one or more attention disorders, one or more sleep disorders, depression, and/or pain
63. An vaporizable caryophyllene composition comprising beta caryophyllene and a carrier.
63.1 The vaporizable caryophyllene composition of embodiment 63, wherein the beta caryophyllene composition is substantially free from THC or CBD.
63.2 The vaporizable caryophyllene composition of embodiments 63 or 63.1 wherein the carrier is selected from the group consisting of MCT, glycerine, vegetable glycerine and propylene glycol
64. The vaporizable caryophyllene composition of any one of embodiments 63 or 63.1-63.2, wherein the caryophyllene composition consists essentially of beta caryophyllene.
64.1 The vaporizable caryophyllene composition of any one of embodiments 63 or 63.1-63.2, wherein the caryophyllene composition comprises less than 25% wt. beta caryophyllene.
64.2 The vaporizable caryophyllene composition of any one of embodiments 63 or 63.1-63.2, wherein the caryophyllene composition comprises less than 15% wt. beta caryophyllene.
64.2 The vaporizable caryophyllene composition of any one of embodiments 63 or 63.1-63.2, wherein the caryophyllene composition comprises less than 10% wt. beta caryophyllene.
65. The vaporizable caryophyllene composition of any one of embodiments 63 to 64.2, wherein the caryophyllene composition further comprises isocaryophyllene and/or α-humulene.
66. The vaporizable caryophyllene composition of any one of embodiments 63 to 64.2, wherein the caryophyllene composition consists essentially of beta caryophyllene, isocaryophyllene, and α-humulene.
67. The vaporizable caryophyllene composition of any one of embodiments 63 to 64.2, wherein the caryophyllene composition consists essentially of beta caryophyllene and isocaryophyllene.
68. The vaporizable caryophyllene composition of any one of embodiments 63 to 64.2, wherein the caryophyllene composition consists essentially of beta caryophyllene and α-humulene.
69. The vaporizable caryophyllene composition of any one of embodiments 63 to 64.2, wherein the caryophyllene composition consists of beta caryophyllene, isocaryophyllene, and α-humulene.
70. The vaporizable caryophyllene composition of any one of embodiments 63 to 64.2, wherein: (a) the caryophyllene composition consists of beta caryophyllene and isocaryophyllene; or (b) the beta caryophyllene composition consists of beta caryophyllene and α-humulene.
71. The vaporizable caryophyllene composition of any one of embodiments 63 to 64.2, wherein the caryophyllene composition comprises (a) at least about 88% beta caryophyllene and (b) between about 8% and about 10% alpha caryophyllene, and is free or essentially free from both THC and CBD.

1. An apparatus for providing atomized beta caryophyllene, comprising:
a beta caryophyllene reservoir configured to hold a liquid beta caryophyllene composition;
a beta caryophyllene atomizer in fluid communication with the beta caryophyllene reservoir;
an actuator coupled to the beta caryophyllene atomizer and configured such that, when actuated, the actuator initiates atomization of liquid beta caryophyllene composition at the beta caryophyllene atomizer;
a beta caryophyllene vapor chamber in fluid communication with the beta caryophyllene atomizer and configured to receive atomized beta caryophyllene composition; and
a mouthpiece in fluid communication with the beta caryophyllene vapor chamber and configured to provide atomized beta caryophyllene composition from the beta caryophyllene vapor chamber to a user.

2. The apparatus of claim 1, wherein the beta caryophyllene composition consists essentially of beta caryophyllene.

3. The apparatus of claim 1, wherein the beta caryophyllene composition consists of beta caryophyllene.

4. The apparatus of claim 1, wherein the beta caryophyllene composition comprises at least 60% beta caryophyllene.

5. The apparatus of claim 1, wherein the beta caryophyllene composition comprises at least 70% beta caryophyllene.

6. The apparatus of claim 1, wherein the beta caryophyllene composition comprises at least 80% beta caryophyllene.

7. The apparatus of claim 1, wherein the beta caryophyllene composition comprises at least 90% beta caryophyllene.

8. The apparatus of claim 1, wherein the beta caryophyllene composition comprises at least 95% beta caryophyllene.

9. The apparatus of claim 1, wherein the beta caryophyllene composition comprises at least 99% beta caryophyllene.

10. The apparatus of any one of claims 4 to 9, wherein the beta caryophyllene composition further comprises isocaryophyllene and/or α-humulene.

11. The apparatus of any one of claims 4 to 9, wherein the beta caryophyllene composition consists essentially of beta caryophyllene, isocaryophyllene, and α-humulene.

12. The apparatus of any one of claims 4 to 9, wherein the beta caryophyllene composition consists essentially of beta caryophyllene and isocaryophyllene.

13. The apparatus of any one of claims 4 to 9, wherein the beta caryophyllene composition consists essentially of beta caryophyllene and α-humulene.

14. The apparatus of any one of claims 4 to 9, wherein the beta caryophyllene composition consists of beta caryophyllene, isocaryophyllene, and α-humulene.

15. The apparatus of any one of claims 4 to 9, wherein: (a) the beta caryophyllene composition consists of beta caryophyllene and isocaryophyllene; or (b) the beta caryophyllene composition consists of beta caryophyllene and α-humulene.

16. The apparatus of any one of claims 4 to 9, wherein the beta caryophyllene composition comprises (a) at least about 88% beta caryophyllene and (b) between about 8% and about 10% alpha caryophyllene, and is free or essentially free from both THC and CBD.

17. A method of treatment, comprising using the apparatus of any one of claims 1 to 16 to administer an atomized beta caryophyllene composition to a patient in need thereof.

18. The method of treatment of claim 17, wherein administering the atomized beta caryophyllene composition to the patient in need thereof comprises administering a dose of less than 10 mg of atomized beta caryophyllene composition to the patient.

19. The method of treatment of claim 17, wherein administering the atomized beta caryophyllene composition to the patient in need thereof comprises administering a dose of less than 9 mg of atomized beta caryophyllene composition to the patient.

20. The method of treatment of claim 17, wherein administering the atomized beta caryophyllene composition to the patient in need thereof comprises administering a dose of less than 8 mg of atomized beta caryophyllene composition to the patient.

21. The method of treatment of claim 17, wherein administering the atomized beta caryophyllene composition to the patient in need thereof comprises administering a dose of less than 7 mg of atomized beta caryophyllene composition to the patient.

22. The method of treatment of claim 17, wherein administering the atomized beta caryophyllene composition to the patient in need thereof comprises administering a dose of less than 6 mg of atomized beta caryophyllene composition to the patient.

23. The method of treatment of claim 17, wherein administering the atomized beta caryophyllene composition to the patient in need thereof comprises administering a dose of less than 5 mg of atomized beta caryophyllene composition to the patient.

24. The method of treatment of claim 17, wherein administering the atomized beta caryophyllene composition to the patient in need thereof comprises administering a dose of less than 4 mg of atomized beta caryophyllene composition to the patient.

25. The method of treatment of claim 17, wherein administering the atomized beta caryophyllene composition to the patient in need thereof comprises administering a dose of less than 3 mg of atomized beta caryophyllene composition to the patient.

26. The method of treatment of claim 17, wherein administering the atomized beta caryophyllene composition to the patient in need thereof comprises administering a dose of less than 2.5 mg of atomized beta caryophyllene composition to the patient.

27. The method of treatment of claim 17, wherein administering the atomized beta caryophyllene composition to the patient in need thereof comprises administering a dose of less than 2 mg of atomized beta caryophyllene composition to the patient.

28. The method of treatment of claim 17, wherein administering the atomized beta caryophyllene composition to the patient in need thereof comprises administering a dose of less than 1.5 mg of atomized beta caryophyllene composition to the patient.

29. The method of treatment of claim 17, wherein administering the atomized beta caryophyllene composition to the patient in need thereof comprises administering a dose of less than 1 mg of atomized beta caryophyllene composition to the patient.

30. The method of treatment of claim 17, wherein administering the atomized beta caryophyllene composition to the patient in need thereof comprises administering a dose of less than 0.5 mg of atomized beta caryophyllene composition to the patient.

31. The method of treatment of any one of claims 17-30, wherein the patient in need thereof suffers from one or more of the following: one or more attention disorders, one or more sleep disorders, depression, and/or pain

32. An apparatus for providing atomized alpha caryophyllene, comprising:
an alpha caryophyllene reservoir configured to hold a liquid alpha caryophyllene composition;
an alpha caryophyllene atomizer in fluid communication with the alpha caryophyllene reservoir;
an actuator coupled to the alpha caryophyllene atomizer and configured such that, when actuated, the actuator initiates atomization of liquid alpha caryophyllene composition at the alpha caryophyllene atomizer;
an alpha caryophyllene vapor chamber in fluid communication with the alpha caryophyllene atomizer and configured to receive atomized alpha caryophyllene composition; and
a mouthpiece in fluid communication with the alpha caryophyllene vapor chamber and configured to provide atomized alpha caryophyllene composition from the alpha caryophyllene vapor chamber to a user.

33. The apparatus of claim 32, wherein the alpha caryophyllene composition consists essentially of alpha caryophyllene.

34. The apparatus of claim 32, wherein the alpha caryophyllene composition consists of alpha caryophyllene.

35. The apparatus of claim 32, wherein the alpha caryophyllene composition comprises at least 600/o alpha caryophyllene.

36. The apparatus of claim 32, wherein the alpha caryophyllene composition comprises at least 70% alpha caryophyllene.

37. The apparatus of claim 32, wherein the alpha caryophyllene composition comprises at least 80% alpha caryophyllene.

38. The apparatus of claim 32, wherein the alpha caryophyllene composition comprises at least 90% alpha caryophyllene.

39. The apparatus of claim 32, wherein the alpha caryophyllene composition comprises at least 95% alpha caryophyllene.

40. The apparatus of claim 32, wherein the alpha caryophyllene composition comprises at least 99% alpha caryophyllene.

41. The apparatus of any one of claims 35 to 40, wherein the alpha caryophyllene composition further comprises isocaryophyllene and/or beta caryophyllene.

42. The apparatus of any one of claims 35 to 40, wherein the alpha caryophyllene composition consists essentially of alpha caryophyllene, isocaryophyllene, and beta caryophyllene.

43. The apparatus of any one of claims 35 to 40, wherein the alpha caryophyllene composition consists essentially of alpha caryophyllene and isocaryophyllene.

44. The apparatus of any one of claims 35 to 40, wherein the alpha caryophyllene composition consists essentially of alpha caryophyllene and beta caryophyllene.

45. The apparatus of any one of claims 35 to 40, wherein the alpha caryophyllene composition consists of alpha caryophyllene, isocaryophyllene, and beta caryophyllene.

46. The apparatus of any one of claims 35 to 40, wherein the alpha caryophyllene composition consists of alpha caryophyllene and isocaryophyllene.

47. The apparatus of any one of claims 35 to 40, wherein the alpha caryophyllene composition consists of alpha caryophyllene and beta caryophyllene.

48. A method of treatment, comprising using the apparatus of any one of claims 32 to 47 to administer a atomized alpha caryophyllene composition to a patient in need thereof.

49. The method of treatment of claim 48, wherein administering the atomized alpha caryophyllene composition to the patient in need thereof comprises administering a dose of less than 10 mg of atomized alpha caryophyllene composition to the patient.

50. The method of treatment of claim 48, wherein administering the atomized alpha caryophyllene composition to the patient in need thereof comprises administering a dose of less than 9 mg of atomized alpha caryophyllene composition to the patient.

51. The method of treatment of claim 48, wherein administering the atomized alpha caryophyllene composition to the patient in need thereof comprises administering a dose of less than 8 mg of atomized alpha caryophyllene composition to the patient.

52. The method of treatment of claim 48, wherein administering the atomized alpha caryophyllene composition to the patient in need thereof comprises administering a dose of less than 7 mg of atomized alpha caryophyllene composition to the patient.

53. The method of treatment of claim 48, wherein administering the atomized alpha caryophyllene composition to the patient in need thereof comprises administering a dose of less than 6 mg of atomized alpha caryophyllene composition to the patient.

54. The method of treatment of claim 48, wherein administering the atomized alpha caryophyllene composition to the patient in need thereof comprises administering a dose of less than 5 mg of atomized alpha caryophyllene composition to the patient.

55. The method of treatment of claim 48, wherein administering the atomized alpha caryophyllene composition to the patient in need thereof comprises administering a dose of less than 4 mg of atomized alpha caryophyllene composition to the patient.

56. The method of treatment of claim 48, wherein administering the atomized alpha caryophyllene composition to the patient in need thereof comprises administering a dose of less than 3 mg of atomized alpha caryophyllene composition to the patient.

57. The method of treatment of claim 48, wherein administering the atomized alpha caryophyllene composition to the patient in need thereof comprises administering a dose of less than 2.5 mg of atomized alpha caryophyllene composition to the patient.

58. The method of treatment of claim 48, wherein administering the atomized alpha caryophyllene composition to the patient in need thereof comprises administering a dose of less than 2 mg of atomized alpha caryophyllene composition to the patient.

59. The method of treatment of claim 48, wherein administering the atomized alpha caryophyllene composition to the patient in need thereof comprises administering a dose of less than 1.5 mg of atomized alpha caryophyllene composition to the patient.

60. The method of treatment of claim 48, wherein administering the atomized alpha caryophyllene composition to the patient in need thereof comprises administering a dose of less than 1 mg of atomized alpha caryophyllene composition to the patient.

61. The method of treatment of claim 48, wherein administering the atomized alpha caryophyllene composition to the patient in need thereof comprises administering a dose of less than 0.5 mg of atomized alpha caryophyllene composition to the patient.

62. The method of treatment of any one of claims 48-61, wherein the patient in need thereof suffers from one or more of the following: one or more attention disorders, one or more sleep disorders, depression, and/or pain
